(19) United States
(12) Patent Application Publication
(10) Pub. No.: US 2019/0300868 A1
(43) Pub. Date: Oct. 3, 2019

(54) COMPOSITIONS AND METHODS FOR CRISPR-BASED SCREENING

(71) Applicant: The Regents of the University of California, Oakland, CA (US)

(72) Inventors: Luke Gilbert, San Francisco, CA (US); Maximilian A. Horlbeck, San Francisco, CA (US); Marco Jost, San Francisco, CA (US); Jonathan Weissman, San Francisco, CA (US)

(21) Appl. No.: 16/469,098

(22) PCT Filed: Dec. 15, 2017
(86) PCT No.: PCT/US2017/066842

§ 371 Date: Jun. 12, 2019

Related U.S. Application Data
(60) Provisional application No. 62/434,778, filed on Dec. 15, 2016.

(51) Int. Cl. C12N 9/22; C12N 9/12; C12Q 1/6869; C12N 15/11; C12N 15/86

(52) U.S. Cl. CPC C12N 9/22; C12N 9/1247; C12Q 1/6869; C12N 2320/12; C12N 15/86; C12N 2310/20; C12N 15/11

(57) ABSTRACT

Provided herein are compositions and methods for CRISPR-based screening.

### PRIOR RELATED APPLICATION

This application is a U.S. National Phase under 35 USC § 371 of PCT Application No. PCT/US2017/066842, filed Dec. 15, 2017, which claims priority to U.S. Provisional Application No. 62/434,778, filed Dec. 15, 2016, the disclosures of which are hereby incorporated by reference in their entireties for all purposes.

### STATEMENT AS TO RIGHTS TO INVENTIONS MADE UNDER FEDERALLY SPONSORED RESEARCH AND DEVELOPMENT

This invention was made with government support under Grant No. K99 CA204602 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED ON A COMPACT DISK

This application includes a Sequence Listing as a text file named “Sequence Listing for 081906-1143815-224910US.txt” created Jun. 11, 2019, and containing 11,456 bytes. The material contained in this text file is incorporated by reference in its entirety for all purposes.

### BACKGROUND OF THE INVENTION

Clustered, regularly interspaced short palindromic repeat (CRISPR) screening has become a dominant technology for identification of genes required for cellular processes. The potential for conducting comprehensive, genome-scale CRISPR screening in diploid eukaryotic systems is enormous, but in practice the utility has been hampered by a number of technical challenges. Genomic screens often identify large numbers of ‘hit’ genes, but there is no systematic method for understanding how these genes may function together. Therefore, compositions and methods for reliably identifying genes from CRISPR genomic screens that are required for cellular processes are of interest.

### BRIEF SUMMARY OF THE INVENTION

In some embodiments, the present invention provides a nucleic acid construct comprising multiple expression cassettes wherein each expression cassette comprises: a) a polynucleotide sequence comprising an RNA polymerase III promoter operably linked to a nucleic acid encoding a small guide RNA (sgRNA) comprising a DNA targeting sequence and a constant region that interacts with a site-directed nuclease; and b) a pair of unique barcode sequences that flank the polynucleotide sequence comprising the RNA polymerase III promoter operably linked to the nucleic acid encoding a small guide RNA (sgRNA), wherein the RNA polymerase III promoter in each cassette of the nucleic acid construct has a different sequence.

In some examples, the constant region of the sgRNA in each cassette of the nucleic acid construct has a different sequence. In some examples, the constant region of the sgRNA in each cassette of the nucleic acid construct has an identical sequence. In some examples, the nucleic acid construct has two expression cassettes. In some examples the nucleic acid construct has three expression cassettes. In some examples, the RNA polymerase III promoters are from different mammalian species. In some examples, the sgRNA interacts with an enzymatically active site-directed nuclease. In some examples, the enzymatically active site-directed nuclease is a Cas9 polypeptide. In some examples, the sgRNA interacts with a deactivated site-directed nuclease. In some examples, the deactivated site-directed nuclease is a deactivated Cas9 (dCas9) polypeptide.

In some examples, a vector comprises the nucleic acid construct. In some examples, the vector is a lentiviral vector.

In some embodiments, the present invention provides a method for sequencing a first and a second sgRNA that target a first and a second DNA target in a genome of a cell, the method comprising: a) infecting a plurality of mammalian cells with a plurality of vectors to form a plurality of vector-infected cells, wherein each vector comprises: i) a first polynucleotide sequence comprising a first RNA polymerase III promoter operably linked to a nucleic acid encoding a first sgRNA comprising a sequence that targets a first DNA target in the genome and a first constant region that interacts with a site directed nuclease; and a pair of unique barcode sequences that flank the polynucleotide sequence comprising the RNA polymerase III promoter operably linked to the nucleic acid encoding the first sgRNA; and ii) a second polynucleotide sequence comprising a second RNA polymerase III promoter operably linked to a nucleic acid encoding a second sgRNA comprising a sequence that targets a second DNA target in the genome and a second constant region that interacts with a site directed nuclease; and a pair of unique barcode sequences that flank the polynucleotide sequence comprising the RNA polymerase III promoter operably linked to the nucleic acid encoding the second sgRNA; and b) expressing a site-directed nuclease in the mammalian cells; c) separating a selected pool of cells expressing a detectable phenotype from the plurality of infected cells; d) amplifying DNA comprising the nucleic acid encoding the first sgRNA and the nucleic acid encoding the second sgRNA in each cell with a pair of primers; wherein optionally, at least one of the primers includes a sample barcode sequence, and wherein the amplified DNA contains two adjacent barcodes flanked by the first and second sgRNAs; e) sequencing the nucleic acid encoding the first sgRNA and the nucleic acid encoding the second sgRNA in each cell; f) optionally sequencing the sample barcode; g) sequencing both adjacent barcode sequences to obtain a barcode sequence combination for each cell; h) comparing the barcode sequence combination obtained from each cell with the combination of the unique barcode sequence of the first sgRNA and the unique barcode sequence of the second sgRNA in the cell; and i) identifying the first and the second sgRNA that target a first and a second DNA target in cells comprising a combination of barcode sequences corresponding to the combination of the unique barcode sequence of the first sgRNA and the unique barcode sequence of the second sgRNA in the cell.

In some examples, the first sgRNA and the second sgRNA are sequences on the same strand of amplified DNA and the adjacent barcode sequences are sequenced on the opposite strand of the amplified DNA. In some examples, the sample barcode sequence is optionally sequenced from the same strand of the amplified DNA or the opposite strand of the amplified DNA.

In some examples, the vector is a lentiviral vector. In some examples, the method further comprises infecting the mammalian cells with a vector comprising a polynucleotide sequence encoding the site-directed nuclease prior to or subsequent to infecting the cells with the plurality of vectors.

In some examples, the first RNA polymerase III promoter and the second RNA polymerase III promoter have different sequences. In some examples, the first constant region and the second constant region have different sequences. In some examples, the first constant region and the second constant region have identical sequences.

In some examples, the site-directed nuclease is an enzymatically active site-directed nuclease. In some examples, enzymatically active site-directed nuclease is a Cas9 polypeptide. In some examples, the site-directed nuclease is a deactivated site-directed nuclease. In some examples, the deactivated site-directed nuclease is a dCas9 polypeptide.

In some examples, the dCas9 polypeptide is linked to a transcriptional activator. In some examples, the dCas9 polypeptide is linked to a transcriptional activator and the method further comprises constructing a gain-of-function genetic interaction map. In some examples, the dCas9 polypeptide is linked to a transcriptional inhibitor. In some examples, the dCas9 polypeptide is linked to a transcriptional inhibitor and the method further comprises constructing a loss-of-function genetic interaction map.

### BRIEF DESCRIPTION OF THE DRAWINGS

The present application includes the following figures. The figures are intended to illustrate certain embodiments and/or features of the compositions and methods, and to supplement any description(s) of the compositions and methods. The figure does not limit the scope of the compositions and methods, unless the written description expressly indicates that such is the case.

FIG. 1 depicts a strategy for systematic genetic modifier screens using single cell expression profiling. (A) Schematic of the Perturb-seq platform. CBC, cell barcode (index unique to each bead). UMI, unique molecular identifier (index unique to each bead oligo). GBC, guide barcode (index unique to each sgRNA). (B) Schematic of the Perturb-seq vector and guide-mapping amplicon. (C) Performance of GBC capture. Top 3 possible GBCs for each CBC. CBC identity was assigned to sgRNA identity when a single GBC dominated and any lower abundance GBCs were rejected. CBC was identified as a “multiplet” when a second or third GBC also had good coverage. Compare with (D,E). (D) Distribution of captured UMIs from dominant guide-mapping amplicons. (E) Performance of perturbation (sgRNA) identification. Data also represented in FIG. 8B. (F) Kernel density estimates of normalized flow cytometry counts representing GFP expression and knockdown achieved from the indicated sgRNA expression constructs. See also FIG. 8.

FIG. 2 depicts a strategy for multiplexed delivery of CRISPR guide RNAs in a single expression vector. (A) Schematic of the final three-guide Perturb-seq vector. “PS” denotes protospacer. (B) Kernel density estimates of normalized flow cytometry counts representing GFP expression and knockdown achieved from the indicated sgRNA expression constructs. (C) Top: Schematic of sgRNA constant region with indicated changes. Orange, cr2 changes. Purple, cr3 changes. Bottom: Relative RFP from an E. coli CRISPRi reporter strain expressing an sgRNA with the indicated constant region variant and an mRFP-targeting protospacer. Data represent mean fluorescence of replicates normalized to negative control sgRNA±standard deviations (n=3). (D) Kernel density estimates of normalized flow cytometry counts representing GFP expression and knockdown achieved from the indicated sgRNA expression constructs. For details on one-guide vectors see FIG. 9F and Methods. (E) Kernel density estimates of normalized flow cytometry counts representing GFP expression and knockdown achieved from the indicated sgRNA expression constructs. Data for the Perturb-seq vector is the same as in panel (B). (F) Average percent mRNA remaining after simultaneous repression of ERN1 (IRE1α), EIF2AK3 (PERK), and ATF6 using a final three-guide Perturb-seq vector determined via Perturb-seq. See also FIG. 9.

FIG. 3 shows epistatic analysis of the three transcriptional arms of the unfolded proteins response using perturb-seq. (A) Schematics of the unfolded protein response (UPR) and Perturb-seq UPR epistasis experiment. (B) Unbiased identification and decoupling of single-cell behaviors via low rank independent component analysis (LRICA) in UPR epistasis experiment. Gene expression in cells (dots) is reduced to components identifying major trends in the population. Plots show t-sne projections of components that vary across genetic perturbations and chemical treatments (bottom left) or cell cycle position (bottom right). Tg, thapsigargin. DMSO-treated control cells (+DMSO) contain non-targeting control sgRNAs (throughout FIG. 3). (C) Plots (t-sne) of perturbation subpopulations (indicated GBC/treatment pairs: +DMSO and Tg-treated cells with or without PERK) from UPR epistasis experiment. LRICA identified a component (IC) that is bimodal within each of these subpopulations and marks G1 cells. (D) Cell cycle composition of perturbation subpopulations from panel (C). (E) Perturbation subpopulations from panel (C) were further divided into G1 and non-G1 cells based on IC value. Heatmap displays normalized expression of the 50 genes that most influenced IC, exposing both synergistic and antagonistic interactions. (F) Genetic interactions among the three branches of the UPR. Top: Heatmap displays average expression profiles of 104 genes that strongly varied within the UPR epistasis experiment for each perturbation (i.e. indicated GBC/treatment pairs). Genes were clustered by their expression pattern within the entire population (i.e. all cells in all conditions). These patterns determine the branch specificity of each gene. Bottom: Unbiased decomposition of the total response into three components obtained via ICA. See also FIG. 10.

FIG. 4 shows genome-scale CRISPRi screening for genetic stresses that perturb the IRE1 branch of the unfolded protein response. (A) Schematic of UPRE and constitutive EF1α reporter cassettes (see Methods in Example I). (B) K562 reporter (cBA011) cells were transduced with an XBP1-targeting sgRNA and treated with 2 μg/mL tunicamycin or DMSO after 4 days. Approximately 12 hours later, these cells were evaluated by flow cytometry. Data is representative of two independent experiments. (C) Schematic of CRISPRi screens. (D) Volcano plot of gene reporter phenotypes and p-values from CRISPRi-v2 screen. Data generated from negative control sgRNAs and screen hits are shown. (E) Gene reporter phenotypes from CRISPRi-v2 screen (as in D) by functional category. Screen hits are shown. (F) Comparison of UPRE and EF1α signals from K562 reporter (cBA011) cells transduced with 257 sgRNAs targeting 152 hit genes from the CRISPRi-v2 screen and 3 distinct negative controls. Data represent logy averages of background-adjusted fluorescence medians (normalized to untransduced cells) collected from four separate experiments (n=2-7 replicates). See also FIG. 11.

FIG. 5 shows the results of a large-scale perturb-seq experiment interrogating ER homeostatis. (A) Clustering of genes from UPR Perturb-seq experiment. Heatmap displays correlations between hierarchically clustered average expression profiles from all cells bearing sgRNAs targeting the same gene (identified by GBCs). Functional annotations are indicated. (B) Change in cell cycle composition induced by indicated genetic perturbations (identified by GBC) relative to control (NegCtrl-2) cells. (C) Average percent target mRNA remaining from each subpopulation (identified by GBC). Genes targeted by multiple sgRNAs have multiple, possibly overlapping dots. Error bars are 95% CI estimated by bootstrapping. (D) Individually evaluated UPRE signal phenotypes (data for hit genes also represented in FIG. 4F) and scores measuring activation of the three UPR branches for each genetic perturbation. Final panel represents the log₁₀ number of genes differentially expressed relative to control cells measured by the Kolmogorov-Smirnov test at P<0.01. See also FIG. 12.

FIG. 6 shows that single-cell information reveals a bifurcated UPR within a population and allows unbiased discovery of UPR-controlled genes. (A) Single-cell projections (t-sne) of sgRNA identity, cell cycle position, and UMI count per cell in HSPA5-perturbed and control cells (containing the NegCtrl-3 guide). We note that the HSPA5-targeting sgRNAs indicated differ by only 1-nt. (B) LRICA analysis of HSPA5-perturbed cells identifies two subpopulation-defining independent components. Right panel: subpopulations defined by thresholding IC1. (C) Branch activation scores in HSPA5-perturbed cells. (D) Normalized expression of UPR genes in HSPA5-perturbed cells. Each row is a cell, ordered by increasing IC1, and each column is a gene in the same order as FIG. 3F. (E) Mean expression of HSPA5 across subpopulations. Error bars are 95% CI. (F) Cell cycle composition of HSPA5-perturbed cells. (G) Strategy for using correlated expression to identify functionally related genes. (H) Unbiased identification of induced gene expression programs. Top: Normalized expression of 200 genes with significantly altered expression in UPR Perturb-seq experiment clustered based on co-expression. Bottom: Normalized expression in UPR epistasis experiment, to assess UPR dependence. Full version in FIG. 13A. (I) UPR-responsive genes with altered expression in the UPR Perturb-seq experiment clustered by co-expression in the UPR epistasis experiment, the UPR Perturb-seq experiment, and control cells. Cophenetic correlation coefficients between dendrograms along with a visual guide (represented by colors) to the movement of major groups included. Full version in FIG. 13B. (J) Strategy for enriching cells perturbed for a trait of interest. (K) Within cells enriched for a set of bait cholesterol biosynthesis genes, a group of genes clustered with the bait genes and had more correlated expression than in control cells. Reactome annotations and SREBP binding data for the group included (right panel). See also FIG. 13.

FIG. 7 shows that translocon loss-of function preferentially activates IRE1 UPR signaling. (A) Single-cell analysis of SEC61B-perturbed cells in UPR Perturb-seq experiment. Control cells contain the NegCtrl-3 guide. (B) Analysis of SEC61A1-perturbed cells (as in A). (C) XBP1 mRNA splicing from cells transduced with the indicated sgRNAs and treated +/−thapsigargin (0.5 μM Tg for 1.5 hours). (D) XBP1 mRNA splicing (top) and SSR2 and CHOP mRNA expression (bottom) from cells transduced with the indicated sgRNAs. Graphical data represent means relative to ACTS mRNA and normalized to cells transfected with NegCtrl-1 sgRNA (dotted lines)±standard error of technical replicates (n=3). XBP1u, unsliced. XBP1s, spliced. (E) Relative CHOP mRNA in cells described in (C). Data represent means relative to ACTB mRNA and normalized to cells transfected with NegCtrl-3 sgRNA±standard error of technical replicates (n=3). (F) Model of translocon feedback signaling through IRE1α. See also FIG. 14.

FIG. 8 provides an overview of experiments related to FIG. 1. (A) Pilot experiment schematic. sgRNAs targeting 7 transcription factors and 1 negative control were individually transduced into K562 cells with dCas9-KRAB (cBA010), which were then pooled prior to Perturb-seq. (B) Statistics of pilot experiment. Because transductions were performed individually, “multiplets” in this case may arise either from multiple encapsulation events during emulsion generation or PCR artifacts. (C) UPR epistasis experiment schematic. Using our final three-guide Perturb-seq vector to simultaneously deliver 3 sgRNAs, we individually transduced K562 cells expressing dCas9-KRAB (cBA010) with constructs that targeted all three UPR sensor genes in every combination (singly with controls, doubly with a control, or triply). Transduced cells were then pooled and selected. After 2 days of combined growth, the cells were treated with DMSO for 6 hours, 4 μg/mL tunicamycin (Tm) for 6 hours, or 100 nM thapsigargin (Tg) for 4 hours and were profiled by Perturb-seq (24 conditions in total). (D) Statistics of UPR epistasis experiment. Multiplet definition is as in (B). (E) UPR Perturb-seq experiment schematic. Lentiviruses were individually prepared from Perturb-seq vectors encoding 93 sgRNAs, including 2 controls. These were then pooled and used to transduce K562 cells with dCas9-KRAB (cBA010) prior to selection, outgrowth, and Perturb-seq. (F) Statistics of UPR Perturb-seq experiment. “Multiplets” in this case include the categories in (B), as well as multiple infections during the pooled transduction.

FIG. 9 shows the design and characterization of a three-guide vector. (A) Characterization of initial three-guide vector by GFP knockdown. GFP+ K562 cells with dCas9-KRAB were transduced with either a one-guide perturb-seq vector expressing a GFP-targeting guide RNA, initial three-guide vectors expressing a GFP-targeting guide RNA from the promoter indicated in parentheses and negative control guide RNAs from the other two promoters, or a one-guide vector expressing a negative control guide RNA. GFP levels were measured by flow cytometry 10 day after transduction. Plotted are kernel density estimates of normalized flow cytometry counts for infected (BFP+) cells. Traces for the single perturb-seq construct and the negative control are the same as in FIG. 2E. (B) Characterization of h7SK promoter in the context of the perturb-seq vector. Experiment was conducted as described in (A). Traces for the one-guide perturb-seq construct and the negative control are the same as in FIG. 2E. (C) Characterization of GFP+ K562 cells with increased dCas9-KRAB levels. BFP levels report on expression level of the dCas9-BFP-KRAB construct. Increase in dCas9-KRAB is measured by change in BFP fluorescence relative to WT K562 cells. Plotted are kernel density estimates of normalized flow cytometry counts. (D) Crystal structure of Cas9 bound to guide RNA and target DNA (PDB ID code 4008 (Nishimasu et al. 2014)), highlighting location of constant region mutations. Cas9 is shown in gray, target ssDNA in yellow, and the guide RNA in orange (targeting region) and cyan (constant region). Constant region bases that were mutated are highlighted in red. (E) Characterization of RNA polymerase III promoters from different mammalian species by GFP knockdown. GFP+ K562 cells with dCas9-KRAB were transduced with vectors expressing a GFP-targeting guide RNA from the different promoters, in the context of the perturb-seq vector. GFP levels were measured by flow cytometry either 9 days (experiment 1) or 8 day after transduction (experiment 2). Percentage knockdown was calculated after subtracting GFP autofluorescence from wild type K562 cells as measurements relative to GFP+ K562 cells transduced with a negative control vector. Abbreviations: bU6, bovine U6; sU6, sheep U6; buU6, buffalo U6; pU6, pig U6. (F) Cloning strategy for final three-guide vector. In step 1, protospacers are ligated into individual backbones. In step 2, three-guide RNA expression cassettes are amplified by PCR and inserted into the perturb-seq backbone in a single reaction by four-piece Gibson assembly to obtain the final barcoded three-guide vector.

FIG. 10 shows a perturb-seq analytical pipeline related to FIG. 3. (A) Schematic of the analytical pipeline. Each step is explained in the Methods, and each single-cell figure has a dedicated section in the Methods describing its construction. (B) Example analysis of thapsigargin-treated cells, related to FIG. 3B. The left panels show t-sne projections of the whole population derived using all differentially expressed genes, as described in the Methods. The middle panels show the 16 independent components found by low rank ICA overlaid on the t-sne plot. The right panels shows how four of the components (IC1-IC4) vary in average value across the different perturbation subpopulations, and how four distinct components (IC5-IC8) vary in average value across the cell cycle. When present, specific labels of the components are inferred from these patterns. Further details are in the Methods.

FIG. 11 shows the results of CRISPRi screens, related to FIG. 4, used to select UPR-modulating sgRNAs for perturb-seq. (A) K562 reporter cells (cBA011) were treated with indicated concentrations of tunicamycin (Tm) in 0.16% DMSO or DMSO alone. Cells were evaluated by flow cytometry or collected for analysis of XBP1 mRNA splicing at indicated times. Graphical data represent average of background-adjusted median fluorescence normalized to DMSO control cells±standard deviation (n=3). (B) Comparison of gene reporter phenotypes from the CRISPRi-v1 and CRISPRi-v2 screens. Genes chosen for analysis on the Perturb-seq platform (83) are indicated in red (C) Reporter phenotypes of sgRNAs from replicates of CRISPRi-v2 screen. Gray indicates negative control sgRNAs. (D) Comparison of gene reporter phenotypes from the CRISPRi-v2 screen with gene growth phenotypes from a previously reported genome-scale CRISPRi-v2 screen (Horlbeck et al., 2016). Select hits are indicated in red. (E) Top eleven annotated functional clusters from DAVID enrichment analysis. Representative names were chosen for each cluster.

FIG. 12 shows perturb-seq screen performance in experiments related to FIG. 5. (A) Similarity of phenotypes between sgRNAs targeting the same gene. Average expression profiles were created for each sgRNA-containing subpopulation, and hierarchically clustered. Guides targeting a common gene are indicated by color. (B) Shift in sgRNA target expression upon depletion. The distribution of expression of each targeted gene is compared between control cells (containing the NegCtrl-2 guide) and each sgRNA-containing subpopulation. sgRNAs are ordered by target expression. (C) Homogeneity of knockdown. We computationally separated each sgRNA-containing subpopulation into top- and bottom-third most perturbed cells based on the deviation of their RNA-seq profiles from the distribution of expression seen in control cells (Methods). The plot shows the average difference in percentage knockdown between these two subpopulations for each sgRNA (gray dot), along with a kernel density estimate of the distribution (black). (D) Expression of UPR genes in UPR Perturb-seq experiment. The plot shows the average normalized expression within each gene-perturbed subpopulation of all of the genes identified as UPR-responsive in FIG. 3F. The thapsigargin data from that figure is repeated to the right for comparison. (E) Alternate scoring system for branch activation. An alternative method of scoring branch activation was developed using independent component analysis. For comparison with FIG. 5D. See Methods for details. (F) Performance of random forest scoring system for branch activation. To cross-validate branch activation scores, three alternative scoring systems were prototyped in the UPR epistasis experiment of FIG. 3, where branch activation can be inferred from perturbation identity (i.e. each GBC for a given treatment): (1) group score, based on expression of defined gene lists, (2) ICA score, seen in FIG. 12E, (3) random forest score, seen in FIG. 5D. Cells were defined as “active” for a given branch if they were treated with tunicamycin or thapsigargin and that branch of the UPR was not depleted. The plots show the distributions of scores in active and inactive cells for each of the three branches and each of the three scoring systems, which ideally should separate these two classes. See Methods for details.

FIG. 13 shows the results of functionally clustering genes using single-cell correlation information related to FIG. 6. (A) Full-size version of FIG. 6H. 200 genes were identified that were induced broadly in the UPR Perturb-seq experiment and then clustered based on co-expression (Methods). The heatmap shows the average normalized expression of the given gene (column) within a particular perturbation subpopulation (row, shown in the same order as FIG. 5A). Analogous data from the UPR epistasis experiment is shown in the bottom panel for comparison, to allow UPR-dependent genes to be identified. Group labels were added based on presumed biological function. (B) Full-size version of FIG. 61. We identified UPR-regulated genes (from FIG. 3F) that showed the same pattern of induction or repression in response to the perturbations in the UPR Perturb-seq experiment (removing any whose expression was not substantially altered). These genes were then clustered based on co-expression from the UPR epistasis experiment, the UPR Perturb-seq experiment, or control cells (from the UPR Perturb-seq experiment bearing the NegCtrl-2 guide). The heatmap shown is the average normalized expression of those genes in the indicated conditions from the UPR epistasis experiment, to assess branch specificity. The colors on the dendrograms identify how these major groups shift when the different cell populations are used for clustering.

FIG. 14 shows depletion of individual translocon components SEC61A1, SEC61B, or SEC61G upregulate expression of complex partner genes by have distinct growth phenotypes. (A) ATF6 and PERK branch activation scores for SEC61B- and SEC61A1-perturbed subpopulations and controls. For comparison with FIG. 7A. (B) K562 cells (cBA011) transduced and sorted for expression of BFP, which marks successful transduction of constructs carrying the indicated sgRNAs, were collected 6 days post transduction for analysis of SEC61A1, SEC61B, SEC61G, and ALG2 expression. Data represent means relative to ACTS mRNA and normalized to cells transfected with NegCtrl-3±standard error of technical replicates (n=3). Samples from the cells with NegCtrl-1, SEC61A1-2, and SEC61B-2 sgRNAs were also evaluated in FIG. 7D. In this experiment sgRNAs were expressed from the original sgRNA expression vector (Addgene, Cat #60955). (C) Phenotypes for individual sgRNAs targeting SEC61A1, SEC61B, and SEC61G from growth screens, reported and conducted as described elsewhere (Horlbeck et al., 2016). Data for 10 library negative control sgRNAs were randomly chosen for inclusion. Guides used separately elsewhere are numbered.

FIG. 15 depicts a sequencing strategy for detecting and discarding intermolecular recombination events. (A) Exemplary schematic of sequencing of sgRNAs and corresponding unique barcodes in amplified DNA from a cell where intermolecular recombination has not occurred. (B) Exemplary schematic of sequencing of sgRNAs and corresponding unique barcodes in amplified DNA from a cell where intermolecular recombination has occurred.

FIG. 16 shows a strategy for generating GI maps in human cells. (A) Map of the dual sgRNA-expressing lentiviral vector and primer sites for the triple sequencing strategy. Read 1 corresponds to Illumina sample read 1, read 2 corresponds to Illumina index read 1, Illumina index read 2 is used for sample multiplexing (not depicted), and read 3 corresponds to Illumina sample read 2. (B) Schematic of a dual-sgRNA screening protocol in human cells.

FIG. 17 shows sgRNA single phenotypes in the A or B position analyzed by three different sequencing alignment strategies. sgRNA single phenotypes were calculated as the average of the targeting sgRNA in the indicated position paired with non-targeting control sgRNAs in the other position. Error bars represent standard deviation. sgRNA read counts were calculated by aligning barcodes (Top), sgRNAs (Middle), or only matching sgRNAs and barcodes (Bottom).

FIG. 18 shows sgRNA single phenotypes in the A or B position compared to the same sgRNA in both positions. sgRNA single phenotypes were calculated as in FIG. 17 and plotted against the phenotypes for the corresponding sgRNA in both A and B positions.

FIG. 19 shows sgRNA single phenotypes in the A or B position analyzed by three different sequencing alignment strategies from the Jurkat screen. sgRNA single phenotypes were calculated as the average of the targeting sgRNA in the indicated position paired with non-targeting control sgRNAs in the other position. Error bars represent standard deviation. sgRNA read counts were calculated by aligning barcodes (Top), sgRNAs (Middle), or only matching sgRNAs and barcodes (Bottom).

FIG. 20 shows sgRNA single phenotypes in the A or B position compared to the same sgRNA in both positions from the Jurkat screen. sgRNA single phenotypes were calculated as in FIG. 17 and plotted against the phenotypes for the corresponding sgRNA in both A and B positions.

FIG. 21 is a schematic of a genetic interaction map analysis pipeline.

FIG. 22 shows the results of analysis of sgRNA epistasis from single and pair phenotypes. (A) sgRNA single phenotypes versus pair phenotypes for three representative query sgRNAs. The slope, curvature, and variance of the relationship depended on the single phenotype of the query sgRNA. Thus, a quadratic fit of the relationship forced through the intercept at the query sgRNA single phenotype was used to determine the expected pair phenotype. Epistasis was then calculated as the difference of the measured pair phenotype and the expected phenotype, z-standardized to the standard deviation of the negative control-query sgRNA pairs. (B) Effect of quadratic fit and z-standardization on the mean and standard deviation of GIs for each query sgRNA. Red indicates negative control query sgRNAs. Using a linear fit, mean GI was related to the single phenotype. This relationship was corrected using a quadratic fit. Standard deviation of GI score increased with stronger single phenotypes, likely as pairs with those sgRNAs were overall less well represented at endpoint. Z-standardization corrected this relationship, but notably all targeting query sgRNAs had larger GI standard deviations than negative controls. (C) Correlation between sgRNA pair GIs calculated from individual replicates. Contours are as in FIG. 27A.

FIG. 23 shows the correspondence between replicate sgRNA pair phenotypes. Replicate experiments were conducted from independent infections of the sgRNA pair lentiviral library in K562s. Pair phenotypes were calculated from the log 2 enrichment of pair counts in the endpoint sample compared to T0 (top) and normalized to the number of cell doublings in the replicate to obtain γ (middle). Replicates were then averaged together, and pairs with sgRNAs in the AB and BA position were compared (bottom). Contours are as in FIG. 27A.

FIG. 24 shows correspondence between replicate sgRNA pair phenotypes for Jurkat. Replicate experiments were conducted from independent infections of the sgRNA pair lentiviral library in Jurkat cells. Pair phenotypes were calculated from the log 2 enrichment of pair counts in the endpoint sample compared to T0 and normalized to the number of cell doublings in the replicate to obtain γ (left). Replicates were then averaged together, and pairs with sgRNAs in the AB and BA position were compared (right). Contours are as in FIG. 27A.

FIG. 25 shows the results of a large-scale quantitative GI mapping platform in human cells. (A) Schematic of overall GI mapping approach. (B) Histogram of gene growth phenotypes (γ) from a CRISPRi v1 growth screen (Gilbert et al., 2014). A subset of genes were selected for inclusion in the GI map, primarily based on exhibiting a moderate growth phenotype. (C) Cellular processes represented in GI map, with number of genes in parentheses (D) Subcellular localizations of proteins encoded by genes in the GI map (see Methods in Example II). (E) Approach for quantifying epistasis between sgRNAs, based on the relationship between single sgRNA phenotypes and the corresponding pair phenotypes with a given “query” sgRNA. (F) Example of sgRNA epistasis with query sgRNA sgANAPC13-1. Negative control sgRNAs are circled in red, and red line corresponds to quadratic fit of all sgRNA single and pair phenotypes (see also FIG. 22).

FIG. 26 shows individual validation of sgRNA epistasis. (A) Schematic of individual validation strategy. (B) Single versus pair phenotypes for query sgRNA sgUBA2-2. (C) Individual validation of indicated buffering sgRNA pairs with sgUBA2-2, expressed as log 2 enrichment relative to day 4.

FIG. 27 shows results from a large-scale CRISPRi-based GI map. (A) sgRNA GI correlations from two independent replicates. Contours correspond to 99th, 95th, 90th, 75th, 50th, and 25th percentiles of data density. Pearson correlation (R) is of all sgRNA pair correlations. Due to the size of the dataset, Pearson p-values here and throughout the manuscript are <10⁻³⁰⁰ unless otherwise stated. (B) Histogram of sgRNA GI correlations calculated from replicated-averaged sgRNA pair phenotypes. (C) Full gene-level GI map. Dendrogram indicates average linkage hierarchical clustering based on Pearson correlations between genes. Bars denote clusters containing poorly characterized genes.

FIG. 28 shows sgRNA GI correlations for pairs targeting genes in the indicated complex.

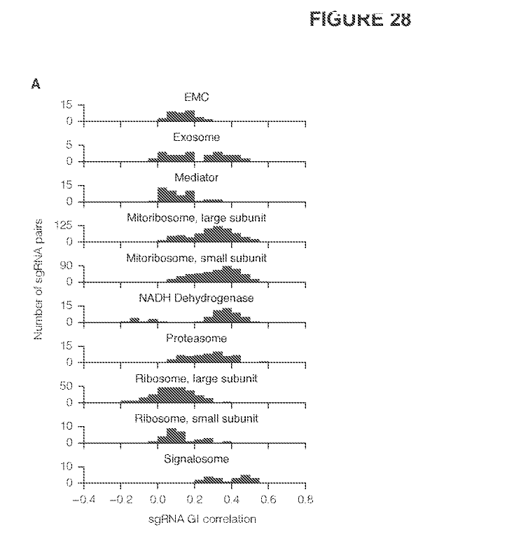

FIG. 29 provides determinants of intra-complex GI correlation. (A) Relationship between sgRNA median read count and intra-complex GI correlation. Intra-complex GI correlations were higher for better represented sgRNAs. In addition, using a more stringent filter (e.g. 35 reads used for GI map) eliminated poorly correlating sgRNAs and increased the correlation of remaining sgRNAs due to reduced noise. (B) Histogram of gene-level GI correlations for all gene pairs and for intra-complex gene pairs. (C) Gene-level GI correlations for pairs targeting genes in the indicated complex. (D) Intra-complex gene-level GI correlations for genes targeted by 1, 2, or 3 sgRNAs in the sgRNA-level map. (E) Relationship between sgRNA-level and gene-level intra-complex GI correlation. For genes with more than one targeting sgRNA, lines connect the minimum and maximum sgRNA-level intra-complex correlation.

FIGS. 30A-C show excerpts from the full gene-level GI map.

FIG. 31 shows that GI correlations identify members of protein complexes and functionally related pathways. (A) Histogram of all correlations between gene GI profiles or between non-targeting control and gene GI profiles (black). (B) Cumulative distribution of GI correlations for all genes (as in A), for gene pairs within mitochondria or early trafficking, or for pairs with one gene in each compartment. (C) Fraction of gene pairs with a given GI correlation annotated by the STRING experimentally validated interaction set. GI correlations were binned to the next-lowest tenth. (D) Gene networks of all highly correlated genes. Edges represent correlations greater than 0.6. GI correlations that correspond both to STRING-annotated interactions and to MitoCarta gene pairs were labeled according to their STRING interaction confidence. Edge lengths were determined by force-directed layout. Asterisk indicate gene pairs that have closely neighboring TSSs. (E) Selected interactions with TMEM261 and NADH Dehydrogenase complex members. (F) Selected interactions with ASNA1 and CAMLG.

FIG. 32 shows the results of an analysis of GI correlations. (A) GI correlations for GI maps generated from individual replicates. Contours are as in FIG. 27A. (B) Cumulative distribution of GIs for all gene pairs or pairs annotated in the STRING experimentally validated interaction set at the indicated level of interaction confidence. (C) UCSC genome browser track for the SLC39A9 and ERH TSS overlap locus. (D) Distance between gene pair TSSs and GI correlation. Blue line represents median of all gene pairs within 10-fold of the point. Dark blue swath represents 25th to 75th percentile interval, and light blue represents 5th to 95th. (E) Histogram of distance to any coding TSS for all genes in the GI map.

FIG. 33 shows that glycolytic and oxidative metabolism exhibit anti-correlated GI profiles. (A) GI scores for genes paired with ATP5A1 and PGK1 (B) GI correlation with ATP5A1 for genes involved in carbon metabolism.

FIG. 34 provides an analysis of GI scores (A) GI scores for GI maps generated from individual replicates. Contours are as in FIG. 27A. (B) Histogram of all GI scores, as in FIG. 36A, as well as GI scores for genes paired with negative control genes.

FIG. 35 shows the structure of genetic interactions in the draft GI map. (A) Histogram of all GI scores. (B) Relationship between GI correlation and score. GI correlations were binned to the next-lowest tenth. (Left) Boxplot of scores within each bin. (Middle) Percent strong buffering interactions within each bin. (Right) Percent strong synergistic interactions within each bin. (C) Enrichment of correlations and interactions for gene pairs between the indicated cellular compartments. Values indicate percent of all gene pairs between compartments that are strongly correlated or interacting.

FIG. 36 shows the results of an analysis of an sgRNA-level GI map in Jurkat cells. (A) sgRNA GI correlations from two independent replicates. Contours are as in FIG. 2A. Pearson correlation (R) is of all sgRNA pair correlations. (B) Histogram of sgRNA GI correlations calculated from replicated-averaged sgRNA pair phenotypes. (C) sgRNA GI correlations for pairs targeting genes in the indicated complex. (D) Correlation between sgRNA pair GIs calculated from individual replicates. Contours are as in FIG. 27A.

FIG. 37 shows the results of an analysis of gene-level GI map in Jurkat cells. (A) GI scores for GI maps generated from individual replicates. Contours are as in FIG. 2A. (B) Histogram of all GI scores, as well as GI scores for genes paired with negative control genes. (C) GI correlations for GI maps generated from individual replicates. Contours are as in FIG. 2A. (D) Histogram of all correlations between gene GI profiles or between non-targeting control and gene GI profiles (black). (E) Gene-level GI correlations for pairs targeting genes in the indicated complex.

FIG. 38 shows the results of sampling for expansion to to a complete GI map of the human cell. (A) Coherence of a sub-sampled rectangular GI map. Columns were randomly sub-sampled from the GI map, GI correlations were calculated from the rows, and Spearman rank correlation with GI correlations from the full GI map was calculated. 100 random sub-samplings were performed for each number of remaining columns. (B) CRISPRi knockdown of the indicated genes. Genes were chosen from five tested in (Du et al., 2017). The top 3 predicted sgRNAs in the hCRISPRi-v2.1 library were cloned into lentiviral vectors and infected into K562 cells. Cells were puromycin selected and harvested, and mRNA expression was measured by qPCR and expressed relative to the NT sgRNA. Bars are average of 3 biological replicates, and error bars indicate standard deviation.

### DEFINITIONS

As used in this specification and the appended claims, the singular forms “a,” “an,” and “the” include plural reference unless the context clearly dictates otherwise.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

A “reporter gene” encodes proteins that are readily detectable due to their biochemical characteristics, such as enzymatic activity or chemifluorescent features. These reporter proteins can be used as selectable markers. One specific example of such a reporter is green fluorescent protein. Fluorescence generated from this protein can be detected with various commercially-available fluorescent detection systems. Other reporters can be detected by staining. The reporter can also be an enzyme that generates a detectable signal when contacted with an appropriate substrate. The reporter can be an enzyme that catalyzes the formation of a detectable product. Suitable enzymes include, but are not limited to, proteases, nucleases, lipases, phosphatases and hydrolases. The reporter can encode an enzyme whose substrates are substantially impermeable to eukaryotic plasma membranes, thus making it possible to tightly control signal formation. Specific examples of suitable reporter genes that encode enzymes include, but are not limited to, CAT (chloramphenicol acetyl transferase; Alton and Vapnek (1979) Nature 282: 864-869); luciferase (lux); β-galactosidase; LacZ; β.-glucuronidase; and alkaline phosphatase (Toh, et al. (1980) Eur. J. Biochem. 182: 231-238; and Hall et al. (1983) J. Mol. Appl. Gen. 2: 101), each of which are incorporated by reference herein in its entirety. Other suitable reporters include those that encode for a particular epitope that can be detected with a labeled antibody that specifically recognizes the epitope.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease in complex with guide and activating RNA to recognize and cleave foreign nucleic acid. Methods and compositions for controlling inhibition and/or activation of transcription of target genes, populations of target genes (e.g., controlling a transcriptome or portion thereof) are described, e.g., in Cell. 2014 Oct. 23; 159(3):647-61, the contents of which are incorporated by reference in the entirety for all purposes.

As used herein, “activity” in the context of CRISPR/Cas activity, Cas9 activity, sgRNA activity, sgRNA:nuclease activity and the like refers to the ability to bind to a target genetic element and/or modulate transcription at or near the target genetic element. Such activity can be measured in a variety of ways as known in the art. For example, expression, activity, or level of a reporter gene, or expression or activity of a gene encoded by the genetic element can be measured.

### DETAILED DESCRIPTION OF THE INVENTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Provided herein are compositions and methods for reducing intramolecular and intermolecular recombination events that corrupt genetic interaction mapping from CRISPR-based screens. By pairing sgRNAs with modified mammalian RNA polymerase III promoters, multiple sgRNAs can be expressed on a single construct, while eliminating recombination events. Barcode sequences are assigned to each sgRNA to identify any constructs that have undergone recombination after introduction of the construct into cells, for example, in a CRISPR screen. Methods for sequencing the sgRNAs and barcodes associated with each sgRNA are used to eliminate cells that have undergone a recombination event and identify cells that have not undergone a recombination event. By eliminating cells that have undergone a recombination event and only analyzing those cells that have not undergone a recombination event, nonspecific interactions and background noise can be eliminated from genetic interaction studies.

### Compositions

Compositions for targeting and modulating expression of nucleic acids in a cell are provided. Described herein are nucleic acid constructs comprising multiple expression cassettes wherein each expression cassette comprises: a) a polynucleotide sequence comprising an RNA polymerase III promoter operably linked to a nucleic acid encoding a small guide RNA (sgRNA) comprising a DNA targeting sequence and a constant region that interacts with a site-directed nuclease; and b) a pair of unique barcode sequences that flank the polynucleotide sequence comprising the RNA polymerase III promoter operably linked to the nucleic acid encoding a small guide RNA (sgRNA), wherein the RNA polymerase III promoter in each cassette of the nucleic acid construct has a different sequence.

In some examples, the constant region of the sgRNA in each cassette of the nucleic acid construct has a different sequence. In some examples, the constant region of the sgRNA in each cassette of the nucleic acid construct has the same or identical sequence. In other words, in some examples, all of the expression cassettes in the nucleic acid construct comprise an sgRNA with the same or identical constant region. In some examples, the nucleic acid construct comprises two, three, four, five, six, seven, eight, nine or more expression cassettes. In some examples, the nucleic acid construct comprises two, three, four, five, six, seven, eight, nine or more expression cassettes, wherein the constant region of the sgRNA in each cassette is the same or identical. In other examples, the nucleic acid construct comprises two, three, four, five, six, seven, eight, nine or more expression cassettes, wherein two or more of the constant regions of the sgRNAs in the nucleic acid construct have different sequences. In some examples, one or more of the expression cassettes further comprises a reporter gene or a nucleic acid encoding a reporter protein. Methods of making nucleic acid constructs comprising multiple expression cassettes are set forth in the Examples. See also, Gibson et al. Nature Methods 6, 343:343-345 (2009), for methods of enzymatically assembling multiple nucleic acid sequences.

In some examples, the RNA polymerase III promoter sequences are from different mammalian species. For example, the RNA polymerase III promoter sequences can be different RNA polymerase III promoter sequences from a human, cow, sheep, buffalo, pig or mouse, to name a few. In some examples, the RNA polymerase III promoter sequence is a U6 or an H1 sequence. In some examples, one or more of the RNA polymerase III sequences is a modified RNA polymerase III sequence. For example, one or more RNA polymerase III sequences having at least 80%, 85%, 90%, 95%, or 99% identity to a wild-type RNA polymerase III promoter sequence from any mammalian species can be used in the constructs provided herein. Examples of modified RNA polymerase III promoters are provided in Table 1.

TABLE 1
Modified RNA polymerase III promoters

| Species | |
|---|---|
| (abbreviation) | Sequence |
| human (hU6) | TATGGCGCGCCCCAAGGTCGGGCAGGAAGAGGGCCTATTTCCCATGATTCCTTC |
| | ATATTTGCATATACGATACAAGGCTGTTAGAGAGATAATTGGAATTAATTTGAC |
| | TGTAAACACAAAGATATTAGTACAAAATACGTGACGTAGAAAGTAATAATTTCT |
| | TGGGTAGTTTGCAGTTTTAAAATTATGTTTTAAAATGGACTATCATATGCTTACC |
| | GTAACTTGAAAGTATTTCGATTTCTTGGCTTTATATATCTTGTGGAAAGCCAGAA |
| | ACATGG (SEQ ID NO: 1) |
| mouse (mU6) | GATCCGACGCGCCATCTCTAGGCCCGCGCCGGCCCCCTCGCACGGACTTGTGGG |
| | AGAAGCTCGGCTACTCCCCTGCCCCGGTTAATTTGCATATAATATTTCCTAGTAA |
| | CTATAGAGGCTTAATGTGCGATAAAAGACAGATAATCTGTTCTTTTTAATACTA |
| | GCTACATTTTACATGATAGGCTTGGATTTCTATAACTTCGTATAGCATACATTAT |
| | ACGAAGTTATAAACAGCACAAAAGGAAACTCACCCTAACTGTAAAGTAATTGT |
| | GTGTTTTGAGACTATAAGTATCCCTTGGAGAACCACCTTGTTGG (SEQ ID |
| | NO: 2) |
| cow 2 (bU6-2) | CGTGACCGAGCTTGTCTGCCCGCGCAGTCCACTAGACAGACGCGCAGAGGTCGG |
| | GCGGGCCTAGGGCCAATTTCCCATGGTTCCCTTCATTTGCATATATGATGTAATA |
| | AGGTTATGGAGACTATTAAACTTAGCCCTAATCAAACTATATGATGATAATGTG |
| | TGTGGTACAAAAGGTCATAACTTATTATATACTTTGAAACTTAAAAAAGGGTTA |
| | CAGTTTAGTCACCATAACTGTAAAATTTTTTCTATTCTTAGCTTTATATAGTTCTT |
| | GAGAGGCCATGTTTATGG (SEQ ID NO: 3) |
| cow 3 (bU6-3) | AGCGCGCAGCTCGCTGCTCCCAGCATGCTCCACGGGCCGAGCACTCCCGCAGGA |
| | GGACCCGGGGCCCTGGGGCCCTCGACGAGACCCCCCCTTACCCAAGATGCTCCG |
| | GGCGCTCATTTGCATGTCCCACCAAGAGGTGAACTAGACTTGGTTTAGTTGTCG |
| | AGCACCAAACGTGCGAGCTTTGTGAGTGACTGCACCAAATCAAATTTCCCAAAA |
| | TGTTCCGCGTTCGCTGGGGAGAGGCGCAGCTTGGTCACCATAACTATAAAGGAG |
| | GAAAGATATCTGTACCTTATATATTCTGTGGGGGGTCCATGATTATGG (SEQ ID |
| | NO: 4) |
| buffalo (buU6) | CCACGTGACCGAGCTTGTCTGCCCGCGCAGTCCACTAGACGCACAGAGGTCGGG |
| | GCGGGCCTAGGACCAATTTCCCATGGTTCCCTTCATTTGCATATATGATGTAATA |
| | AGGTTATGAAGACTATTAAACTTAATAATAATCAAACAATATGATGATAGTGTG |
| | TGTGGTACAAAAGATCATAACTTATTATATACTTTGAAACTTAAAGAAGGGTTA |
| | CAGCTTAGTCACCATAACTGTAAAATTTTTTCTGTTCTTAGCTTTATATAGTTCTT |
| | GAGAAGCCATGTTTATGG (SEQ ID NO: 5) |
| pig (pU6) | TAGGGGAACGAGCACCACGTGACCGAGCGTGACAGTAAGGCGCGCGGAGGTCG |
| | GGCGGGCAGAGGGCCAATTTCCCATGGTCCCTTCATTTGCATATAATATGTAGG |
| | AAGGTTAAAGAGACAATTCTACTTAACTCTCCCAAAATCACAAAGATATGATGA |
| | CAGTATGTGTGACATAGAAGGTCATAAATCCTCGAATACTTTTAAATTGAAAAA |
| | AATTACTCTTCTATAGTCACCATAATCAAAAAAGTTTTTATGTTCTTGGCTTTAT |
| | ATACCCTCTGAGAAGCCACAGCCGTGG (SEQ ID NO: 6) |
| sheep 1 (sU6-1) | TCCCGGCATGCTCCGCGGGGCTCAGGGCCCCAAAGGCTCCATCGTCCGACTTGG |
| | AGTCGGTGACAGGGCCCTTACCCAAGATGCTACGGGCACTCATTTGCATGTCCC |
| | AGCAAGAGGAGAACTCGACAAGTTTCGCGATCTAGCAGTCAGAATACCAAACA |
| | TGCGAGCCTTGTGCGTGGATCTGTGAGATCAAATTTCCCTAAAAGTTTCAGAGT |
| | CATCGAAAAGATGCACGGTTTGGTCACCGTAACTGTAAAGAGATAAGACCTTGT |
| | GGGTTTTATATAAGCGGTGGACGCCAGCTGAATGG (SEQ ID NO: 7) |
| sheep 2 (sU6-2) | ACCACGTGACCGAGCTTGTCTGCCCTCGCAGACTACTAGGCGCGCAGAGGTCGG |
| | GCGGGGGCAGGGCCAATTTCCCATGGTTCCCTTCATTTGCATATATGATGTAAT |
| | AAGGTTATGGAGACTATTAAACTTAATCATAATCCAACAATATGATGACAGTAT |
| | GTGTGGTACAAAAGGGCAGAACTTATTATGTACTTTGAGTCTTAAAGAAGGATT |
| | ACAGTCTAGTCACCATAAGTGTAAAATTTTTTCTATTCATAGCTTTATATAGTTC |
| | CTGAGAGGCCACGTTTGTGG (SEQ ID NO: 8 |

Those of skill in the art readily understand how to determine the identity of two polypeptides or nucleic acids. For example, the identity can be calculated after aligning the two sequences so that the identity is at its highest level. Another way of calculating identity can be performed by published algorithms. For example, optimal alignment of sequences for comparison can be conducted using the algorithm of Needleman and Wunsch, J. Mol. Biol. 48: 443 (1970).

As used throughout, a sgRNA is a single guide RNA sequence that interacts with a site-directed nuclease and specifically binds to or hybridizes to a target nucleic acid within the genome of a cell, such that the sgRNA and the site-directed nuclease co-localize to the target nucleic acid in the genome of the cell. Each sgRNA includes a DNA targeting sequence or protospacer sequence of about 10 to 50 nucleotides in length that specifically binds to or hybridizes to a target DNA sequence in the genome. For example, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the sgRNA comprises a crRNA sequence and a transactivating crRNA (tracrRNA) sequence. In some embodiments, the sgRNA does not comprise a tracrRNA sequence.

Generally, the DNA targeting sequence is designed to complement (e.g., perfectly complement) or substantially complement (e.g., having 1-4 mismatches) to the target DNA sequence. In some cases, the DNA targeting sequence can incorporate wobble or degenerate bases to bind multiple genetic elements. In some cases, the 19 nucleotides at the 3′ or 5′ end of the binding region are perfectly complementary to the target genetic element or elements. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is preferably between about 40% and about 60% (e.g., 40%, 45%, 50%, 55%, 60%). In some cases, the binding region, can be selected to begin with a sequence that facilitates efficient transcription of the sgRNA. For example, the binding region can begin at the 5′ end with a G nucleotide. In some cases, the binding region can contain modified nucleotides such as, without limitation, methylated or phosphorylated nucleotides.

As used herein, the term “complementary” or “complementarity” refers to base pairing between nucleotides or nucleic acids, for example, and not to be limiting, base pairing between a sgRNA and a target nucleic acid. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence.

In some examples, the sgRNAs are targeted to specific regions at or near a gene. For example, an sgRNA can be targeted to a region at or near the 0-750 bp region 5′ (upstream) of the transcription start site of a gene. In some cases, the 0-750 bp targeting of the region can provide or provide increased, transcriptional activation by an sgRNA:deactivated site-directed nuclease complex. For example, the sgRNA can form a complex with a dCas9 polypeptide linked to a transcriptional activator to provide, or provide increased transcriptional activation of a gene by the complex. As another example, an sgRNA can be targeted to a region at or near the 0-1000 bp region 3′ (downstream) of the transcription start site of a gene. In some cases, the 0-1000 bp targeting of the region to provide, or provide increased, transcriptional repression by an sgRNA: deactivated site-directed complex. For example, the sgRNA can form a complex with a dCas9 polypeptide linked to a transcriptional inhibitor to provide, or provide increased transcriptional repression of a gene by the complex.

In some examples, the sgRNAs are targeted to a region at or near the transcription start site (TSS) based on an automated or manually annotated database. For example, transcripts annotated by Ensembl/GENCODE or the APPRIS pipeline (Rodriguez et al., Nucleic Acids Res. 2013 January; 41(Database issue):D110-7 can be used to identify the TSS and target genetic elements 0-750 bp upstream (e.g., for targeting one or more transcriptional activator domains) or 0-1000 bp downstream (e.g., for targeting one or more transcriptional repressor domains) of the TSS.

In some examples, the sgRNAs are targeted to a genomic region that is predicted to be relatively free of nucleosomes. The locations and occupancies of nucleosomes can be assayed through use of enzymatic digestion with micrococcal nuclease (MNase). MNase is an endo-exo nuclease that preferentially digests naked DNA and the DNA in linkers between nucleosomes, thus enriching for nucleosome-associated DNA. To determine nucleosome organization genome-wide, DNA remaining from MNase digestion is sequenced using high-throughput sequencing technologies (MNase-seq). Thus, regions having a high MNase-seq signal are predicted to be relatively occupied by nucleosomes and regions having a low MNase-seq signal are predicted to be relatively unoccupied by nucleosomes. Thus, in some examples, the sgRNAs are targeted to a genomic region that has a low MNase-Seq signal.

In some cases, the sgRNAs are targeted to a region predicted to be highly transcriptionally active. For example, the sgRNAs can be targeted to a region predicted to have a relatively high occupancy for RNA polymerase II (PolII). Such regions can be identified by PolII chromatin immunoprecipitation sequencing (ChIP-seq), which includes affinity purifying regions of DNA bound to PolII using an anti-PolII antibody and identifying the purified regions by sequencing. Therefore, regions having a high PolII Chip-seq signal are predicted to be highly transcriptionally active. Thus, in some cases, sgRNAs are targeted to regions having a high PolII ChIP-seq signal as disclosed in the ENCODE-published PolII ChIP-seq database (Landt, et al., Genome Research, 2012 September; 22(9):1813-31).

As another example, the sgRNAs can be targeted to a region predicted to be highly transcriptionally active as identified by run-on sequencing or global run-on sequencing (GRO-seq). GRO-seq involves incubating cells or nuclei with a labeled nucleotide and an agent that inhibits binding of new RNA polymerase to transcription start sites (e.g., sarkosyl). Thus, only genes with an engaged RNA polymerase produce labeled transcripts. After a sufficient period of time to allow global transcription to proceed, labeled RNA is extracted and corresponding transcribed genes are identified by sequencing. Therefore, regions having a high GRO-seq signal are predicted to be highly transcriptionally active. Thus, in some cases, sgRNAs are targeted to regions having a high GRO-seq signal as disclosed in a published GRO-seq data (e.g., Core et al., Science. 2008 Dec. 19; 322(5909):1845-8; and Hah et al., Genome Res. 2013 August; 23(8): 1210-23).

Each sgRNA also includes a cr/tracr RNA constant region that interacts with or binds to the site-directed nuclease. In the nucleic acid constructs provided herein, the constant region of an sgRNA can be from about 75 to 250 nucleotides in length. In some examples, the constant region is a modified constant region comprising one, two, three, four, five, six, seven, eight, nine, ten or more nucleotide substitutions in the stem, the stem loop, a hairpin, a region in between hairpins, and/or the nexus of a constant region. Any modified constant region that has at least 80%, 85%, 90%, or 95% activity, as compared to the activity of the natural or wild-type sgRNA constant region from which the modified constant region is derived, can be used in the constructs described herein. In some cases, the constant regions differ by one, two, three, four, five, six, seven, eight, nine, ten or more nucleotides. In particular, modifications should not be made at nucleotides that interact directly with a site-directed nuclease, for example, a Cas9 polypeptide, or at nucleotides that are important for the secondary structure of the constant region. Multiple constant regions can be designed to minimize interaction between the constant regions in the same nucleic acid construct. For example, and not to be limiting, constant regions that do not share more than about 15-20 nucleotides of consecutive sequence homology can be designed.

Non-limiting examples of constant regions that can be used in the constructs set forth herein are provided in Table 2. These variants were derived from the constant region described in Gilbert & Horlbeck (2014). In some examples, the nucleic acid sequences of constant regions cr1 (original constant region in Table 2), cr2 and cr3 are paired with different RNA polymerase III sequences provided herein. In some examples, the constant regions for the sgRNAs in the nucleic acid construct are the same. In some examples, the constant regions for the sgRNAs in the nucleic acid construct are different. In some examples, by pairing a different, constant region for each sgRNA sequence with a different RNA polymerase III promoter in each cassette, intramolecular recombination between sgRNA sequences can be prevented upon transduction of the construct into cells.

TABLE 2
Constant region variants.

| Variant | Description | Sequence |
|---|---|---|
| original | as described in Gilbert et | GTTTAAGAGCTAAGCTGGAAACAGCATAGCAAGTTTAAATAAGGCTAGTCCGTTATCAACT |
| | al. 2014 | TGAAAAAGTGGCACCGAGTCGGTGCTTTTTTT (SEQ ID NO: 9) |
| 1 | changed base pair in stem | GTCTAAGAGCTAAGCTGGAAACAGCATAGCAAGTTTAGATAAGGCTAGTCCGTTATCAACT |
| | | TGAAAAAGTGGCACCGAGTCGGTGCTTTTTTT (SEQ ID NO: 10) |
| 2 | changed base pair in stem | GTTTCAGAGCTAAGCTGGAAACAGCATAGCAAGTTGAAATAAGGCTAGTCCGTTATCAACT |
| | | TGAAAAAGTGGCACCGAGTCGGTGCTTTTTTT (SEQ ID NO: 11) |
| 3 | changed base pair in stem | GTTTGAGAGCTAAGCTGGAAACAGCATAGCAAGTTCAAATAAGGCTAGTCCGTTATCAACT |
| | | TGAAAAAGTGGCACCGAGTCGGTGCTTTTTTT (SEQ ID NO: 12) |
| 4 | scrambled stem loop | GTTTAAGAGCTAAGCACAAGAGTGCATAGCAAGTTTAAATAAGGCTAGTCCGTTATCAACT |
| | | TGAAAAAGTGGCACCGAGTCGGTGCTTTTTTT (SEQ ID NO: 13) |
| 5 | scrambled stem loop | GTTTAAGAGCTAAGCAGAAAGCTGCATAGCAAGTTTAAATAAGGCTAGTCCGTTATCAACT |
| | | TGAAAAAGTGGCACCGAGTCGGTGCTTTTTTT (SEQ ID NO: 14) |
| 6 | scrambled stem loop | GTTTAAGAGCTAAGCGCGAAAGCGCATAGCAAGTTTAAATAAGGCTAGTCCGTTATCAACT |
| | | TGAAAAAGTGGCACCGAGTCGGTGCTTTTTTT (SEQ ID NO: 15) |
| 7 | changed base pair in nexus | GTTTAAGAGCTAAGsCTGGAAACAGCATAGCAAGTTTAAATAAGACTAGTTCGTTATCAAC |
| | | TTGAAAAAGTGGCACCGAGTCGGTGCTTTTTTT (SEQ ID NO: 16) |
| 8 | changed bases at end of | GTTTAAGAGCTAAGCTGGAAACAGCATAGCAAGTTTAAATAAGGCTAGTCCGAATTCAACT |
| | nexus | TGAAAAAGTGGCACCGAGTCGGTGCTTTTTTT (SEQ ID NO: 17) |
| 9 | changed bases at end of | GTTTAAGAGCTAAGCTGGAAACAGCATAGCAAGTTTAAATAAGGCTAGTCCGTACACAACT |
| | nexus | TGAAAAAGTGGCACCGAGTCGGTGCTTTTTTT (SEQ ID NO: 18) |
| 10 | changed bases at end of | GTTTAAGAGCTAAGCTGGAAACAGCATAGCAAGTTTAAATAAGGCTAGTCCGTTTACAACT |
| | nexus | TGAAAAAGTGGCACCGAGTCGGTGCTTTTTTT (SEQ ID NO: 19) |
| 11 | changed base in bulge of | GTTTAAGAGCTAAGCTGGAAACAGCATAGCAAGTTTAAATAAGGCTAGTCCGTTATCAACT |
| | first hairpin | TGAGAAAGTGGCACCGAGTCGGTGCTTTTTTT (SEQ ID NO: 20) |
| 12 | inserted base between | GTTTAAGAGCTAAGCTGGAAACAGCATAGCAAGTTTAAATAAGGCTAGTCCGTTATCAACT |
| | hairpins | TGAAAAAGTGCGCACCGAGTCGGTGCTTTTTTT (SEQ ID NO: 21) |
| 13 | inserted base between | GTTTAAGAGCTAAGCTGGAAACAGCATAGCAAGTTTAAATAAGGCTAGTCCGTTATCAACT |
| | hairpins | TGAAAAAGTGAGCACCGAGTCGGTGCTTTTTTT (SEQ ID NO: 22) |
| 14 | inserted base pair in | GTTTAAGAGCTAAGCTGGAAACAGCATAGCAAGTTTAAATAAGGCTAGTCCGTTATCAACT |
| | second hairpin | TGAAAAAGTGGCACCCGAGTCGGGTGCTTTTTTT (SEQ ID NO: 23) |
| 15 | inserted base pair into | GTTTAAGAGCTAAGCTGGAAACAGCATAGCAAGTTTAAATAAGGCTAGTCCGTTATCAACT |
| | second hairpin | TGAAAAAGTGGCAGCCGAGTCGGCTGCTTTTTTT (SEQ ID NO: 24) |
| cr2 | combination of 3, 5, 9, 15 | GTTTGAGAGCTAAGCAGAAAGCTGCATAGCAAGTTCAAATAAGGCTAGTCCGTACACAACT |
| | | TGAAAAAGTGGCAGCCGAGTCGGCTGCTTTTTTT (SEQ ID NO: 25) |
| cr3 | combination of 2, 4, 10, 14 | GTTTCAGAGCTAAGCACAAGAGTGCATAGCAAGTTGAAATAAGGCTAGTCCGTTTACAACT |
| | | TGAAAAAGTGGCACCCGAGTCGGGTGCTTTTTTT (SEQ ID NO: 26) |

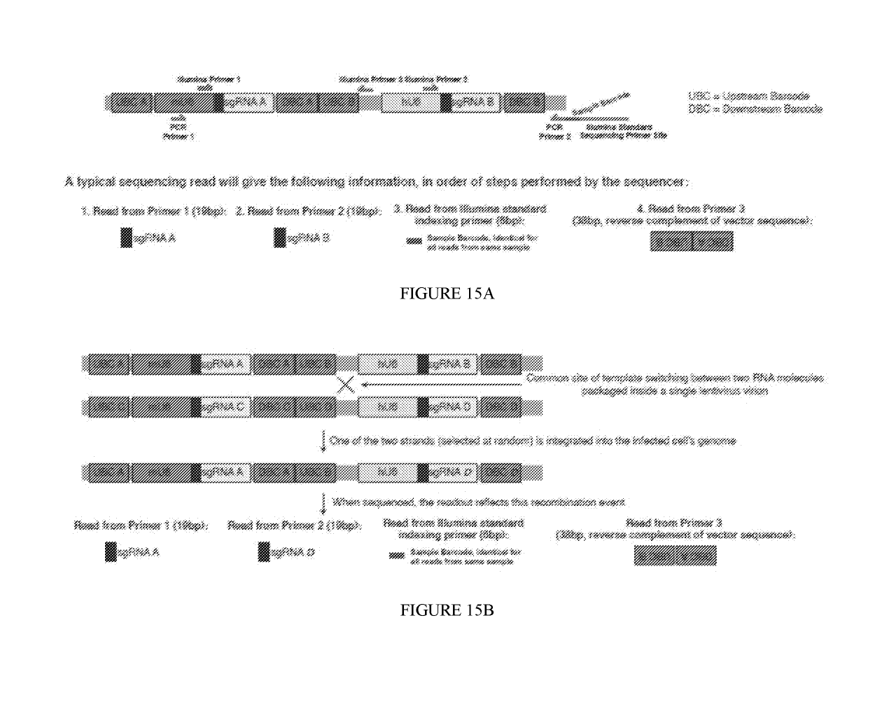

In the nucleic acid constructs provided herein, a pair of unique barcode sequences flank the polynucleotide sequence comprising the RNA polymerase III promoter operably linked to the nucleic acid encoding a small guide RNA (sgRNA) in each cassette. As shown in FIG. 15A, by flanking the polynucleotide sequence comprising the RNA polymerase III promoter operably linked to the nucleic acid encoding a small guide RNA (sgRNA) in each cassette with a pair of unique barcode sequences, the nucleic acid construct comprises a pair of adjacent barcode sequences flanked by a first and a second sgRNA, for example, sgRNA A and sgRNA B, respectively, as shown in FIG. 15B. The adjacent barcode sequences correspond to the downstream barcode sequence for the first sgRNA and the upstream barcode sequence for the second sgRNA.

By “adjacent” is meant that the barcode sequences are next to each other on the nucleic acid construct. In some examples, the barcode sequences are immediately adjacent to each other or separated by any number of nucleotides. For example, there can be about 1, 5, 10, 20, 30, 40, 50, 60, 70, 80, 90, 100, 200, 300, 400, 500 or more nucleotides in between the adjacent barcodes. In some examples, the pair of barcode sequences flanking each sgRNA have identical sequences and can range in length from about 10 to about 25 nucleotides. In other examples, the pair of barcode sequences flanking each sgRNA have different sequences and can range in length from about 10 to about 25 nucleotides. For example, the barcode sequences can be about 10, 11, 12, 13, 14, 15, 16, 17, 18, 19, 20, 21, 22, 23, 24 or 25 nucleotides in length. The unique barcode sequences serve as unique identifier sequences for each sgRNA. In some cases, the barcode sequences associated with each sgRNA are randomly assigned and unique. In some cases, the barcode sequences associated with each sgRNA are assigned by sequencing during library construction.

In the nucleic acid constructs provided herein, the length of the sgRNA is between about 85 to about 200 nucleotides. Therefore, the length of the sgRNA can be about 85, 90, 100, 110, 120, 130, 140, 150, 160, 170, 180, 190, 200, or any length in between these lengths. It is understood that the sgRNA does not have to be complementary to the entire target nucleic acid sequence as long as the gRNA can hybridize to the target nucleic acid in a site-specific manner. One can vary the length of complementarity in order to increase binding specificity and/or decrease offsite binding of the sgRNA.

The sgRNAs in the constructs provided herein can be selected to interact with any site-directed nuclease that requires a constant region of an sgRNA for function. These include, but are not limited to RNA-guided site-directed nucleases. Examples include, but are not limited to, nucleases present in any bacterial species that encodes a Type II CRISPR/Cas system. For example, and not to be limiting, the site-directed nuclease can be a Cas9 polypeptide, a C2c2 polypeptide or a Cpfl polypeptide. See, for example, Abudayyeh et al., Science 2016 Aug. 5; 353(6299):aaf5573; and Fonfara et al. Nature 532: 517-521 (2016).

In some examples, the site-directed nuclease is an enzymatically active site-directed nuclease, such as, for example, a Cas9 polypeptide. As used throughout, the term “Cas9 polypeptide” means a Cas9 protein or a fragment fragment thereof present in any bacterial species that encodes a Type II CRISPR/Cas9 system. See, for example, Makarova et al. Nature Reviews, Microbiology, 9: 467-477 (2011), including supplemental information, hereby incorporated by reference in its entirety. For example, the Cas9 protein or a fragment thereof can be from Streptococcus pyogenes. Full-length Cas9 is an endonuclease comprising a recognition domain and two nuclease domains (HNH and RuvC, respectively) that creates double-stranded breaks in DNA sequences. In the amino acid sequence of Cas9, HNH is linearly continuous, whereas RuvC is separated into three regions, one left of the recognition domain, and the other two right of the recognition domain flanking the HNH domain. Cas9 from Streptococcus pyogenes is targeted to a genomic site in a cell by interacting with a guide RNA that hybridizes to a 20-nucleotide DNA sequence that immediately precedes an NGG motif recognized by Cas9. This results in a double-strand break in the genomic DNA of the cell. In some examples, a Cas9 nuclease that requires an NGG protospacer adjacent motif (PAM) immediately 3′ of the region targeted by the guide RNA can be utilized. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to those described in Esvelt et al., Nature Methods 10: 1116-1121 (2013).

In some examples, the site-directed nuclease is a deactivated site-directed nuclease, for example, a dCas9 polypeptide. As used throughout, a dCas9 polypeptide is a deactivated or nuclease-dead Cas9 (dCas9) that has been modified to inactivate Cas9 nuclease activity. Modifications include, but are not limited to, altering one or more amino acids to inactivate the nuclease activity or the nuclease domain. For example, and not to be limiting, D10A and H840A mutations can be made in Cas9 from Streptococcus pyogenes to inactivate Cas9 nuclease activity. Other modifications include removing all or a portion of the nuclease domain of Cas9, such that the sequences exhibiting nuclease activity are absent from Cas9. Accordingly, a dCas9 may include polypeptide sequences modified to inactivate nuclease activity or removal of a polypeptide sequence or sequences to inactivate nuclease activity. The dCas9 retains the ability to bind to DNA even though the nuclease activity has been inactivated. Accordingly, dCas9 includes the polypeptide sequence or sequences required for DNA binding but includes modified nuclease sequences or lacks nuclease sequences responsible for nuclease activity. It is understood that similar modifications can be made to inactivate nuclease activity in other site-directed nucleases, for example in Cpfl or C2c2.

In some examples, the dCas9 protein is a full-length Cas9 sequence from S. pyogenes lacking the polypeptide sequence of the RuvC nuclease domain and/or the HNH nuclease domain and retaining the DNA binding function. In other examples, the dCas9 protein sequences have at least 30%, 40%, 50%, 60%, 70%, 80%, 90%, 95%, 98% or 99% identity to Cas9 polypeptide sequences lacking the RuvC nuclease domain and/or the HNH nuclease domain and retains DNA binding function.

In some examples the nucleic acid construct can be in a vector, such as a plasmid, a viral vector, a lentiviral vector, etc. In some examples, the nucleic acid construct is in a host cell. The nucleic acid construct can be episomal or integrated in the host cell. The compositions provided herein can be used to modulate expression of target nucleic acid sequences in eukaryotic cells, animal cells, plant cells, fungal cells, and the like. Optionally, the cell is a mammalian cell, for example, a human cell. The cell can be in vitro or ex vivo. The cell can also be a primary cell, a germ cell, a stem cell or a precursor cell. The precursor cell can be, for example, a pluripotent stem cell or a hematopoietic stem cell. Introduction of the composition into cells can be cell cycle dependent or cell cycle independent. Methods of synchronizing cells to increase a proportion of cells in a particular phase are known in the art. Depending on the type of cell to be modified, one of skill in the art can readily determine if cell cycle synchronization is necessary.

The compositions described herein can be introduced into the cell via microinjection, lipofection, nucleofection, electroporation, nanoparticle bombardment, and the like. The compositions can also be packaged into viral particles for infection into cells.

Also provided are cells including the compositions described herein and cells modified by the compositions described herein. Cells or populations of cells comprising one or more nucleic acid constructs described herein are also provided. For example, a cell comprising a first nucleic acid construct comprising multiple expression cassettes and a second nucleic acid construct comprising multiple expression cassettes, wherein the sgRNAs of the first nucleic acid construct and the sgRNAs of the second nucleic acid construct have different DNA targeting sequences, such that the sgRNAs of the first nucleic acid constructs target a first set of DNA targeting sequences and the second nucleic acid constructs target a second set of DNA targeting sequences are provided herein. For example, a cell comprising a first nucleic acid construct comprising two expression cassettes and a second nucleic acid construct comprising two expression cassettes, wherein the sgRNAs of the first nucleic acid construct and the sgRNAs of the second nucleic acid construct have different DNA targeting sequences, such that the sgRNAs of the first nucleic acid construct target two DNA sequences in the cell and the second nucleic acid constructs target two DNA sequences in the cell that are different from the two DNA sequences targeted by the sgRNAs of the first nucleic acid construct, are provided herein. In this way, modulation and identification of four target DNA sequences can be effected by multiple nucleic acid constructs. Thus, multiple nucleic acid constructs described herein can be used to target and modulate expression of four, five, six, seven, eight, nine, ten, eleven, twelve, thirteen, fourteen, fifteen, sixteen, seventeen, eighteen, nineteen, twenty or more DNA sequences. In some examples, expression of hundreds or thousands of target DNA sequences can be modulated.

As set forth above, each nucleic acid construct can comprise one or more expression cassettes encoding a reporter gene. Thus, a different reporter gene can be used for each construct, to individually track each nucleic acid construct in a cell or a population of cells. Cells include, but are not limited to, eukaryotic cells, animal cells, plant cells, fungal cells, and the like. Optionally, the cells are in a cell culture. Optionally, the cell is a mammalian cell, for example, a human cell. The cell can be in vitro or ex vivo. The cell can also be a primary cell, a germ cell, a stem cell or a precursor cell. The precursor cell can be, for example, a pluripotent stem cell or a hematopoietic stem cell. Introduction of the composition into cells can be cell cycle dependent or cell cycle independent. Methods of synchronizing cells to increase a proportion of cells in a particular phase are known in the art. Depending on the type of cell to be modified, one of skill in the art can readily determine if cell cycle synchronization is necessary.

### Methods

Described herein are methods of using nucleic acid constructs in CRISPR/Cas systems for modulating transcription of one or more DNA targets. These methods can be used to repress (CRISPRi), mutate (CRISPR) or activate (CRISPRa) all pairwise combinations of target genes identified in a primary CRISPR screen. The methods can be used to identify sgRNAs that target genes or genetic elements and produce a selected phenotype. The methods can also be used for small, medium, or large scale (e.g., genome-wide) screening of genetic elements that contribute to a selected phenotype. The methods can also be used to identify interacting genes and gene networks.

Provided herein are methods for sequencing constructs comprising barcoded pairs of sgRNAs in a cell. The methods generally involve sequencing the barcodes associated with the sgRNAs to identify cells that have not undergone a recombination event after introduction of the construct into the cell and eliminating cells that have undergone a recombination event after introduction of the construct into the cell. By eliminating cells that have undergone a recombination event, for example, in a CRISPR screen, analysis is done only those cells that have not undergone a recombination event, thus eliminating nonspecific interactions and background noise from genetic interaction mapping studies.

Described herein is a method for sequencing a first and a second sgRNA that target a first and a second DNA target in a genome of a cell, the method comprising: a) infecting a plurality of mammalian cells with a plurality of vectors to form a plurality of vector-infected cells, wherein each vector comprises: i) a first polynucleotide sequence comprising a first RNA polymerase III promoter operably linked to a nucleic acid encoding a first sgRNA comprising a sequence that targets a first DNA target in the genome and a first constant region that interacts with a site directed nuclease; and a pair of unique barcode sequences that flank the polynucleotide sequence comprising the RNA polymerase III promoter operably linked to the nucleic acid encoding the first sgRNA; and ii) a second polynucleotide sequence comprising a second RNA polymerase III promoter operably linked to a nucleic acid encoding a second sgRNA comprising a sequence that targets a second DNA target in the genome and a second constant region that interacts with a site directed nuclease; and a pair of unique barcode sequences that flank the polynucleotide sequence comprising the RNA polymerase III promoter operably linked to the nucleic acid encoding the second sgRNA; and b) expressing a site-directed nuclease in the mammalian cells; c) separating a selected pool of cells expressing a detectable phenotype from the plurality of infected cells; d) amplifying DNA comprising the nucleic acid encoding the first sgRNA and the nucleic acid encoding the second sgRNA in each cell with a pair of primers; wherein optionally, at least one of the primers includes a sample barcode sequence, and wherein the amplified DNA contains two adjacent barcodes flanked by the first and second sgRNAs; e) sequencing the nucleic acid encoding the first sgRNA and the nucleic acid encoding the second sgRNA in each cell; f) optionally sequencing the sample barcode; g) sequencing both adjacent barcode sequences to obtain a barcode sequence combination for each cell; h) comparing the barcode sequence combination obtained from each cell with the combination of the unique barcode sequence of the first sgRNA and the unique barcode sequence of the second sgRNA in the cell; and i) identifying the first and the second sgRNA that target a first and a second DNA target in cells comprising a combination of barcode sequences corresponding to the combination of the unique barcode sequence of the first sgRNA and the unique barcode sequence of the second sgRNA in the cell.

In some examples, the plurality of vectors comprises a library of dual-guide vectors, i.e., vectors comprising a first sgRNA and a second sgRNA targeting different DNA targets to identify interactions that cause a detectable phenotype. A library can comprise, at least 2 or more vectors. For example, a library can comprise at least 2, 3, 4, 5, 6, 7, 8, 9, 10, 20, 30, 40, 50, 60, 70, 80, 90, 100, 200, 300, 400, 500, 600, 700, 800, 900, 1000, 2000, 3,000, 4,000, 5,000, 6,000, 7,000, 8,000, 9,000, 10,000 or more dual guide-vectors. In other cases, using the formula N², where N is the number of unique sgRNAs, any number of unique sgRNAs can be used to create a dual-guide library. For example, 100 unique sgRNAs can be randomly combined into all sgRNA combinations generating a library of 10,000 dual guide combinations. In another example, 1000 unique sgRNAs can be randomly combined into all sgRNA combinations generating a library of 1,000,000 dual guide combinations.

In some examples, the first RNA polymerase III promoter and the second RNA polymerase III promoter in the vector have different sequences. In some examples, the first RNA polymerase III promoter and the second RNA polymerase III promoter in the vector are from different species. In some examples, the first constant region and the second constant region in the vector have different sequences. In some examples, the first constant region and the second constant region in the vector have the same or identical sequences

The method can be performed by contacting a plurality of mammalian cells with a plurality of vectors to form a plurality of vector-infected cells. In some examples, the vectors are lentiviral vectors that are packaged into viral particles for infection of cells. The multiplicity of infection can be controlled to ensure that the majority of the cells comprise no more than a single vector or a single integration event per cell.

In some examples, the plurality of cells is a heterogeneous population of cells (i.e., a mixture of different cells types) or a homogeneous population of cells. In some examples, the plurality contains at least two different cell types. In some examples, the cells in the plurality include healthy and/or diseased cells from a thymus, white blood cells, red blood cells, liver cells, spleen cells, lung cells, heart cells, brain cells, skin cells, pancreas cells, stomach cells, cells from the oral cavity, cells from the nasal cavity, colon cells, small intestine cells, kidney cells, cells from a gland, brain cells, neural cells, glial cells, eye cells, reproductive organ cells, bladder cells, gamete cells, human cells, fetal cells, amniotic cells, or any combination thereof.

In the methods provided herein, a site-directed nuclease is expressed in the mammalian cells. In some examples, the mammalian cells stably express a site-directed nuclease. In some examples, the site-directed nuclease is constitutively expressed. In some examples, the site-directed nuclease is under the control of an inducible promoter. In some examples, the mammalian cells are infected with a vector comprising a polynucleotide sequence encoding the site-directed nuclease prior to or subsequent to infecting the cells with the plurality of vectors. In any of the methods described herein, the site-directed nuclease can be transiently or stably expressed in the mammalian cells. In some examples, the site-directed nuclease is encoded by an expression cassette in the cell, the expression cassette comprising a promoter operably linked to a polynucleotide encoding the site-directed nuclease. In some examples, the promoter operably linked to the polynucleotide encoding the site-directed nuclease is a constitutive promoter. In other examples, the promoter operably linked to the polynucleotide encoding the site-directed nuclease is inducible. For example, and not to be limiting, the site-directed nuclease can be under the control of a tetracycline inducible promoter, a tissue-specific promoter, or an IPTG-inducible promoter.

The methods described can be used with any site-directed nuclease that requires a constant region of an sgRNA for function. These include, but are not limited to RNA-guided site-directed nucleases. Examples include nucleases present in any bacterial species that encodes a Type II CRISPR/Cas system. For example, and not to be limiting, the site-directed nuclease can be a Cas9 polypeptide, a C2c2 polypeptide or a Cpfl polypeptide. In some examples, the site-directed nuclease is the site-directed nuclease is an enzymatically active site-directed nuclease, such as, for example, a Cas9 polypeptide. In some examples, the site-directed nuclease is a deactivated site-directed nuclease, for example, a dCas9 polypeptide.

In some examples, the deactivated site-directed nuclease, for example, a deactivated Cas9, deactivated C2c2 or deactivated Cpfl polpeptide, is linked to an effector protein. Optionally, the site-directed nuclease is linked to the effector protein via a peptide linker. The linker can be between about 2 and about 25 amino acids in length. The effector protein can be a transcriptional regulatory protein or an active fragment thereof. The transcriptional regulatory protein can be a transcriptional activator or a transcriptional repressor protein or a protein domain of the activator protein or the inhibitor protein. Examples of transcriptional activators include, but are not limited to VP16, VP48, VP64, P192, MyoD, E2A, CREB, KMT2A, NF-KB (p65AD), NFAT, TET1, p300Core and p53. Examples of transcriptional inhibitors include, but are not limited to KRAB, MXI1, SID4X, LSD1, and DNMT3A/B. The effector protein can also be an epigenome editor, such as, for example, histone acetyltransferase, histone demethylase, DNA methylase etc.

The effector protein or an active fragment thereof can be operatively linked, in series, to the amino-terminus or the carboxy-terminus of the site-directed nuclease, for example, to dCas9. Optionally, two or more activating effector proteins or active domains thereof can be operatively linked to the amino-terminus or the carboxy-terminus of dCas9. Optionally, two or more repressor effector proteins or active domains thereof can be operatively linked, in series, to the amino-terminus or the carboxy-terminus of dCas9. Optionally, the effector protein can be associated, joined or otherwise connected with the nuclease, without necessarily being covalently linked to dCas9.

In the methods provided herein, once the cells have been infected, the cells are cultured for a sufficient amount of time to allow sgRNA:site-directed nuclease complex formation and transcriptional modulation, such that a pool of cells expressing a detectable phenotype can be selected from the plurality of infected cells

The phenotype can be, for example, cell growth, survival, or proliferation. In some examples, the phenotype is cell growth, survival, or proliferation in the presence of an agent, such as a cytotoxic agent, an oncogene, a tumor suppressor, a transcription factor, a kinase (e.g., a receptor tyrosine kinase), a gene (e.g., an exogenous gene) under the control of a promoter (e.g., a heterologous promoter), a checkpoint gene or cell cycle regulator, a growth factor, a hormone, a DNA damaging agent, a drug, or a chemotherapeutic. The phenotype can also be protein expression, RNA expression, protein activity, or cell motility, migration, or invasiveness. In some examples, the selecting the cells on the basis of the phenotype comprises fluorescence activated cell sorting, affinity purification of cells, or selection based on cell motility.

After selection of a pool of cells expressing a detectable phenotype, genomic DNA comprising the nucleic acid encoding the first sgRNA and the nucleic acid encoding the second sgRNA in each cell is amplified by polymerase chain reaction (PCR) with a pair of primers that bracket the genomic segment comprising the nucleic acid encoding the first sgRNA and the nucleic acid encoding the second sgRNA in each cell. In the methods provided herein, optionally, at least one of the PCR primers includes a sample barcode sequence that is added to the amplified DNA during amplification. The sample barcode sequence allows identification of all sequencing reads from the same sample, for example, when multiplexing multiple samples into single sequencing chip or lane. In the methods provided herein, the amplified DNA contains the first and second sgRNA sequences as well as the two adjacent barcodes that are flanked by the first and second sgRNAs (See FIG. 15A).

In any of the methods provided herein, individual cells from the pool or population of cells expressing a detectable phenotype can be placed into individual compartments. These compartments can be, but are not limited to, wells of a tissue culture plate (e.g., microwells) or microfluidic droplets. As used herein the term “droplet” can also refer to a fluid compartment such as a slug, an area on an array surface, or a reaction chamber in a microfluidic device, such as for example, a microfluidic device fabricated using multilayer soft lithography (e.g., integrated fluidic circuits). Exemplary microfluidic devices also include the microfluidic devices available from 10X Genomics (Pleasanton, Calif.).

In some examples, the cells are encapsulated in droplets. Relatively small droplets can be used in the methods provided herein. In some examples, the average diameter of the droplets may be less than about 5 mm, less than about 4 mm, less than about 3 mm, less than about 1 mm, less than about 500 micrometers, or less than about 100 micrometers. The “average diameter” of a population of droplets is the arithmetic average of the diameters of each of the droplets. In the methods provided herein, the droplets may be of the same shape and/or size, or of different shapes and/or sizes, depending on the particular application. In some examples, the individual droplets have a volume of about 1 picoliter to about 100 nanoliters.

A droplet generally includes an amount of a first sample fluid in a second carrier fluid. Any technique known in the art for forming droplets may be used. An exemplary method involves flowing a stream of the sample fluid containing the target material (e.g., cells expressing a detectable phenotype) such that the stream of sample fluid intersects two opposing streams of flowing carrier fluid. The carrier fluid is immiscible with the sample fluid. Intersection of the sample fluid with the two opposing streams of flowing carrier fluid results in partitioning of the sample fluid into individual sample droplets containing the target material. The carrier fluid may be any fluid that is immiscible with the sample fluid. An exemplary carrier fluid is oil. Optionally, the carrier fluid includes a surfactant or is a fluorous liquid. Optionally, the droplets contain an oil and water emulsion.

Oil-phase and/or water-in-oil emulsions allow for the compartmentalization of reaction mixtures within aqueous droplets. The emulsions can comprise aqueous droplets within a continuous oil phase. The emulsions provided herein can be oil-in-water emulsions, wherein the droplets are oil droplets within a continuous aqueous phase.

In some examples, a microfluidic device is used to generate single cell droplets, for example, a single cell emulsion droplet. The microfluidic device ejects single cells in aqueous reaction buffer into a hydrophobic oil mixture. The device can create thousands of droplets per minute. In some cases, a relatively large number of droplets can be generated, for example, at least about 10, at least about 30, at least about 50, at least about 100, at least about 300, at least about 500, at least about 1,000, at least about 3,000, at least about 5,000, at least about 10,000, at least about 30,000, at least about 50,000, or at least about 100,000 droplets. In some cases, some or all of the droplets may be distinguishable, for example, on the basis of an oligonucleotide present in at least some of the droplets (e.g., which may include one or more unique sequences or barcodes). In some cases, at least about 50%, at least about 60%, at least about 70%, at least about 80%, at least about 90%, at least about 95%, at least about 97%, at least about 98%, or at least about 99% of the droplets may be distinguishable.

In some examples, after the droplets are created, the device ejects the mixture of droplets into a trough. The mixture can be pipetted or collected into a standard reaction tube for thermocycling and PCR amplification. Single cell droplets in the mixture can also be distributed into individual wells, for example, into a multiwell plate for thermocycling and PCR amplification in a thermal cycler. After amplification, the droplets can be analyzed, for example, by sequencing, to identify sgRNAs and their corresponding unique barcodes in each single cell. In some cases, the cells are lysed inside the droplet before or after amplification. In other cases, the droplets can be distributed onto a chip for amplification. It is understood that numerous methods of generating droplets and amplifying nucleic acids therein are known in the art. See, for example, Abate et al., “DNA sequence analysis with droplet-based microfluidic,” Lab Chip 13: 4864-4869 (2013); and Kaler et al. “Droplet microfluidics for Chip-Based Diagnostics,” Sensors 14(12): 23283-23306 (2014)), both of which are incorporated herein in their entireties by this reference.

In any of the methods provided herein, droplets containing cells optionally may be sorted according to a sorting operation prior to merging with one or more reagents (e.g., as a second set of droplets). In some embodiments, a cell can be encapsulated together with one or more reagents in the same droplet, for example, biological or chemical reagents, thus eliminating the need to contact a droplet containing a cell with a second droplet containing one or more reagents. Additional reagents may include DNA polymerase enzymes, reverse transcriptase enzymes, including enzymes with terminal transferase activity, primers, and oligonucleotides. In some embodiments, the droplet that encapsulates the cell already contains one or more reagents prior to encapsulating the cell in the droplet. In yet other embodiments, the reagents are injected into the droplet after encapsulation of the cell in the droplet. In some embodiments, the one or more reagents may contain reagents or enzymes such as a detergent that facilitates the breaking open of the cell and release of the cellular material therein. Once the reagents are added to the droplets containing the cells, the DNA comprising the nucleic acid encoding the first sgRNA and the nucleic acid encoding the second sgRNA in each cell can be amplified in the droplet, for example, by polymerase chain reaction (PCR). Alternatively, the cells may be lysed in the droplet prior to amplification of the DNA comprising the nucleic acid encoding the first sgRNA and the nucleic acid encoding the second sgRNA.

In some cases, after thermocycling and PCR, the amplified products can be recovered from the droplet using numerous techniques known in the art. For example, ether can be used to break the droplet and create an aqueous/ether layer which can be evaporated to recover the amplification products. Other methods include adding a surfactant to the droplet, flash-freezing with liquid nitrogen and centrifugation. Once the amplification products are recovered, the products can be further amplified and/or sequenced.

The methods provided herein comprise sequencing the amplified DNA. Sequencing methods include, but are not limited to, shotgun sequencing, bridge PCR, Sanger sequencing (including microfluidic Sanger sequencing), pyrosequencing, massively parallel signature sequencing, nanopore DNA sequencing, single molecule real-time sequencing (SMRT) (Pacific Biosciences, Menlo Park, Calif.), ion semiconductor sequencing, ligation sequencing, sequencing by synthesis (Illumina, San Diego, Calif.), Polony sequencing, 454 sequencing, solid phase sequencing, DNA nanoball sequencing, heliscope single molecule sequencing, mass spectroscopy sequencing, pyrosequencing, Supported Oligo Ligation Detection (SOLiD) sequencing, DNA microarray sequencing, RNAP sequencing, tunneling currents DNA sequencing, and any other DNA sequencing method identified in the future. One or more of the sequencing methods described herein can be used in high throughput sequencing methods. As used herein, the term “high throughput sequencing” refers to all methods related to sequencing nucleic acids where more than one nucleic acid sequence is sequenced at a given time.

Any of the methods provided herein can optionally comprise deep sequencing of the amplified DNA. As used herein, “deep sequencing” refers to highly redundant sequencing of a nucleic acid. The redundancy (i.e., depth) of the sequencing is determined by the length of the sequence to be determined (X), the number of sequencing reads (N), and the average read length (L). The redundancy is then N×L/X. In the case of sgRNAs, the length of the sequence can be the length of the binding region, the full length of the sgRNA, or the length of a portion of the sgRNA that contains the binding region. The sequencing depth can be, or be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 110, 120, 130, 150, 200, 300, 500, 500, 700, 1000, 2000, 3000, 4000, 5000 or more. Deep sequencing can provide an accurate number of the relative frequency of the sgRNAs. Deep sequencing can also provide a high confidence that even sgRNAs that are rarely present in a population of cells (e.g., a population of selected test cells) can be identified.

As shown in FIG. 15A, once DNA is amplified from each cell, the nucleic acid encoding the first sgRNA and the nucleic acid encoding the second sgRNA are sequenced from the amplified DNA. In the methods provided herein, the first sgRNA and the second sgRNA can be sequenced from the same or opposite strand of amplified DNA. After sequencing the first and second sgRNA sequences, both adjacent barcode sequences are sequenced on the same or opposite strand of DNA to obtain a barcode sequence combination for each cell (see FIG. 15A). In some cases, the first sgRNA and the second sgRNA are sequenced from the same strand of amplified DNA and the adjacent barcode sequences are sequenced from the opposite strand of amplified DNA.

Optionally, the sample barcode is sequenced on the same or opposite strand of DNA, prior to or after sequencing the adjacent barcodes. The adjacent barcode sequences correspond to the downstream barcode sequence for the first sgRNA and the upstream barcode sequence for the second sgRNA.

The barcode sequence combination provides a unique combination sequence for the first and second sgRNA for each vector in each cell. This barcode combination is then compared with the combination of barcode sequences assigned to the first sgRNA (sgRNA A) and second sgRNA (sgRNA B) in the cell. As shown in FIG. 15A, if a combination of barcode sequences corresponding to or matching the combination of the unique barcode sequence of the first sgRNA (sgRNA A) and the unique barcode sequence of the second sgRNA (sgRNA B) is present in the cell in the cell, a recombination event has not occurred. Therefore, the cell can be reliably recorded as one cell containing a pair of sgRNAs that have not recombined with sgRNAs from other vectors in the plurality of vectors used to infect the plurality of mammalian cells. Once these cells have been identified, the DNA targets of the sgRNAs can be further analyzed to determine how and/or to what extent one or both of the DNA targets affect the phenotype. If a recombination event occurs, as shown in FIG. 15B, the adjacent barcodes will not correspond to the unique barcode sequences of the first sgRNA and the second sgRNA and these cells can be discarded to avoid convoluting the data. If only the adjacent barcodes shown in FIG. 15B were sequenced, one of skill in the art would have concluded that the cell contained the correct pair of sgRNAs. Alternatively, if only the two sgRNA sequences were sequenced, one of skill in the art would be unable to detect errors in the vector arising from recombination as well as inter-molecular recombination events introduced during the PCR step prior to sequencing.

The methods provided herein can further comprise identifying genetic interactions (GI) between the DNA targets targeted by each sgRNA of the pair. The ability to rapidly generate GI maps can identify previously unrecognized gene functions and inform the design of combination therapies based on synergistic pairs. For example, pairs of genes that exhibit synthetic lethality in cancer cells, but not healthy cells, are ideal targets for combination therapies aimed at limiting the emergence of drug resistance in rapidly evolving cells. As another example, if a first and a second gene form an unexpected synergistic genetic interaction for an undesirable phenotype (e.g., tumor growth), then a combination therapy that inhibits both targets can be designed.

In some examples, the GI map is a gain-of-function map constructed from a CRISPR-transcriptional activator (CRISPa) screen of sgRNA pairs. In some examples, the GI map is a loss-of-function map constructed from a CRISPR-transcriptional inhibitor (CRISPi) screen of sgRNA pairs.

Systematic genetic interaction (GI) maps have proven to be powerful tools for revealing gene functions within pathways or complexes (Pubmed IDs: 23394947, 14764870, 16487579, 20093466, 16269340, 17314980, 17510664, 24906158). A CRISPRa GI map or a combined CRISPRi/a GI map could yield rich novel biology elucidating how networks of proteins dictate cellular function (Pubmed ID: 21572441). More generally, quantitative methods of turning on and off one or multiple transcripts represents a critical tool for understanding how expression of the genes encoded in our genomes controls cell function and fate.

Disclosed are materials, compositions, and components that can be used for, can be used in conjunction with, can be used in preparation for, or are products of the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

The following examples are provided by way of illustration only and not by way of limitation. Those of skill in the art will readily recognize a variety of non-critical parameters that could be changed or modified to yield essentially the same or similar results.

### Example I

Functional genetic efforts typically face a tradeoff between complexity of perturbations (e.g., number of genes queried) and complexity of observed phenotype. Advances in pooled screening have made it possible to readily evaluate mammalian gene function at genome-scale, but to date have relied on simple phenotypic readouts that average properties of a population, such as the expression of a few exogenous reporters or cell viability. These approaches thus cannot distinguish mechanistically distinct perturbations that cause similar responses or when a bulk phenotype is driven by a subpopulation. These limitations underscore the need for high-content, single-cell screens at genome-scale.

The advent of droplet-based single-cell RNA-sequencing for profiling gene expression (Klein et al., 2015; Macosko et al., 2015; Zheng et al., 2016) has the potential to provide rich phenotypic data at the scale of hundreds of thousands of separately perturbed cells. To build a highly parallel platform for single-cell functional genomics, this technology was paired with CRISPR-based transcriptional interference (CRISPRi), which mediates gene inactivation with high efficacy and specificity (Qi et al., 2013; Gilbert et al., 2014; Horlbeck et al., 2016). Critically, a robust cell barcoding strategy that encodes the identity of the CRISPR-mediated perturbation in an expressed transcript, which is captured during single-cell RNA-seq analyses, was developed. This strategy, termed “Perturb-seq”, provides a readily implementable and scalable approach for parallel screening with rich phenotypic output from single cells. Moreover, a novel analytical pipeline to parse the massive datasets generated by Perturb-seq, which contain RNA-seq profiles of tens of thousands of individual cells, was developed. This approach successfully decomposes the noisy, high-dimensional single-cell data into a handful of more interpretable components, which enables decoupling of the responses to a given perturbation within individual cells and isolation of those responses from confounding effects, such as the cell cycle.

Here, Perturb-seq and its companion analytical pipeline were applied to the systematic analysis of the mammalian unfolded protein response (UPR). The UPR is an integrated endoplasmic reticulum (ER) stress response pathway that is coordinated by three distinct ER transmembrane sensor proteins (IRE1α, ATF6, and PERK). In response to various perturbations, including changes to protein folding capacity, calcium homeostasis, or membrane integrity, these sensors activate transcription factors (XBP1, the N-terminal cleavage product of ATF6, and ATF4, respectively) that promote survival or, when ER stress cannot be corrected, trigger cell death pathways (Walter and Ron, 2011). Briefly, IRE1α mediates noncanonical splicing of XBP1 mRNA to yield expression of the active XBP1 transcription factor (XBP1s). PERK is a kinase that, upon activation, phosphorylates the alpha subunit of the translation initiation factor complex EIF2 (eIF2α), which suppresses translation generally but paradoxically promotes translation of ATF4. Lastly, ATF6 is targeted to the Golgi where proteolytic cleavage releases a cytosolic transcription factor domain. Once activated, XBP1s, ATF4, and cleaved ATF6 translocate into the nucleus to initiate integrated, partially co-regulated programs of transcription. Considering the diversity of potential inputs and the complexity of outcome, comprehensive characterization of the UPR in mammalian cells requires both unbiased profiling of the physiological stresses that activate the sensors and delineation of the complex transcriptional phenotypes for each input.

To independently manipulate the three branches of the UPR, a programmable strategy for simultaneously repressing up to three genes with high efficacy was developed. Then, perturb-seq was used with combinatorial repression of the UPR sensor genes to delineate the distinct transcriptional programs of the three branches. Next, a two-tiered approach was used to interrogate the biological systems monitored by the UPR. Hundreds of genes that contribute to ER homeostasis were identified from two genome-wide CRISPRi screens and then Perturb-seq was applied to interrogate a diverse subset of these genes with single-cell resolution. These experiments allowed functional relationships between genes to be defined and allowed the complex, partially overlapping transcriptional responses to ER stress to be dissected. Furthermore, analysis of the single-cell responses revealed bifurcation of the UPR branches at two levels: among individual cells subject to the same perturbation and at the population level, where differential activation of the three UPR branches occurred across perturbations. The latter includes a dedicated feedback loop that enables a single arm of the UPR (the IRE1α/XBP1 branch) to specifically monitor expression of the protein translocation machinery. These data demonstrate the ability of Perturb-seq to provide rich biological insights and systematically dissect complex biological responses.

### Methods

Plasmid design and construction The perturb-seq expression vector (pBA439) was derived from a previously described CRISPRi expression vector (herein referred to as the “original CRISPRi expression vector”) (Addgene plasmid #60955) (Gilbert et al. 2014). To construct pBA439, the mU6-sgRNA-EF1a-PURO-BFP region from the original CRISPRi vector and a BGH polyadenylation sequence amplified by PCR from pcDNA3.1(+) (Invitrogen, V790-20) were inserted in reverse origination between the XbaI and EcoRI sites of the original CRISPRi expression vector. A random 18 nucleotide barcode was then inserted between the BFP and BGH polyA sequences (using disrupted EcoRI and AvrII sites) by Gibson assembly to construct the perturb-seq expression library (pBA571). The perturb-seq library was prepared with an estimated barcode diversity of >100,000 essentially as previously described (Kampmann, Bassik & Weissman 2014). Guide RNA protospacer sequences were individually cloned into both the original CRISPRi expression vector and the pBA571 library (between the BstXI and BlpI sites) by ligation. Each vector was then verified by Sanger sequencing of the protospacer and, if applicable, its corresponding barcode. Final guide expression vectors containing barcodes that introduced the conserved polyadenylation signal AATAAA (SEQ ID NO: 27) were discarded. To construct pMH0001, a minimal ubiquitous chromatin opening element (UCOE) (Müller-Kuller et al. 2015) was inserted upstream of the SFFV promoter in the lentiviral dCas9-BFP-KRAB expression vector (Gilbert et al. 2014).

The UPRE reporter was built into a backbone for lentiviral expression that has been previously described (Addgene plasmid #44012) (Meerbrey et al. 2011). This parental vector was digested with AgeI and religated to remove unwanted functional cassettes, and the UPRE promoter region or EF1a promoter were inserted between the BamHI and XhoI site of the resulting product. The UPRE promoter region contains 5 UPR elements (UPREs, 5′-TGACGTGG-3′ (SEQ ID NO: 28)) upstream of the c-fos minimal promoter (−53 to +45 of the human c-fos promoter) (Wang et al. 2000). Lastly, mCherry and sfGFP were cloned adjacent to UPRE and EF1α promoters, respectively (into an HpaI site). The resulting vectors are pBA407 (UPRE-mCh-Ubc-Neo) and pBA409 (EF1α-sfGFP-Ubc-Neo).

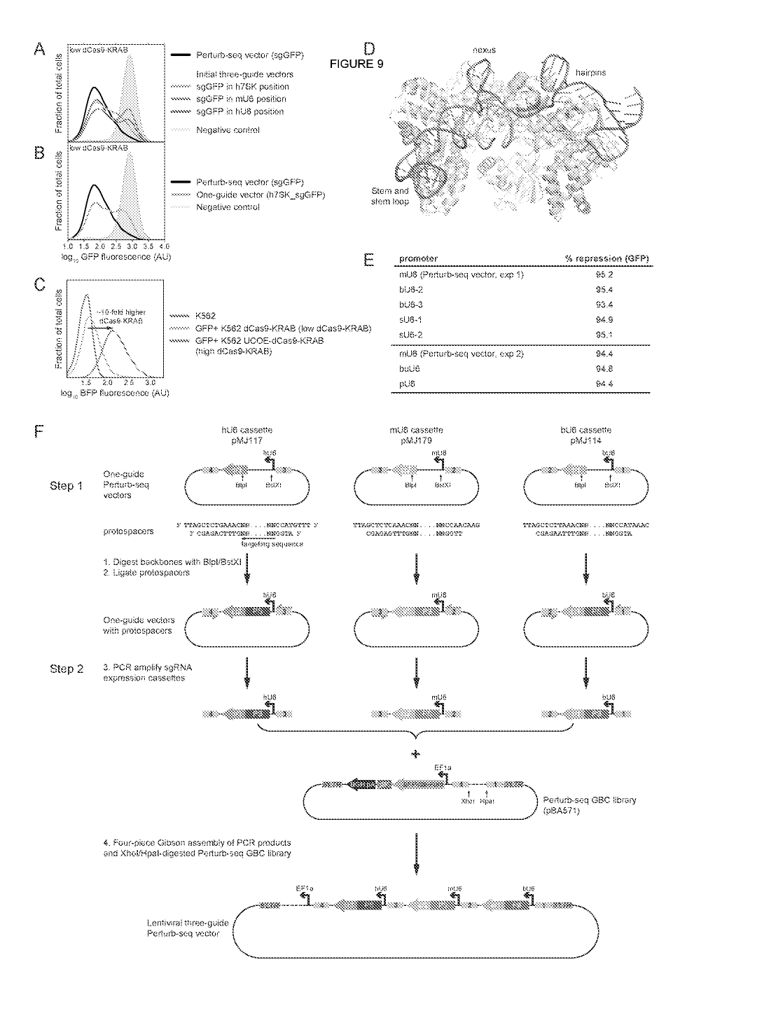

For testing of constant region variants in K562 cells, constant region variants fused to a GFP-targeting protospacer (EGFP-NT2, sequence GACCAGGATGGGCACCACCC (SEQ ID NO: 29)) or a negative control protospacer were PCRamplified and inserted into BstXI/XhoI-digested pBA439 (perturb-seq expression vector) by Gibson assembly. For testing of U6 promoters, U6 promoters from cow (bU6-2, GenBank DQ150531 and bU6-3, GenBank DQ150532 (Lambeth et al. 2006)), sheep (sU6-1, GenBank HM641427 and sU6-2, GenBank HM641426 (Hu et al. 2011)), buffalo (buU6, GenBank JN417659 (Zhang et al. 2014)), and pig (pU6, GenBank EU520423 (Chuang et al. 2009)) spanning ˜400-500 bp upstream of the TSS, modified to contain a BstXI site at the TSS, and fused to EGFP-NT2 and the original constant region were obtained as synthetic DNA segments (Integrated DNA technologies) and inserted into HpaI/XhoI-digested pBA439 (perturb-seq expression vector) by Gibson assembly. Three-guide vectors were assembled by a two-step cloning procedure (FIG. 9F). First, guide RNAs to be included were cloned into the corresponding single guide RNA expression vectors. Briefly, complementary oligonucleotides (Integrated DNA Technologies) containing the protospacer sequence and ligation overhangs were annealed and ligated into BstXI/BlpI-digested guide RNA expression vectors containing specific primer binding sites flanking the guide RNA expression cassette. The three-guide RNA expression cassettes were then PCR-amplified and assembled into HpaI/XhoI-digested pBA571 (perturb-seq expression library) by a single four-piece Gibson assembly step. Vectors were validated and barcodes were determined as described above. For three-guide vectors targeting the UPR branches, the bU6, mU6, and hU6 cassettes were designed to either express an sgRNA targeting ATF6, EIF2AK3 (PERK), or ERN1 (IRE1α), respectively, or a non-targeting negative control sgRNA. The following protospacer sequences were used: ATF6-targeting, gGGGATCTGAGAATGTACCA (SEQ ID NO: 30); EIF2AK3-targeting, gCGGGCTGAGACGTGGCCAG (SEQ ID NO: 31); ERN1-targeting, gAGAACTGACTAGGCAGCGG (SEQ ID NO: 32); non-targeting sgRNA in bU6 cassette, gACGACTAGTTAGGCGTGTA (SEQ ID NO: 33), non-targeting sgRNA in mU6 cassette, gGCCAAACGTGCCCTGACGG (SEQ ID NO: 34); non-targeting sgRNA in hU6 cassette, gCCTTGGCTAAACCGCTCCC (SEQ ID NO: 35).

Cell culture, DNA transfections, viral production, and construction of reporter cell lines K562 cells were grown in RPMI-1640 with 25 mM HEPES, 2.0 g/L NaHCO3, 0.3 g/L L-Glutamine supplemented with 10% FBS, 2 mM glutamine, 100 units/mL penicillin and 100 μg/mL streptomycin. HEK293T cells were grown in Dulbecco's modified eagle medium (DMEM) in 10% FBS, 100 units/mL penicillin and 100 μg/mL streptomycin. Cells were treated with tunicamycin or thapsigargin (Sigma, T9033) solubilized in DMSO. Lentivirus was produced by transfecting HEK293T with standard packaging vectors using TransIT®-LTI Transfection Reagent (Mirus, MIR 2306). Viral supernatant was harvested at least 2 days after transfection and filtered through a PVDF syringe filter and/or frozen prior to infection. To construct the UPRE reporter cell line, K562 cells stably expressing dCas9-KRAB (Gilbert et al. 2014), originally constructed from K562 cells obtained from ATCC 536 (RRID:CVCL_0004), were stably transduced with pBA407 and selected in media supplemented with 500 μg/mL Geneticin (Gibco, 10131-035). The clonal line cBA010 was then selected by limiting dilution. cBA011 is a derivative of cBA010 containing pBA409. cBA011 was made by stable transduction and selection of GFP positive cells using fluorescence activated cell sorting on a BD FACSAria2. The GFP reporter cell line 59 was constructed by infecting K562 cells stably expressing dCas9-KRAB with a Murine Stem Cell Virus (MSCV) retrovirus expressing GFP from the SV40 promoter. MSCV retrovirus was produced by transfecting amphotropic Phoenix packaging cell lines with standard packaging vectors. K562 cells stably expressing GFP were sorted to purity by flow cytometry using a BD FACS Aria2. To construct the GFP+ K562 UCOE-dCas9-KRAB cell line, the GFP reporter cell line was transduced with pMH0001 at a multiplicity of infection of ˜3. Transduced cells were sorted for BFP expression (top 33%) by flow cytometry on a BD FACS Aria2. BFP fluorescence was monitored for several generations and found to be stable.

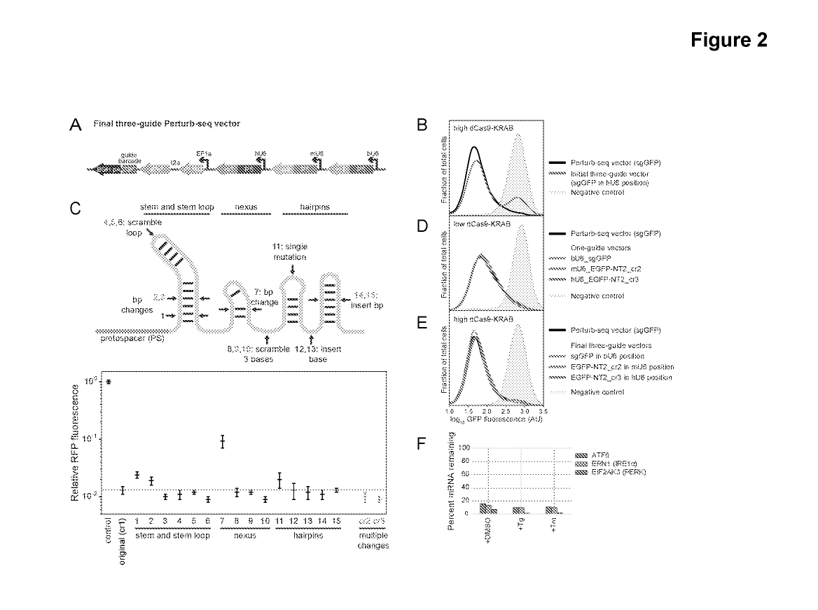

Design and cloning of constant region variants for testing in E. coli. Constant region bases to mutate were identified by inspection of the crystal structure of Cas9 bound to guide RNA and target DNA (FIG. 9D, PDB ID codes 4OO8, 4UN3, 4ZT0 (Nishimasu et al. 2014, Anders et al. 2014, Jiang et al. 2015)). Bases that did not form direct contacts with Cas9 or with other nucleotides of the constant region were deemed amenable for mutation. If applicable, sequence conservation patterns of the base in crRNAs/tracrRNAs of Streptococcus species and previous reports of constant regions carrying changes at the base (Briner et al. 2014, Dang et al. 2015) were used to determine the type of mutation. In this fashion, 15 constant region variants with mutations in different parts of the constant region were designed (FIG. 2D). The most diverse constant region variants cr2 and cr3 were designed by combining multiple individual mutations (FIG. 2D). To rapidly assess the activity of the variant constant regions, the variants were fused to a mRFP-targeting guide RNA (mRFP-NT1, sequence AACTTTCAGTTTAGCGGTCT (SEQ ID NO: 36)) (Qi et al. 2013) and tested in an E. coli CRISPRi reporter strain for knockdown of mRFP (see below). To eliminate variability from copy number variation, guide RNAs were cloned into a plasmid for site-specific integration into the E. coli genome at attL and expressed from single copy from an IPTG-inducible PLlacO-1 promoter (Lutz, Bujard 1997). To construct the integrating guide RNA expression plasmid, a guide RNA expression cassette was first PCR-amplified from pgRNAbacteria Addgene plasmid #44251) (Qi et al. 2013), modified to be flanked by the strong synthetic terminators L3S3P22 and L3S2P21 (Chen et al. 2013) and inserted into pCAH63 (Haldimann, Wanner 2001) at the ClaI/NheI sites. The constitutive promoter from pgRNA-bacteria was replaced with the IPTG-inducible PLlacO-1 promoter, generating pCs-550r. Then, pCs-550r was further modified to include the constant region used in mammalian CRISPRi (Gilbert et al. 2014), PCR-amplified with an mRFPtargeting protospacer and inserted into pCs-550r at the SpeI and KpnI sites to generate pMJ020. Finally, constant region variants 1-15 as well as cr2 and cr3 were cloned into pMJ020 by inverse PCR with mutations encoded in primer overhangs, by site-directed mutagenesis following standard procedures, or by insertion of a synthetic DNA segment encoding the constant region (Integrated DNA Technologies) into SpeI/KpnI digested pMJ020 by Gibson assembly.

Construction of E. coli CRISPRi reporter strain and testing of constant region variants The E. coli CRISPRi reporter strain was constructed by sequential insertion of a construct for IPTG-inducible expression of dCas9, a construct for constitutive expression of mRFP, and a construct for IPTG-inducible guide RNA expression (described above) into the E. coli genome. First, a lacIq-t0-PLlacO-1-dCas9 cassette (lacIq for strong expression of the Lac repressor; t0, a transcription terminator; PLlacO-1-dCas9; for IPTG-inducible expression of S. pyogenes D10A/H840A Cas9 (dCas9)) was inserted into the chromosome of E. coli BW25113 at +19 attL via of lambda Red recombinase mediated recombineering (Thomason et al. 2014). Then, a nfsA::mRFP-kan cassette for expression of mRFP from the J23119 promoter, a strong synthetic constitutive promoter from the Anderson promoter collection (http://parts.igem.org/Promoters/Catalog/Anderson), was inserted into an E. coli MG1655-derived strain by lambda Red recombinase-mediated recombineering as described previously (Qi et al. 2013), and moved from the MG1655-derived strain into the dCas9-expressing BW25113 strain by P1 transduction and selection on kanamycin following a published protocol (Thomason, Costantino & Court 2007). Plasmids for expression of mRFP-NT1 with the different constant region variants were integrated into the dCas9- and mRFP-expressing strain at attL using the helper plasmid pINT-ts (Haldimann, Wanner 2001), selecting for chloramphenicol resistance. Single colonies of strains with the integrated guide RNA expression plasmids were inoculated into LB and grown overnight in deep 96-well blocks at 37° C. with shaking at 900 rpm. Stationary-phase cultures were back-diluted 1:30 and grown into mid-exponential phase, at which point they were back-diluted 1:10000 into LB with 1 mM IPTG for induction of guide RNA and dCas9 expression. Induced cultures were grown at 37° C. with shaking until OD600 nm reached ˜0.4-0.7 (approximately 5 hrs), at which point they were diluted 1:30 in PBS in a 96-well plate. RFP fluorescence was recorded on a LSR-II flow cytometer (BD Biosciences) equipped with a 96-well high throughput sampler. Each experiment was carried out using three individual colonies for each constant region variant. RFP levels were normalized to those of a strain expressing a non-targeting guide RNA.

Testing of single- and three-guide vectors in K562 cells by GFP knockdown Vectors for expression of EGFP-NT2 in different contexts were delivered into GFP+ K562 cells with dCas9-KRAB or with UCOE-dCas9-KRAB by lentiviral transduction at MOI of 0.1-0.5. For all experiments using GFP+ K562 cells with UCOE-dCas9-KRAB, transduced cells were allowed to recover for 2 d, then selected to purity using 2 μg/mL puromycin for 3 d, and allowed to recover for another 2 d before GFP levels were recorded by flow cytometry on a LSR-II flow cytometer (BD Biosciences). For experiments involving only GFP+ K562 cells with dCas9-KRAB, cells were grown out for 7-9 d after transduction and GFP levels were recorded by flow cytometry, using BFP expression to gate for transduced cells. Flow cytometry data were analyzed using FlowCytometryTools v0.4.5 (http://eyurtsev.github.io/FlowCytometryTools/). For plotting, flow cytometry events were normalized to population size and the histograms were smoothened by kernel 62 density estimation. For estimating knockdowns, GFP levels of wild-type (GFP−) K562 cells were subtracted.

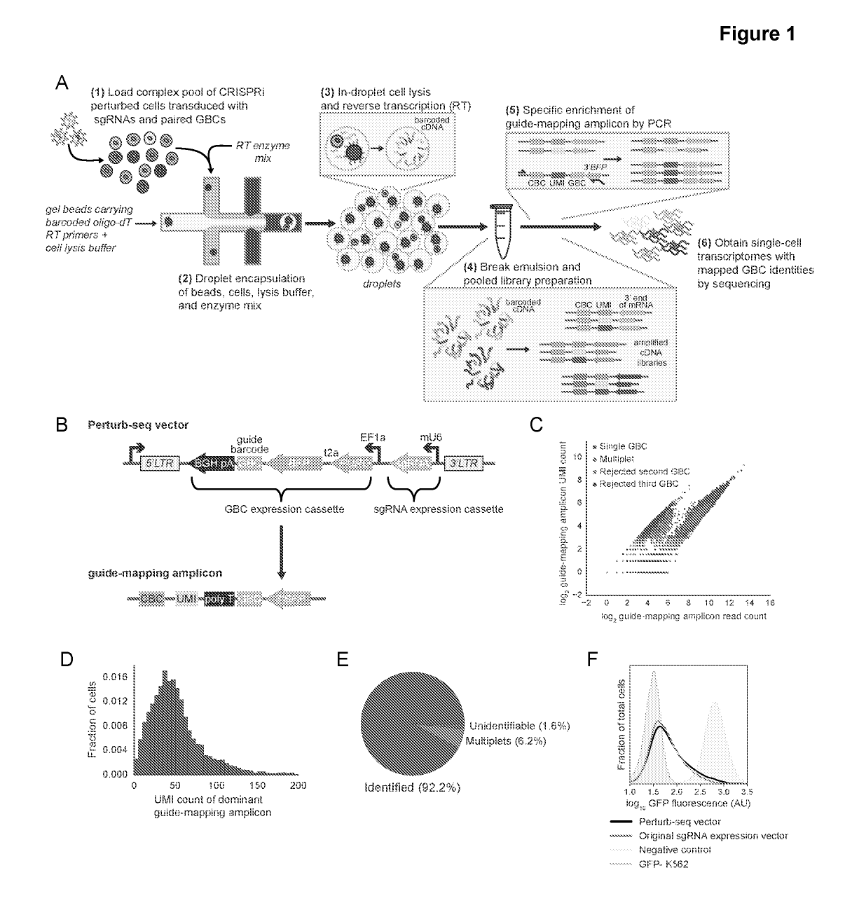

Perturb-seq screening Viruses were individually packaged and harvested in preparation for perturb-seq screening. Individual packaging of the lentivirus and pooling at the step of virus or cells was done to avoid intermolecular recombination of proviral genomes and to ensure maintenance of paired barcode-sgRNA coupling (Sack et al. 2016). For the pilot experiment represented in FIGS. 1C, 1D, 1F, cBA010 cells were individually spinfected with virus (at 33° C. for 2 hours at 1000×g) in media supplemented with 8 μg/mL polybrene; 5 hours post spinfection, virus was removed by centrifugation and cells were resuspended in fresh media. Three days later, a transduction efficiency of 20-30%, as determined by percentage of BFP positive (BFP+) cells, was measured by flow cytometry and cells were pooled with equal numbers of guide RNA-containing (BFP+) cells. Control cells were included in the pool at 3-fold coverage. Pooled cells were then grown in the presence of puromycin (3 μg/mL) for 5 additional days. Seven days post transduction cells were sorted on a BD FACSAria2 to near purity and eight days post transduction the sorted cells were separated into droplet emulsion using the Chromium™ Single Cell 3′ Solution according to manufacturer's instructions (10X Genomics).

For the perturb-seq epistasis experiment, seven three-guide vectors targeting every possible combination of ATF6, IRE1α, and PERK as well as two independent three-guide vectors with three negative control guide RNAs and different barcodes were individually packaged into lentiviruses. Freshly produced (i.e. not frozen) lentiviruses were spinfected into cBA007 cells (at 33° C. for 2 h at 1000×g) in media supplemented with 8 μg/mL polybrene. The virus was removed by centrifugation and cells were resuspended in fresh media. Three days after infection, transduction efficiencies of 5-10% were measured by flow cytometry. Cells were combined into a pool with equal numbers of transduced (BFP+) cells for each vector (resulting in 2-fold excess of negative control vectors) and the combined cells were then sorted on a BD 63 FACS Aria2 to near purity. To limit heterogenous effects of cell microenvironments caused by cell settling, the sorted cells were grown with continuous agitation on an orbital shaker. Five days after infection, the pooled and sorted cells were split into three populations, which were treated as follows: 1) DMSO control treatment for 6 hr; 2) treatment with 4 μg/mL tunicamycin for 6 hr; and 3) treatment with 100 nM thapsigargin for 4 hr. At the end of the treatment, the cells were separated into droplet emulsion using the Chromium™ Single Cell 3′ Solution according to manufacturer's instructions (10X Genomics). Cells loaded onto the device were 90.4%, 87.9%, and 85.3% viable for the different treatment conditions, respectively.

For the large-scale perturb-seq screen of UPR-inducing guide RNAs, viruses were individually titered by test infections into cBA011 cells and then pooled evenly. To account for varied effects on cell viability across the guide RNA sub-library and minimize cell number difference, pooling titers were determined by the percentage of BFP+ cells remaining 6 days post transduction. Two negative control guides were included, NegCtrl-2 and NegCtrl-3. NegCtrl-2 and select guides (those encoded by pDS002, pDS017, pDS026, pDS032, pDS033, pDS044, pDS052, pDS088, pDS091, pDS160, pDS186) were included at higher representation within the lentivirus pool, 8-fold and 2-fold, respectively. The lentivirus library pool was then used to infect cBA010 cells (performed by spinfection at 33° C. for 3 hours at 1000×g) so that a single pooled cell population with all perturbations would be carried though subsequent steps. Post centrifugation, cells were immediately removed from virus and transferred to a spinner flask for growth in fresh media. Three days later, a transduction efficiency of 15% was measured by flow cytometry and BFP+ cells were sorted to near purity on a BD FACSAria2. To limit heterogenous effects of cell microenvironments caused by cell settling, the sorted cells were grown with continuous agitation on an orbital shaker. Approximately seven days post transduction cells were separated into droplet emulsion using the Chromium™ Single Cell 3′ Solution across two separate runs totaling 10 lanes on the device according to manufacturer's instructions (10X Genomics). Cells loaded onto the device were 92% BFP+ and 93-94% viable, as determined by flow cytometry.

For all perturb-seq experiments single-cell RNA-seq libraries were prepared according to the Single Cell 3′ Reagent Kits User Guide (10X Genomics). However, this protocol produces libraries that are not compatible with the HiSeq 4000 due to the presence of some sort of toxic byproducts that it is uniquely sensitive to. To remove this issue, a short cleanup protocol, taking place after library preparation, was implemented. 120-200 ng of library material was split into parallel PCR reactions containing 0.3 μM each of the Illumina P5 and P7 primers, and amplified using Kapa HiFi ReadyMix according to the following protocol: (1) 95° C. 80 sec (2) 98° C. 20 sec/65° C. 30 sec/72° C. 20 sec for 6 cycles (3) 72° C. 1 min. PCR products were then SPRI-purified at 1× rati, repooled during elution, and then fragments of length 350-525 bp were selected using the BluePippin (Sage Science).

Specific amplification of guide barcodes Parallel PCR reactions were constructed containing 30 ng of final library as template, 0.6 μM PTMN050-P7, and 0.6 μM barcoded PTMN051, and amplified using Kapa HiFi ReadyMix according to the following PCR protocol: (1) 95° C. 3 min (2) 98° C. 15 sec/70° C. 10 sec for 14-16 cycles. Reactions were repooled during 0.8× SPRI selection, and then fragments of length 350-425 were selected using the BluePippin.

Genome-scale CRISPRi screening Reporter screens were conducted using protocols similar to those previously described (Gilbert et al. 2014, Sidrauski et al. 2015). The hCRISPRi-v1 (Gilbert et al. 2014) or the compact (5 sgRNA/gene) hCRISPRi-v2 (Horlbeck et al. 2016) sgRNA libraries were transduced into cBA011 cells at an MOI <1 (percent BFP+ cells was ˜45% and 26%, respectively). For the hCRISPRi-v1 screen, cells were grown in spinner flasks for 2 days without selection, followed by 3 days of selection with 1 μg/mL puromycin. Screen replicates were split post infection and carried separately throughout the remainder of the experiment. One replicate arm of the hCRISPRi-v1 screen was carried with media supplemented with 88-150 nM ISRIB throughout, although differences observed between the replicates at the level of both 65 sgRNAs and genes were negligible. For the hCRISPRi-v2 screen, cells were grown in spinner flasks for 2 days without selection, followed by 5 days of selection with 1-3 μg/mL puromycin. Screen replicates were split into separate spinner flasks on day 3. For both screens, cells were separated into those with the highest (˜28-33%) and lowest (˜30-35%) mCherry/GFP ratio eight days post transduction by fluorescence-activated cell sorting. Cell pellets were frozen after collection. Approximately 23-30 million cells were collected per bin during screening of the hCRISPRi-v1 library (a representation of ˜450) and 19-22 million cells per bin for hCRISPRi-v2 (a representation of ˜600). Genomic DNA was isolated from frozen cells and the sgRNA-encoded regions were enriched, amplified, and prepared for sequencing. hCRISPRi-v2 was sequenced with greater coverage.

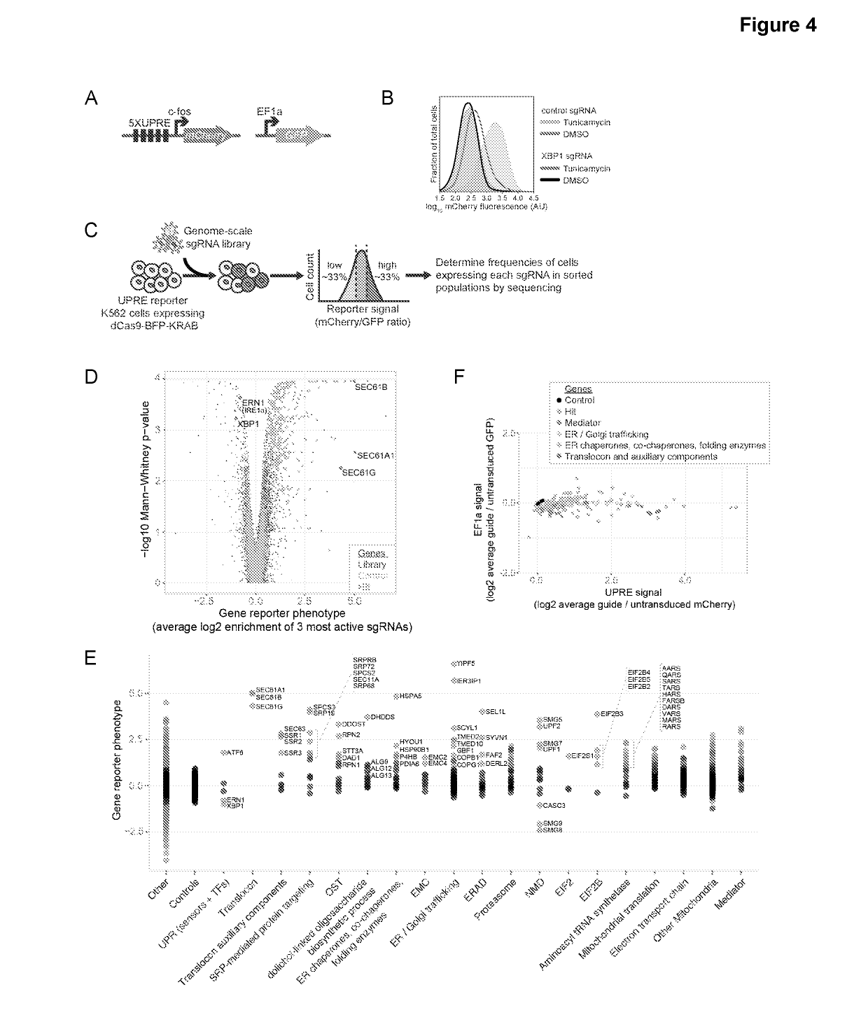

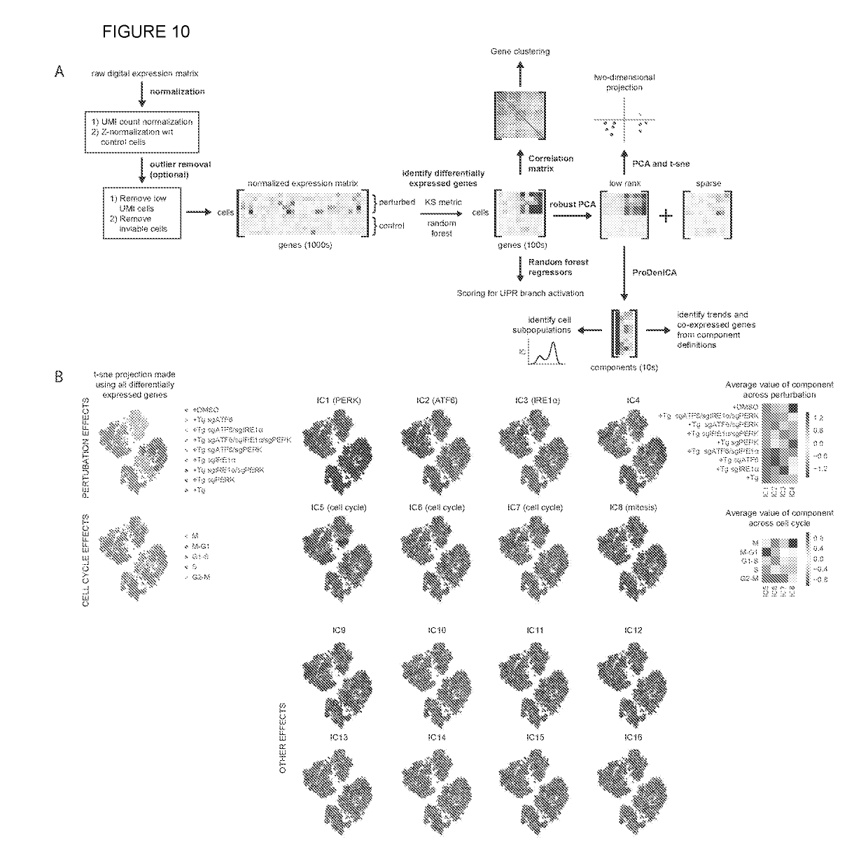

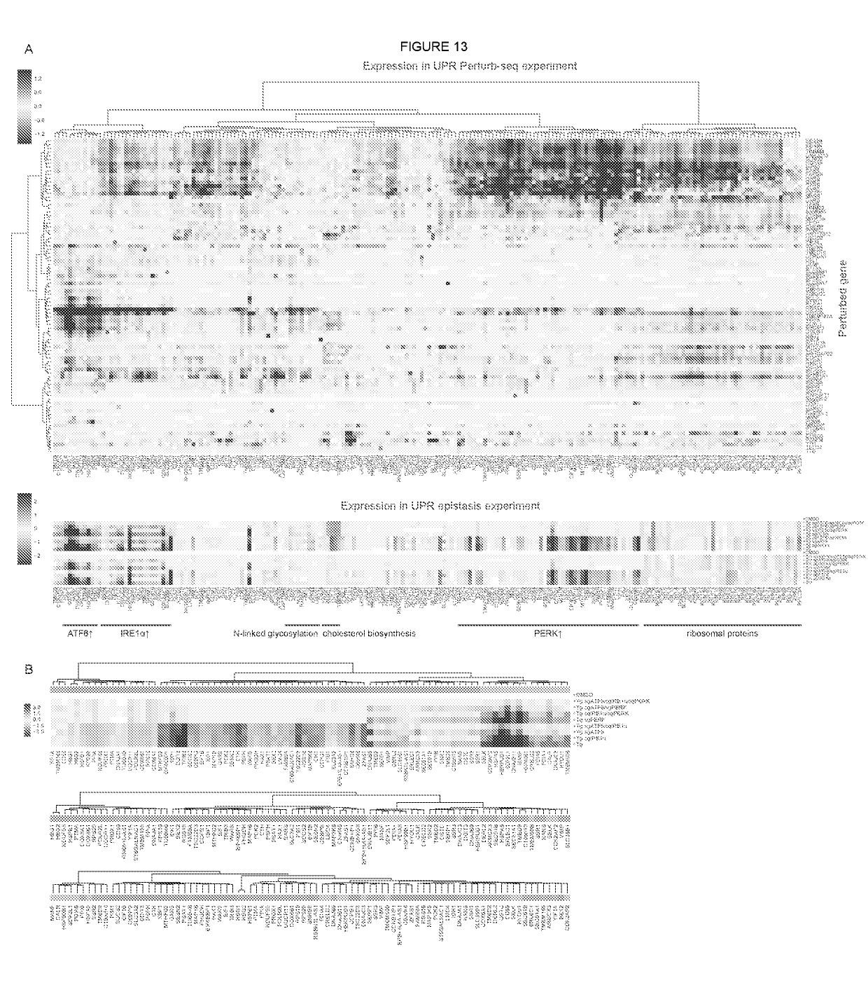

Sequenced protospacer sequences were aligned and data were processed as described (Gilbert et al. 2014, Horlbeck et al. 2016) with custom Python scripts (available at https://github.com/mhorlbeck/ScreenProcessing). Reporter phenotypes (referred to as Reporter signal) for library sgRNAs were calculated as the log2 enrichment of sgRNA sequences identified within the high mCherry/GFP cells over the low mCherry/GFP cells. Phenotypes for each transcription start site were then calculated as the average reporter phenotype of the 3 sgRNAs with the strongest phenotype by absolute value (most active sgRNAs). Mann-Whitney test p-values were calculated by comparing all sgRNAs targeting a given TSS to the full set of negative control sgRNAs. For data presented in FIGS. 4D-F and 10B, C, genes with multiple targeted TSSs were collapsed such that only the TSS with the lowest p-value was used. Screen hits were defined as those genes (or separately those TSSs) with a discriminate score, defined as the absolute value of a calculated reporter phenotype over the standard deviation of all evaluated phenotypes multiplied by the log 10 of the Mann-Whitney p-value for given candidate, greater than 7. Growth screen data in FIGS. 10C and 13C has been reported elsewhere, and the second, unreported screen was conducted in parallel essential as described (Horlbeck et al. 2016). Gene ontology analysis was conducting using select databases (GOTERM_BP_FAT, 66 GOTERM_CC_FAT, GOTERM_MF_FAT, KEGG_PATHWAY) and hits (calculated from all TSSs) with a phenotype of greater than 1 using DAVID Bioinformatic Resources 6.8 Beta (https://david-d.ncifcrf.gov/) with (Huang, Sherman & Lempicki 2009a, Huang, Sherman & Lempicki 2009b). Biological classifications reported in FIG. 4F-G were manually assembled from the literature and using resources from the HUGO Gene Nomenclature Committee (www.genenames.org), AmiGO, the GO Consortium's annotation and ontology toolkit (Carbon et al. 2009) (http://amigo.geneontology.org), DAVID Bioinformatic Resources (https://david.ncifcrf.gov) (Huang, Sherman & Lempicki 2009a, Huang, Sherman & Lempicki 2009b). The gene sets defining these classifications are listed in.

Individual evaluation of sgRNA reporter phenotypes Viruses were individually packaged and harvested as described above. UPRE reporter-containing K562 cells (cBA011) cells were infected with thawed virus. Additionally, parental K562 dCas9-KRAB cells (Gilbert et al. 2014) were transduced with negative controls. Flow cytometer readings of the mCherry UPRE signal and GFP EF1a signal were taken periodically and 8 days post transduction. Median fluorescence signals were analyzed by subtracting an average background signal from control-transduced K562 dCas9-KRAB cells and normalizing the mCherry, GFP, or mCherry/GFP measurement from guide-containing cells (as determined by BFP fluorescence) in each well to untransduced cells. Data from wells with fewer than 500 transduced or untransduced cells or with lower than expected BFP signal (3 standard deviations below the mean of BFP median from all other experimental wells) were systematically discarded from further analysis. For experiments where a flow cytometer reading was taken on the second day post transduction, data was also filtered for a minimum day 2 viability percentages.

RT-qPCR and semi-quantitative PCR for XBP1 mRNA splicing Cells were harvested and total RNA was isolated using TRIzol® Reagent (ThermoFisher Scientific, 15596-018) and Phase Lock Gel tubes (VWR, 10052-170) or NucleoSpin® RNA (Macherey-Nagel, 67 740955.50) essentially according to manufacturers' instructions. RNA prepared by TRIzol® extraction was treated with TURBO™ DNase (ThermoFisher Scientific). RNA was converted to cDNA using SuperScript® II or SuperScript® III Reverse Transcriptase (ThermoFisher Scientific) under standard conditions with oligo(dT) primers or random hexamers with or without RNaseOUT™ Recombinant Ribonuclease Inhibitor (ThermoFisher Scientific). Quantitative PCR reactions were prepared with 1× master mix containing 1× Colorless GoTaq® Reaction Buffer (Promega, M792A), MgCl2 (0.7 mM), dNTPs (0.2 mM each), primers (0.75 μM each), and 1000× SYBR Green with GoTaq® DNA polymerase (Promega, M830B) in 22 μL reactions. Reactions were run on a LightCycler® 480 Instrument (Roche). Semi-quantitative XBP1-specific PCR reactions were prepared with 2 μL of cDNA diluted 1:10 using a master mix containing 0.9× Colorless GoTaq® Reaction Buffer (Promega, M792A), dNTPs (0.23 mM each), primers (0.45 μM each) with GoTaq® DNA polymerase (Promega, M830B) in 22.1 μL reactions. These reactions were run on a standard thermocycler program with 30 second at 60.5° C. for annealing and 28 cycles. PCR products were visualized on 8% TBE gels. Primers used can be found in.

Quantification and Statistical Analysis

The following provides an overview of the methods used and their specific application to each figure provided herein.

Pipeline overview All analysis was performed in Python, using a combination of Numpy, Pandas, scikit-learn, and a custom-made perturb-seq library. The general outline is presented in FIG. 9A and the steps are outlined below.

Sequencing: Reads from 10X single-cell RNAseq experiments were aligned and collapsed to unique molecular identifier (UMI) counts using 10X's cellranger software 68 (version 1.1). The result is a large digital expression matrix with cell barcodes as rows and gene identities as columns.

Perturbation identity mapping Specifically amplified guide barcode libraries were described as above and either sequenced as spike-ins or independently. The specific amplification strategy used preserved the 3′ end of the transcript (and thus the cell barcode and UMI of a given captured molecule) and introduced an Illumina read 1 primer upstream of the guide barcode sequence. These reads were aligned using bowtie (flags: -v2-q-m1) to a library of expected barcode sequences. All reads with common cell barcode, UMI, and read identity (as some reads were not mapped by bowtie due to low quality scores) were collapsed to produce a table consisting of possible guide identities for each cell, and the number of molecules attributing a given guide identity to that cell. The coverage of a given proposed identity was defined as the number of reads divided by the number of UMIs, and defined a proposed identity as having good coverage if it: (1) had a coverage level at or above the mean coverage level minus two times the standard deviation in coverage (2) had at least 50 raw reads and (3) had at least 3 UMIs. Any cell that had only a single identity that met these criteria was assigned that perturbation identity. Any cell that had two or more identities meeting these criteria was assigned as a multiple (either a multiple infection, or a multiple encapsulation during emulsion generation). Any cell that had no identities meeting these criteria was assigned as unidentifiable.

Expression normalization To normalize for differences in sequencing capture and coverage across emulsion droplets, all cells were rescaled to have the median number of total UMIs (i.e. each row of the raw digital expression matrix is normalized to the same sum). Expression of each gene was then z-normalized with respect to the mean and standard deviation of that gene in the control (unperturbed) population:

$${x}_{normalized} = \frac{x - {μ}_{control}}{{σ}_{control}}$$

This normalization means that control cells always have mean normalized expression of 0 for all genes and standard deviation 1, so that the units of expression are “standard deviations above/below the control distribution.” In the epistasis experiment, the control population was the DMSO-treated cells. In the perturb-seq experiment, they were the cells containing the NegCtrl-2 guide. In the perturb-seq experiment, the mixed population was run in ten separate pools that were treated independently during library preparation (corresponding to lanes on the 10X Chromium instrument and on the Illumina sequencer). To avoid any lane dependent batch effects, cells were normalized to the control cell distribution within the same lane.

Low cell count/inviable cell removal While developing the low rank ICA method described below, it was observed that all experiments always contained two subpopulations that were peculiar in that they contained roughly equal membership from all perturbations. Further investigation showed that these were a group of cells with systematically lower total UMI counts (visible as a small second mode in the distribution of total UMIs per cell) and a group of cells that contained markers of activation of apoptotic programs. The first population was attributed to partly failed RNAseq library preparation occurring in a small number of emulsion droplets, and the second to inviable cells (which we knew were present in the cells placed used in the 10X experiment). Neither population composed more than a few percent of the total number of cells. Though low rank ICA always isolated these in an unbiased way, these were generally excluded from analysis. The low UMI count cells were simply removed using a threshold. To remove the apoptotic cells, a random forest regressor (described in more detail below in the section on UPR branch activation scoring below) was trained to recognize them using the cells in our epistasis experiment as training data.

Identification of differentially expressed genes The end result of the previous steps is a normalized gene expression matrix where each cell has been assigned a perturbation identity. In general, analyzing differences between populations was of interest, and two distinct strategies for isolating interesting genes were used. Kolmogorov-Smirnov test/metric: The Kolmogorov-Smirnov test is a nonparametric test for equality of probability distributions based on a metric defined on their cumulative distribution functions. Specifically, if Fperturbed and Fcontrol are the CDFs for a given gene in the perturbed and control distribution, the test statistic is

$$D = {sup}_{X} \vert {F}_{perturbed} ( x ) - {F}_{control} ( x ) \vert$$

This can be assigned a p-value in a standard way. However, the large scale of single-cell data means that many genes were often significantly perturbed without being interestingly perturbed, simply because of small differences detected by great sampling depth. Thus, in some examples a direct threshold was placed on the test statistic D itself, which ensured that changes were both significant (in the statistical sense) and also of reasonable magnitude, as it is valid metric on the space of CDFs.

Random forest classifier An advantage of perturb-seq is that the cell populations are known, which means that supervised learning methods can be brought to bear. The strategy here was motivated by the idea that a gene is likely important for a given perturbation if its expression level can be used to accurately predict that perturbation's identity. This idea is particularly useful when many perturbations are being compared, as what you want then are the genes that best distinguish all of the perturbations from each other. To leverage this idea, random forest classifiers were used. Given a set of perturbations, a random forest classifier was trained to predict perturbation identity using a subset of genes. Specifically, the implementation of extremely randomized trees implemented in scikit-learn was used, generally with 1000 trees in the forest. A two-stage fitting process was performed for a given number of desired features N genes. First, 20% of the cells were set aside. The remaining 80% were used to train a random forest classifier (usually with 1000 estimators) to predict the perturbation identity using the normalized expression profile for each cell (with some threshold on gene expression level) as the set of features. The random forest assigns importances to features during training based on their predictive value, and we would then take the top Ngenes sorted by importance as the set of most informative genes. To evaluate how informative these genes were, the classifier was then retrained using only these genes, and the perturbation present in the 20% of cells that had initially been set aside was predicted. For sets of perturbations with large differences, accuracies of 80-90% were routinely seen. The genes chosen by the random forest essentially always showed marked differences by the Kolmogorov-Smirnov approach outlined above, and the forests had the advantages that they scaled to an arbitrary number of perturbations, and that the selected genes were known to vary informatively across perturbations instead of simply having a difference in distribution.

Low rank ICA Single-cell data are intrinsically very noisy, either due to real biological variation or problems in capture efficiency. As described in the main text, these effects can affect the sensitivity of methods like principal components analysis, which is intrinsically variance-maximizing and hence very sensitive to outliers. To isolate larger trends within the data, a simple two-step approach called low rank ICA was developed. The first step consists of isolating a low rank approximation of the dynamics within the experiment. To do this, Robust PCA (Candès et al. 2011), which seeks a decomposition of the form

X=L+S

where X is the normalized expression matrix, L is a low rank matrix, and S is a sparse matrix (most entries are zero) was used. Specifically, Robust PCA solves the optimization problem

$${min}_{L , S} {‖ L ‖}_{*} + λ {‖ S ‖}_{1} subject to X = L + S$$

where ∥·∥* is the nuclear norm (sum of singular values) and ∥·∥ is the sum of the absolute values of the entries of the matrix. These constraints naturally induce L to be low rank, and S to be sparse. In implementations, the augmented Lagrangian multiplier method (Lin, Chen & Ma 2010), which was fast and efficient, was used.

It should be noted that the interpretation of this optimization problem is slightly different from that seen in some other instances, where S is regarded as capturing noise corrupting the “true” dynamics seen in L. In single-cell data the “noise” may actually be biological in origin, but our primary intent is to isolate the low rank approximation L, which is effectively a smoothed version of the population's dynamics that leaves major trends intact. The advantage of the decomposition of course is that the S matrix is still available afterward, and it may in fact carry useful information about highly stochastic processes within the population.

The next goal was to isolate the major trends within the low rank dynamics of the population. To do this independent components analysis (ICA) was applied. ICA posits a model in which the expression of a given gene (yj) can be decomposed as a linear sum of various effects (s1 to sn) that are statistically independent of each other:

yⱼ=aⱼs₁+aⱼ₂s₂+ . . . +aⱼₙsₙ

Solving this problem is beyond the scope of this section, but interest lies primarily in the vector version of this formula,

y=As

in which a cell's expression profile y (over all genes) is viewed as a linear sum of independent effects, and the equivalent matrix version

Y=AS

in which all of the dynamics of the cells within the population (the columns of Y) was decomposed into sums of independent components (ICs). The matrix A above is called the mixing matrix, and in this context describes which genes contribute to which effects. As noted above, the key difference in this case, from decompositions like principal components analysis, is that the s components are derived in a way to make them as statistically independent as possible. Once the matrix A is estimated, the dynamics of each cell in the population can be “unmixed” by applying the inverse operation (denoted here by W) to its expression profile:

s=Wy

This yields a low-dimensional description of what each cell is doing in terms of the independent factors given by s. In this case ICA was applied to the low rank matrix L, i.e. Y=LT above. Thus, an attempt was made to separate the population's low rank dynamics into independent factors. As the ICA minimization problem posed in the strongest form cannot practically be solved, different algorithms will give somewhat different answers based on the tradeoffs they make. After trying several methods, the ProDenICA algorithm (Friedman, Hastie & Tibshirani 2001), which was found to frequently give the highest quality components, was used.

In general low rank ICA is applied in two ways. First, it can be used to partition cells into subpopulations. Strong trends often lead to independent components that are bimodal, so simply thresholding the value of a component is a means of clustering. However, an advantage of this method of subpopulation identification is that it can also identify continuous trends, rather than enforcing discrete categories that may not exist like in other methods of clustering. Secondly, the mixing matrix A is very informative, as it determines the extent to which each gene contributes to a given component. This can be useful both in understanding what the component is measuring (if the most heavily weighted genes have a clear common function) and in identifying groups of genes that are co-expressed in an unbiased way. Interpretation of independent components does have some caveats. First, they have no natural sign (so an “enriched” effect may appear as a low value of an independent component) or scale: thus there is no natural order where the first IC is somehow more informative than the next, consistent with the fact that they are meant to represent independent effects. One pragmatic solution is to order the components by the norm of the corresponding column in the mixing matrix, which tends to place the most interesting components first.

t-sne visualization To obtain two-dimensional projections of the population's dynamics, the dimensionality of the low rank matrix L using classical PCA (with the number of components determined from a scree plot) is reduced, and then these components are further reduced via t-distributed stochastic neighbor embedding. Occasionally, ICs are directly visualized in this way as well, but because they lack intrinsic scale like principal components, dominant effects can be crowded out by minor ones.

Hierarchical clustering of genes Several of the analyses described herein use single-cell coexpression information to cluster genes. For a given list of genes, this clustering is performed by first calculating the gene-gene correlation matrix ρ over all cells in the population. This is then converted to a dissimilarity matrix π via the transformation π=√2(1−ρ). The dissimilarity matrix is then clustered using Ward's method. For visualization purposes, the optimal leaf ordering algorithm in MATLAB is applied. This reorders the leaves in the dendrogram by flipping tree branches to maximize the similarity between adjacent leaves, but without dividing any branches (i.e. the clustering is unchanged, but the dendrogram ordering is in some sense optimal). We then reorder the columns and rows of the correlation matrix via the resulting ordering, so that groups of genes with correlated expression appear as blocks along the diagonal.

Cell cycle position An approach previously described, in which the expression of sets of experimentally-derived genes specific for each cell cycle phase is used for each cell to score cell cycle phase (Macosko et al. 2015), was used.

Average expression profiles Synthetic bulk profiles are often created for different populations. These are created by averaging the normalized expression profile of each cell within that population together.

Analytical steps for each figure The analysis behind each figure provided herein is described below.

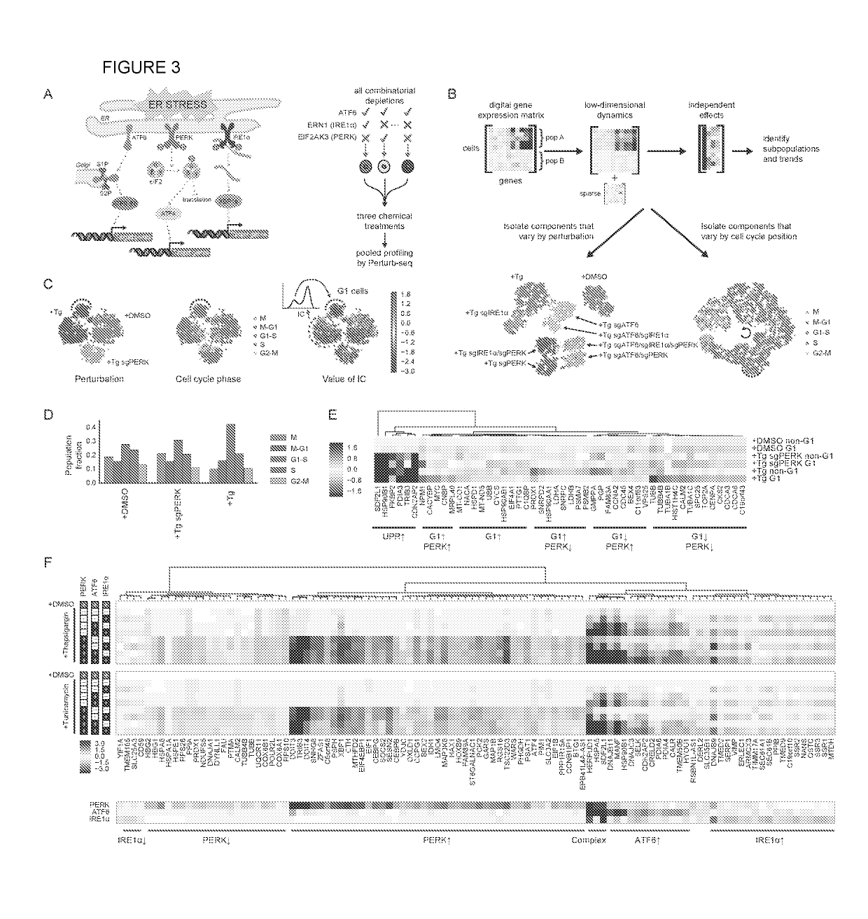

Single-cell analysis in FIG. 3 A population consisting of cells treated with 100 nM thapsigargin in each of our 8 genetic backgrounds was formed, along with DMSO-treated control cells. As outlined in the “Low cell count/inviable cell removal” section, cells with substantially lower than average UMI counts or that scored strongly for inviability markers from analysis were removed, as these groups partitioned away from the rest of the population in preliminary analyses. For each perturbation, genes that were differentially expressed relative to the control were identified, as described in the “Identification of differentially expressed genes.” A list of all genes that had a mean expression of at least 0.5 UMI per cell in the population and for which the Kolmogorov-Smirnov test statistic D>0.15 in at least one perturbation was made. This led to a group of 1,711 differentially expressed genes. A reduced gene expression matrix containing only these genes was formed, and low rank ICA was performed to reduce the population's dynamics therein to 16 ICs (FIG. 10B). The raw trends in the population were examined by reducing the low rank matrix to 16 components via PCA (16 components) and then to two dimensions via t-sne, revealing a general breakdown by perturbation and by cell cycle within each perturbation (FIG. 10B). Then, ICs whose average value varied either across the perturbation, or across the cell cycle position, were identified. For each category, four components showed clear trends at the average level and in the t-sne plots (FIG. 10B). For example, several of the components clearly showed the expected epistasis patterns for PERK, ATF6, and IRE1α (FIG. 10B). The plots made in FIG. 3B were then made by furthering reducing only the ICs that varied across perturbation (IC1-IC4 in FIG. 10B) or across the cell cycle (IC5-IC8 in FIG. 10B) to two dimensions using t-sne. (I.e., matrices with cells as rows and the given ICs as columns were constructed and those matrices were reduced to two dimensions with t-sne.) To make the plots in FIG. 3C, the population was subsampled to only look at cells treated with thapsigargin with or without depletion of PERK, and the DMSO treated control. The same methodology as above, though with 12 ICs instead of 16, was applied. The “G1 cell” IC described in the main text was bimodal within each subpopulation (see inset in right panel of FIG. 3C), but with varying distances between the two modes (note that the IC takes a substantially lower value in the +Tg population than in any of the others, FIG. 3C). Each population was split at the location of the mode. The cell cycle position histograms were made as described above. To make FIG. 3E, 25 genes that most positively influenced the IC and the 25 genes that most negatively influenced the IC (by sorting the mixing matrix column for that IC by coefficient value) were taken and clustered based on coexpression as described in the “Hierarchical clustering of genes” section. The meaning of each cluster was discerned by the pattern of up- and down-regulation observed within.

Branch Epistasis Analysis in FIG. 3F

Two populations: (1) consisting of cells treated with 100 nM thapsigargin in each of our 8 genetic backgrounds, along with DMSO-treated control cells, or (2) consisting of cells treated with 4 μg/mL tunicamycin in each of our 8 genetic backgrounds were created, along with DMSO-treated control cells. To identify informative differentially regulated genes, the random forest classifier method described in the “Identification of differentially expressed genes,” limiting the random forest to pick 100 genes for each of the two populations, was used. These two lists were combined into one list and any duplicate genes were discarded. Average profiles of expression of these genes for each of the nine conditions present in the two populations, as visualized in FIG. 3F, were created. The average epistatic phenotype of a gene can then be viewed as a 9-vector in either the thapsigargin- or tunicamycin-treated populations. Any genes where the correlation between these two conditions was less than 0.9 were discarded, as only factors that showed the same regulation in response to both conditions were of interest. The end result was the 104 genes presented in FIG. 3F. These were then clustered based on their co-expression pattern as described in the “Hierarchical clustering of genes” section, with the exception that Spearman correlation was used instead of Pearson correlation (to emphasize the large shifts in expression across the population). Rough meanings were ascribed to clusters based on the average pattern of gene expression across perturbations, but it should be emphasized that many targets show some degree of crossregulation. To assess this in an unbiased way, a matrix consisting of the average expression of the 104 assayed genes across the 17 unique conditions present in the experiment was constructed, and then reduced to four independent components using FastICA. Three of the components clearly corresponded to ATF6, IRE1α, and PERK perturbations, as they showed banded patterns in the reduced matrix matching the pattern of epistasis for those regulators seen in FIG. 3F (e.g. the PERK component was high in all PERK conditions, and low everywhere else). The fourth component was low in the DMSO and all tunicamycin-treated conditions, and high in the thapsigargin-treated condition, so it was discarded as representing the difference between the two perturbations. The panel at the bottom of FIG. 3F plots the mixing matrix coefficients for each gene in the indicated component, and thus determines how much that gene affects that component's value.

Genome-wide CRISPRi screen in FIG. 4 Analysis of the screen is described above along-side the experimental details above.

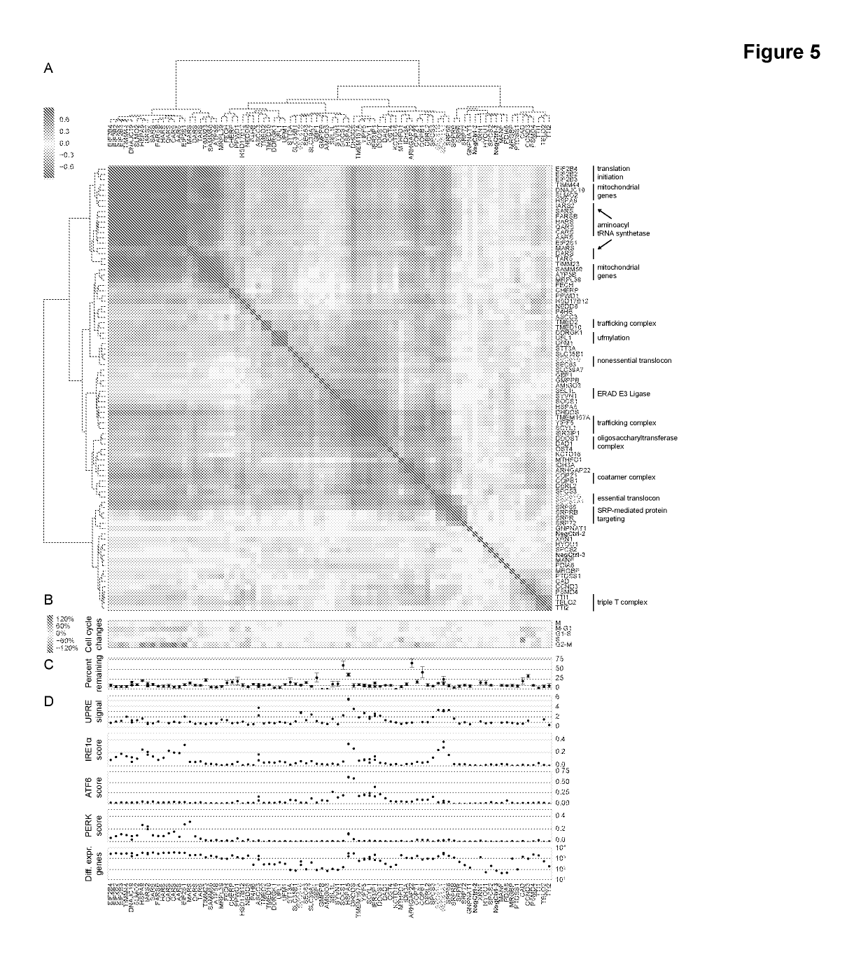

Clustering of Guides and Perturbations in FIG. 5

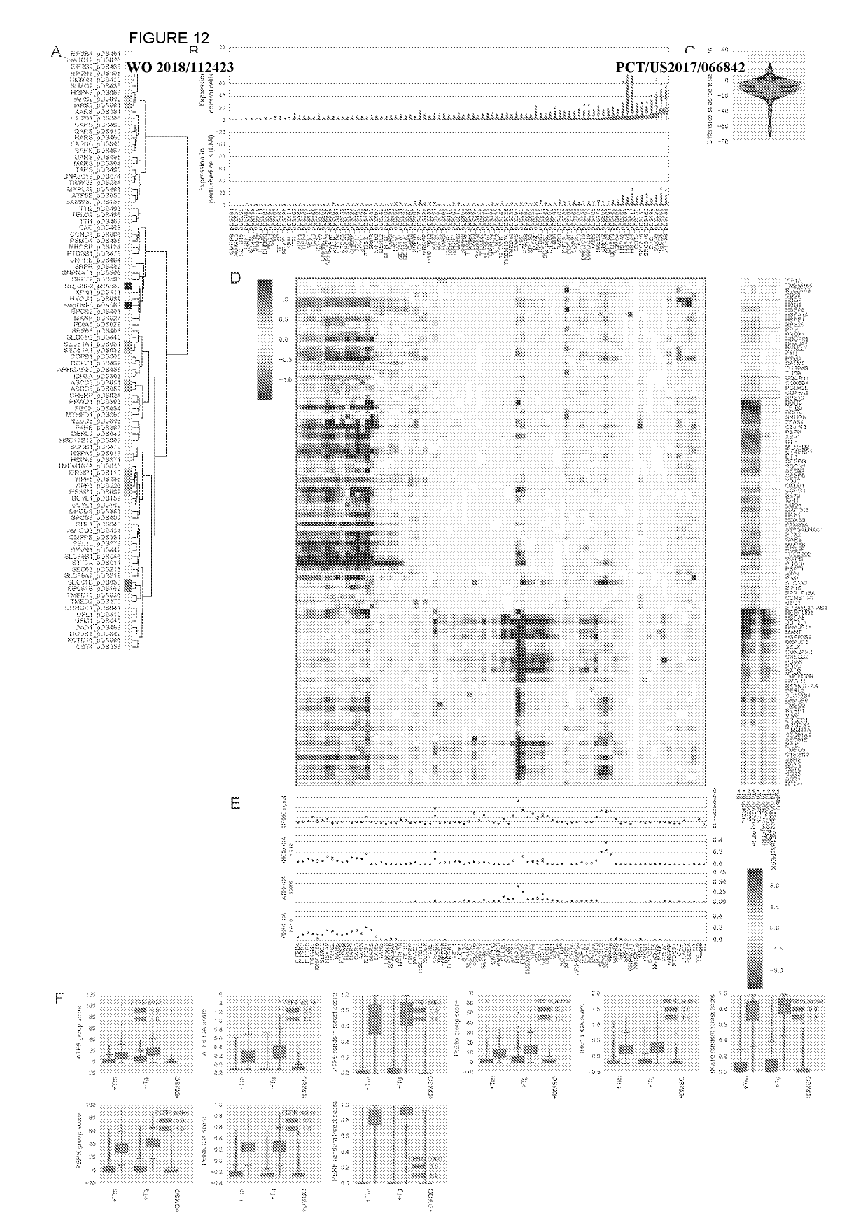

First, the large perturb-seq population was split into subpopulations based on guide identity and created average expression profiles (see “Average expression profiles” section) for each perturbations of all genes with mean representation >1 UMI per cell. The perturbation-perturbation correlation matrix was calculated between all average expression profiles and then clustered using the same methodology described in the “Hierarchical clustering of genes.” The ordering is seen in FIG. 12A. Because guides targeting the same gene behaved similarly in this analysis, in subsequent analyses the population was instead split into subpopulations based on guide target (thus merging subpopulations that had different guides that targeted the same gene). These profiles were clustered using the same criteria, the resulting dendrogram was optimally ordered and correlation matrix (as described in “Hierarchical clustering of genes”) to produce FIG. 5A.

Assessing Guide Homogeneity and Knockdown in FIG. 5

Most guide targets were too low abundance to interrogate directly at single-cell resolution. The shift in guide target expression induced by the guide was directly visualized, comparing the distribution of expression in control cells to cells perturbed for a given target (FIG. 12B). Mean knockdown per guide (FIG. 5C) was calculated and 95% confidence intervals were assigned to estimates via bootstrapping. The extent to which knockdown varied throughout the population based on phenotype was assessed. To do this, an unbiased means of assessing deviation in behavior from the control cells was necessary. A method called OneClassSVM (Scholkopf et al. 2001), which is a means of novelty detection, was leveraged. Given a set of training exemplars, a OneClassSVM learns an estimate of how those points are distributed (potentially in a high-dimensional space). When given new observations, the OneClassSVM then estimates how likely it is that those observations came from the same distribution as the training set, or if they are outliers (potentially novel). In this case, the OneClassSVM was trained using control cells, and thus scored the extent to which perturbed cells scored as outliers, or if they fell within the expected range of behavior for unperturbed cells. Specifically, for each guide target, the following algorithm was performed: 1. Form a population of all cells perturbed for that target, and an equal number of randomly sampled control cells. 2. Find all genes that are expressed at an average level of 0.5 UMI per cell or higher and that are differentially expressed between control and perturbed cells by the 79 Kolmogorov-Smirnov test (as described in “Identification of differentially expressed genes”) at P<0.01. 3. Form a reduced gene expression matrix consisting only of the differentially expressed genes. Create a low-dimensional picture of the dynamics within this population by reducing this matrix to 8 dimensions via PCA. 4. To form an estimate of “normal” behavior, train a OneClassSVM model to estimate the support of the control cells in this 8-dimensional space. The model was trained assuming a contamination rate with outliers of 5%. 5. Score each cell in the perturbed population using the OneClassSVM model to estimate the extent it deviates from control behavior. These scores generally assigned most or all of the perturbed cells outlier status, except in guides where very few genes were perturbed to begin with (bottom panel of FIG. 5D). Ordering the cells by score, each perturbed cell population was split into top third and bottom thirds (i.e. the most and least perturbed cells) and the difference in average knockdown in each of these populations (FIG. 12C) was assessed, with a difference of ˜8% on average. The number of differentially expressed genes measured above in the bottom panel of FIG. 5D was also reported.

Scoring Branch Activation in FIG. 5D

As outlined above, a data-driven strategy was adopted to score activation of each of the UPR branches using the epistasis experiment as training data. To do this, the label “ATF6 active”, “IRE1 active”, or “PERK active” was assigned to each cell in the epistasis experiment, based on whether a given branch was present (i.e. not depleted) and induced (tunicamycin or thapsigargin had been added). For example, cells treated with thapsigargin and depleted for IRE1α would have ATF6 and PERK active, but not IRE1α. These labels were converted to scores of 0, for inactive, and 1, for active, and then three random forest regressors were trained to predict activation of each branch. The training strategy was the same as outlined in the “Identification of differentially expressed 80 genes” section: each cell was regarded as a training data point, with every gene of mean >1 UMI initially regarded as a possible feature for predicting branch activation. Each regressor was constrained to use the top 25 genes for predicting branch activation, as no performance improvement was found when more genes were included. The genes isolated as most important by the three regressors for scoring activation of the three branches all appear in the epistasis analysis in FIG. 3F. To assess performance, this approach was compared to scoring based on two other strategies: 1. Gene list approach: A list of hand-picked branch-specific genes were chosen from FIG. 3F, and a score was defined as the sum of the normalized expression of those genes. 2. ICA approach: To allow for more complicated logic than simple sums, we used the ICA decomposition seen in FIG. 3F and computed the value of each IC for every cell in the population. With each scoring system, the median of the DMSO-treated control cells' were substracted and all cells with negative scores were thresholded to zero. Then, overlap of score distributions between cells expected to have a given branch active or inactive was assessed. Both the gene list approach and the ICA approach performed worse than the random forests in this analysis (FIG. 12F). The branch scores seen in FIG. 5D are the result of applying the random forest regressor scoring system to each cell in the perturb-seq experiment, and then averaging the results within cells knocked down for the same gene. The average scores assigned by the ICA method agree well (cf. FIG. 5D and FIG. 12E).

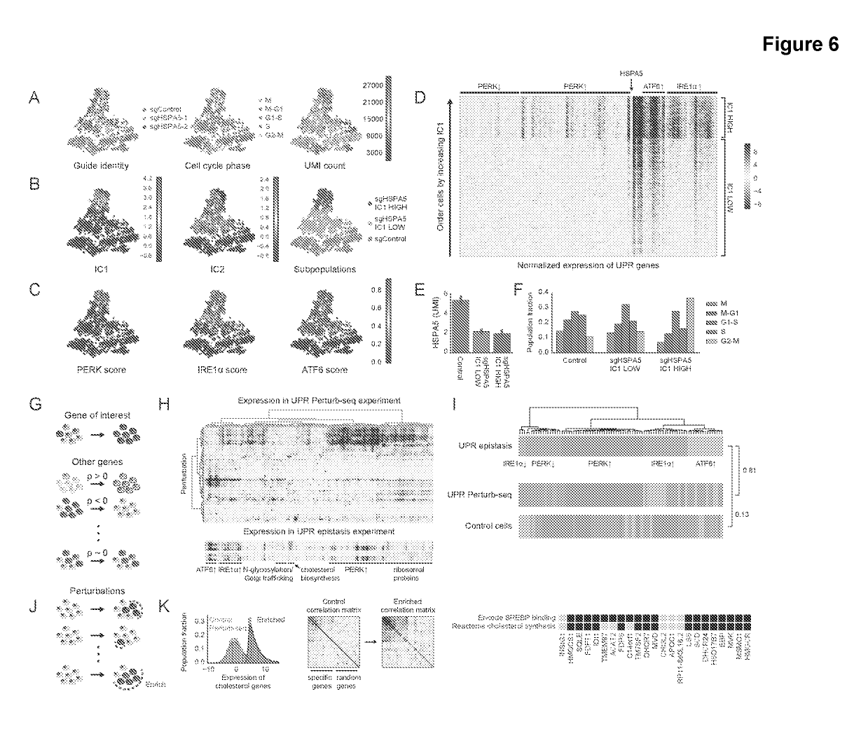

Single-Cell Analysis in FIG. 6

A population of cells containing either our two guides targeting HSPA5, or the NegCtrl2 guide was formed. All genes that had mean abundance >0.5 UMI per cell and that were differentially expressed between the two populations by Kolmogorov-Smirnov test (P<0.01) were found, resulting in ˜2,100 genes. A reduced gene expression matrix consisting only of these genes was formed and low rank ICA was applied to reduce the population's dynamics therein to 12 ICs. The t-sne plots were made by reducing the low rank matrix to 16 components using PCA and then applying t-sne (see “t-sne visualization” section). Branch activation scores in FIG. 6C were assigned as described above in the “Scoring branch activation in FIG. 5D” section. Two ICs varied substantially in average value between the control and perturbed cells (FIG. 6B). The first, IC1, had a two-phase distribution in which all control cells and the majority of HSPA5-perturbed cells fell in the large lower peak, and a subpopulation of HSPA5-perturbed cells fell into a long tail of higher values (FIG. 6B). The HSPA5 IC1 HIGH cells were defined to be the ones that fell within this tail (FIG. 6B). FIG. 6D shows the normalized expression of genes found in our epistasis analysis (FIG. 3F) as columns, and the HSPA5-perturbed cells as rows, ordered by increasing IC1. FIG. 6E was created simply by averaging the expression of HSPA5 within the subpopulations defined in FIG. 6B. FIG. 6F was created using the cell cycle positions called in the “Cell cycle position” section.

Gene Clustering Analysis in FIG. 6H

An unbiased approach to find programs of gene expression induced in the perturb-seq experiment was needed. To do this, the population was separated into control cells (containing our two control guides) and perturbed cells (containing any guide). Average expression profiles (see “Average expression profiles” section) of each were constructed, and then the analysis was restricted to genes of mean expression >0.5 UMI per cell on average in the perturbed population, and whose normalized expression was >0.5. (Control cells by definition have mean normalized expression 0 for all genes, see “Expression normalization” section.). Then, a random forest classifier approach was used to select 200 of these induced genes that varied informatively across all of the perturbations in the perturb-seq experiment (see “Identification of differentially expressed genes” section). The genes were then clustered based on their co-expression throughout the population, with the dendrogram leaves optimally reordered (see “Hierarchical clustering of genes” section). The assumption was that many of these “induced genes” were involved in the unfolded protein response. UPR dependence was evaluated by examining the expression pattern of the induced genes within thapsigargin- and tunicamycin-treated cells (FIG. 13B). Identities were also assigned to some other clusters based on clear functional connections (as seen in FIG. 6H).

Comparison of Clustering of UPR Genes in FIG. 6I and FIG. 13A

As many UPR genes fell out of the previous analysis, the ability to go the opposite direction, and cluster known interactions, was evaluated. Thus, the list of UPR-regulated genes found in FIG. 3F was reexamined. The perturb-seq population was separated into control cells (containing our two control guides) and perturbed cells (containing any guide). Average expression profiles (see “Average expression profiles” section) of each were constructed, and then the analysis was restricted to the UPR-regulated genes that showed the same pattern of induction or repression in the perturbed cell population as they did in the cells treated with thapsigargin in the epistasis experiment that had all branches of the UPR intact. Hierarchical clustering of these genes (see “Hierarchical clustering of genes” section) was performed using co-expression information from either (1) all cells in the epistasis experiment, (2) all cells in the perturb-seq, and (3) only control cells in the perturb-seq experiment. The similarity among clusterings was assessed using the cophenetic correlation coefficient, i.e. the correlation coefficient between dendrogram distances taken over all possible pairs of genes. Closeness in cophenetic correlation thus implies that the dendrograms tend to place the same genes close to each other. The figure is meant only as a visual aid, as the cophenetic correlation relies on information beyond the linear order. The genes were roughly grouped based on their epistasis pattern in the epistasis experiment (as in FIG. 3F), and then color was preserved as they were shuffled by the other two clusterings.

Enrichment of Cholesterol Genes in FIG. 6K

The unbiased analysis in FIG. 6H contained a cluster of genes involved in cholesterol biosynthesis: ACAT2, FDPS, FADS1, INSIG1, TMEM97. A “cholesterol score” was made by summing the normalized expression of this group of genes in each cell, and then a subpopulation containing (1) cells with cholesterol scores at or above the 95% of the control cell population and (2) control cells was created. This gave 9,000 cells. Within this subpopulation, the cholesterol score was correlated with the normalized expression of all genes with mean >0.25 UMI per cell. Then, all genes that had a correlation of 0.15 or higher with the cholesterol score were selected for further analysis. Genes were clustered by co-expression within the population (see “Hierarchical clustering of genes” section), and then a group of 23 genes that clustered together with the original five and that appeared as a distinct block on the diagonal of the gene-gene correlation matrix were selected. To demonstrate the improvement in correlation obtained by this “fishing” approach, correlation matrices composed of these 23 genes and 23 random genes of similar average abundance between our enriched population, and control cells (seen in FIG. 6K) were compared. Finally, Enrichr (Kuleshov et al. 2016) was used to obtain Reactome annotations and Encode SREBP binding state. Note that some of the genes that don't have annotations nevertheless are almost certainly cholesterol-related, such as the lncRNA RP11-660L16 which is directly next to DHC7R.

Enrichment of Heat Shock Genes in FIG. 6L

An identical approach to the above was followed, except starting with the genes HSPA1A and HSPA1B. In this case all of the genes that had correlations of 0.15 or greater are presented. Enrichr was used to find the top 3 most enriched transcription factor binding sites among the set of genes, as presented in FIG. 6L.

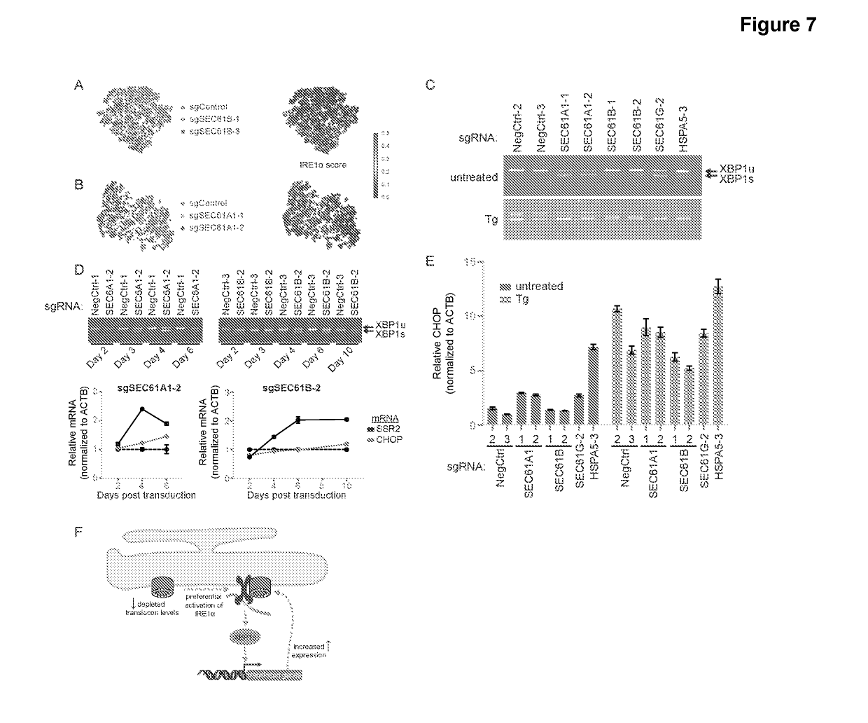

Single-Cell Analysis in FIG. 7

Populations of cells containing guides targeting either SEC61A1 or SEC61B were formed, along with cells containing the NegCtrl2 guide. All that had mean abundance >0.5 UMI per cell and that were differentially expressed between the two 84 populations by Kolmogorov-Smirnov test setting a threshold of D>0.15 for SEC61A1, and D>0.1 for SEC61B, which is a weaker perturbation (see “Identification of differentially expressed genes” section), were found. The different thresholds were chosen largely for esthetic reasons: lowering the threshold with SEC61A1, which is a strong perturbation, resulted in the inclusion of a number of cell cycle genes that caused the control population to fragment into subpopulations by cell cycle phase, which was distracting. In each case, a reduced gene expression matrix consisting only of differentially expressed genes was formed. Then, robust PCA (see “Low rank ICA” section) was applied to these matrices, and the dynamics were visualized using t-sne plots generated using the first 16 principal components (see “t-sne visualization” section). Branch activation scores in FIGS. 7A, B, 14A were assigned as described above in the “Scoring branch activation in FIG. 5D” section.

Data and Software Availability Custom Python scripts for analysis of genome-scale CRISPRi screens is available at https://github.com/mhorlbeck/ScreenProcessing.

Sequencing Frozen samples of between 250×10⁶-2×10⁹ cells collected at T0 and endpoint were processed to isolate genomic DNA by standard methods. The DNA encoding the sgRNA was enriched from bulk genomic DNA by digestion of genomic DNA using Sbf1 or Mfe1 restriction sites encoded in the lentiviral vector followed by gel electrophoresis and gel extraction as previously described (Gilbert & Horlbeck Cell 2014, Horlbeck elife 2016). The sgRNA-encoding regions were amplified from enriched genomic DNA and sequenced on an Illumina HiSeq-2500 or 4000 using custom primers described in FIG. 15. Sequencing reads were aligned to the expected library sequences using Bowtie (v1.0.0, Langmead et al., 2009) and read counts were processed using custom Python scripts (available at https://github.com/mhorlbeck/ScreenProcessing) based on previously established sgRNA screen analysis pipelines (Gilbert & Horlbeck cell 2014 and Horlbeck elife 2016). sgRNAs represented with fewer than 10 or 50 sequencing reads in both T0 and Endpoint were excluded from analysis. sgRNA growth phenotypes (γ) were calculated by normalizing sgRNA loge enrichment from T0 to endpoint samples and normalizing by the number of cell doublings in this time period.

### Results

A Robust Strategy for Pooled Profiling of Perturbed Cells by Single-Cell RNA-Seq

Massively parallel droplet-based approaches for single-cell gene expression profiling incorporate two indexing strategies that allow pooled RNA-seq data to be deconvolved into single-cell transcriptomes (Klein et al., 2015; Macosko et al., 2015; Zheng et al., 2016) (FIG. 1A). Briefly, mRNA molecules from single cells are paired in-droplet with two types of index, a cell barcode (CBC) and a unique molecule identifier (UMI). These indices are affixed to cDNA molecules during reverse transcription and, after pooled RNA-seq library preparation, are read out with mRNA identity by sequencing. The CBC links all sequencing reads from a given cell, and the UMI enables molecular counting of captured mRNA molecules by correcting for PCR amplifications. On these platforms, such indexing relies on oligo-dT priming prior to cDNA synthesis and, therefore, captures only polyadenylated RNA transcripts. To enable the recording of other types of information, a platform to genetically encode a third type of index on a synthetic polyadenylated transcript (FIGS. 1A, 1B) was built. This index, which is termed a “guide barcode” (GBC), can mark specific cell perturbations (e.g., the identity of a Cas9-targeting single guide RNA, sgRNA), and thus allow complex pools of cells to be interrogated in parallel on existing droplet-based platforms.

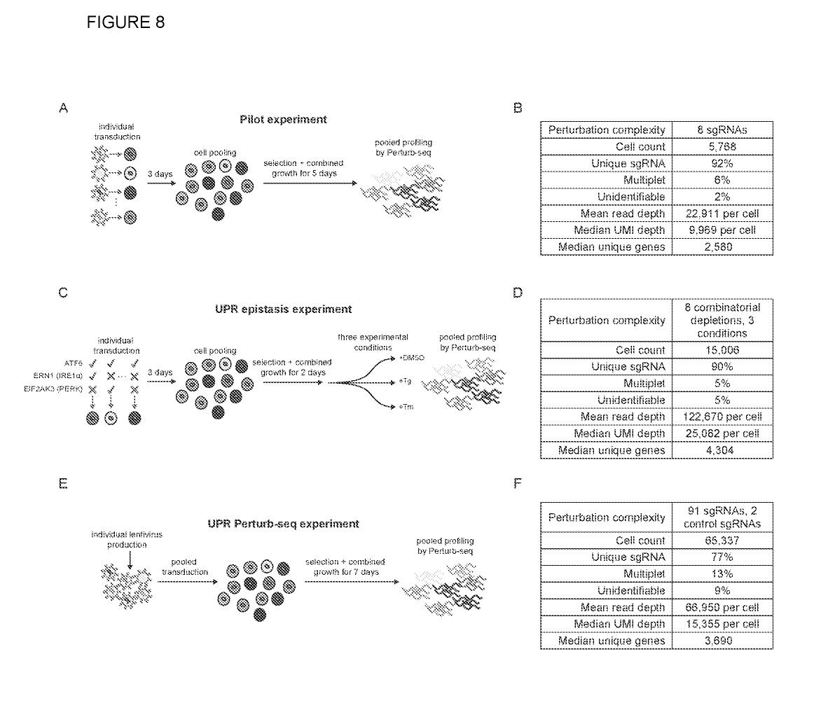

To deliver and capture GBCs, the “Perturb-seq vector,” a third generation lentiviral vector that contains two notable features: an RNA polymerase II-driven “GBC expression cassette” and an RNA polymerase III-driven “sgRNA expression cassette” (FIG. 1B) was designed. The GBC expression cassette carries a 3′ GBC sequence and terminates with a strong polyadenylation signal (BGH pA). Close proximity of the GBC and the BGH pA within this cassette favors faithful transmission of GBC sequences into single-cell RNA-seq libraries, which typically capture only the 3′ ends of transcripts. To prevent the internal BGH pA from disrupting transcription of the lentiviral genome, and therefore transduction competency, the entire expression region was engineered in reverse orientation with respect to the genomic promoter. Finally, to ensure robust GBC capture, a PCR protocol was developed to specifically enrich GBC-containing cDNAs, or “guide-mapping amplicons,” out of single-cell RNA-seq libraries (FIGS. 1A, 1B). In a pilot experiment, single-cell RNA-sequencing was performed on a pool of individually transduced chronic myeloid leukemia cells (K562) carrying 8 distinct GBCs, analyzing 5,768 cells total (FIGS. 8A, 8B). For the vast majority of cells, sequencing of guide-mapping amplicons uniquely identified a single dominant GBC with strong enrichment over any competing GBC (FIG. 1C). Moreover, a median of 45 independent observations per cell of these dominant guide-mapping amplicons (marked by UMIs) were observed, allowing us to uniquely infer a single GBC for 92.2% of cells (FIGS. 1D, 1E, 8B). Similar overall mapping rates were observed in all subsequent experiments (FIG. 8). Importantly, our high confidence in GBC calling allowed us to discard information from droplets that fortuitously received more than one cell (FIG. 1C).

By including an sgRNA expression cassette in the Perturb-seq vector, we tailored our indexing system to the study of CRISPR-based phenotypes. We confirmed that sgRNA expression from the Perturb-seq vector was capable of generating robust and homogeneous CRISPRi-mediated gene repression, as activity against genomically integrated GFP (using sgGFP, an sgRNA programmed with the previously validated EGFP-NT2 protospacer (Table 2) (Gilbert et al., 2013)) was robust and comparable to that from a previously validated sgRNA expression construct (95.4% and 96.2% reduction of GFP fluorescence, respectively) (FIG. 1F, Methods) (Gilbert et al., 2014).

A Strategy for Multiplexed sgRNA Delivery to Allow Simultaneous Genetic Perturbations

To systematically delineate IRE1α-, PERK-, and ATF6-controlled transcriptional programs and to expand Perturb-seq to the analysis of higher-order genetic interactions, we sought to design a vector that could mediate robust and homogeneous perturbation of gene combinations in individual cells. Previous efforts to simultaneously express different sgRNAs (for targeting Cas9) have had limited success achieving uniform genetic perturbations across multiple targets (Kabadi et al., 2014; Nissim et al., 2014). In engineering our vector, we first incorporated three tandem sgRNA expression cassettes (composed of an RNA polymerase III promoter, sgRNA protospacer, and sgRNA constant region) into our Perturb-seq vector (FIG. 2A). To minimize intramolecular recombination at repetitive nucleotide sequences during lentiviral transduction (Sack et al., 2016; Smyth et al., 2012), we used three different promoters in this initial three-guide vector (Methods). Test vectors expressing sgGFP from one of the promoters (and negative control sgRNAs from the others) partitioned GFP+ K562 cells with dCas9-KRAB into two subpopulations with either strong GFP depletion (>90%) or no detectable depletion (FIGS. 9A, 9B). Such incomplete activity could result from a remaining propensity for recombination between the 93-nt sgRNA constant regions or limiting dCas9-KRAB levels when expressing multiple sgRNAs. To test the latter possibility, we generated GFP+ K562 cells with 10-fold higher dCas9-KRAB levels (cMJ006 cells) (FIG. 9C). However, GFP depletion remained bimodal when expressing sgGFP from one of our initial three-guide vectors in these cells (FIG. 2B).

To solve this problem, we next engineered two modified sgRNA constant regions (cr2 and cr3) that share at most 20 bases of continuous sequence homology with each other and the original constant region (cr1) (FIGS. 2C, 9D, 9, Methods). These constant regions were functional in bacteria and, when paired with the EGFP-NT2 protospacer and expressed from modified mouse (mU6) and human U6 (hU6) promoters, respectively, mediated GFP depletion in K562 cells that was indistinguishable from that of the Perturb-seq vector or sgGFP expressed from a modified bovine U6 (bU6) promoter (FIGS. 2C, 2D, 9E, Methods). We then designed our final three-guide Perturb-seq vector with the following sgRNA expression cassettes: the bU6 promoter paired with cr1, mU6 with cr2, and hU6 with cr3 (FIGS. 2A, 9F). Vectors expressing sgRNAs programmed with EGFP-NT2 from any of the three cassettes in this final design mediated near-uniform and strong depletion of GFP (96-97%), nearly identical to that mediated by the Perturb-seq vector (FIG. 2E, Methods). Thus, our final three-guide vector can be faithfully delivered by lentiviral transduction and mediates robust knockdown of targeted genes.

Systematic Delineation of the Three Branches of the UPR Using Perturb-Seq

With these tools in hand, we applied Perturb-seq to explore the branches of the mammalian UPR (FIGS. 3A, 8C). Using our three-guide Perturb-seq vector, we introduced sgRNAs targeting each UPR sensor gene in all possible single, double, and triple combinations into K562 cells with dCas9-KRAB (Methods). Transduced cells were then pooled, sorted for vector delivery, and, after 5 days of total growth, treated with pharmacological inducers of the UPR: thapsigargin, an ER calcium pump inhibitor, or tunicamycin, an inhibitor of N-linked glycosylation. Control cells were treated with DMSO. We sequenced transcriptomes of ˜15,000 cells (FIG. 8D). Critically, across all conditions, we observed >80% depletion of targeted genes (FIG. 2F). Throughout, we refer to this experiment as our “UPR epistasis experiment.”

We then devised an analytical approach for finding robust features within the data (FIGS. 3B, 10A). Single-cell RNA-seq data are rich, but intrinsically noisy and of very high-dimension. However, many genes share common regulation, arguing that cellular behavior is intrinsically low-dimensional (Heimberg et al., 2016). This motivates the use of unsupervised dimensionality reduction methods, describing cellular behavior in terms of tens of components rather than thousands of genes.

To uncover this latent low-dimensional behavior in a way that is robust to noise, we developed low rank independent component analysis (LRICA, Methods). We applied recent advances in sparse matrix theory (Candès et al., 2011; Lin et al., 2010) to decompose the observed gene expression matrix (X) into a low-rank matrix (L), representing the low-dimensional dynamics of the population, and a sparse matrix (S), capturing noise and effects that are highly variable between cells:

X=L+S

We then identify informative trends in the low-dimensional dynamics by applying independent component analysis (ICA, Methods) to the matrix L. The components aid interpretation in two ways: components that are bimodal define subpopulations and, by asking which genes influence a component, we can identify those driving a behavior.

We applied LRICA to our thapsigargin-treated cells. Four components varied across the different perturbations, including three that tracked the presence of PERK, IRE1α, and ATF6 (Methods, FIG. 10B). When projected to two dimensions using t-distributed stochastic neighbor embedding (t-sne) (Van Der Maaten, 2014), cells bearing a particular perturbation all grouped together, further validating our triple depletion strategy, and biologically reasonable groups of perturbations clustered (FIG. 3B). The same analysis applied to the four components that varied across the cell cycle arranged the cells in a circular pattern ordered by cell cycle phase (FIGS. 3B, 10B). Thus, LRICA identified and decomposed the two largest effects causing variation in the population in an unbiased way and computationally decoupled them from each other.

We did observe an interaction between the two effects, apparent in a “bulge” in FIG. 3B (dashed black line in bottom right panel). Closer analysis of three conditions showed this interaction was caused by PERK-dependent cell cycle arrest in G1 caused by thapsigargin treatment (FIGS. 3C, 3D) (Hamanaka et al., 2005). One component (right panel of FIG. 3C) was bimodal among the cells bearing each perturbation. Defining the cells with that component low as “G1 cells” (cf. middle and right panels of FIG. 3C), we looked at the top fifty genes influencing the component (FIG. 3E) and noted epistatic interactions between PERK-dependent UPR activation and progression through G1. For some genes the two programs cancel each other out, while for others they act synergistically, as in the thapsigargin-induced expression of MYC (Liang et al., 2006), which our data show is most strongly associated with the G1-arrested subpopulation (FIG. 3E).

We next turned to delineating the three transcriptional programs of the UPR. We identified a set of genes robustly induced by both thapsigargin and tunicamycin treatment and hierarchically clustered them based on their co-expression (Methods). When synthetic bulk RNA-seq profiles (made by averaging all cells containing the same GBC for a given treatment) were ordered according to our clustering, patterns of regulatory control were apparent (FIG. 3F). To estimate regulatory overlap, we used ICA to decompose the changes across bulk responses (bottom of FIG. 3F, Methods). PERK/ATF4 had the largest regulon in our experiment, with many targets uniquely under its control. ATF6 and IRE1α showed more overlap, consistent with a more common transcriptional regulatory mechanism (Yamamoto et al., 2007). Of the two, IRE1α had more specific targets, notably components of the translocon and translocon auxiliary components (consistent with previous reports (Shoulders et al., 2013)), but ATF6 had stronger activating effects on common targets (FIG. 3F). Many genes showed some sensitivity to all branches, particularly a group of very high abundance stress response genes (HSPA5, HERPUD1, SDF2L1). Our experiment thus defined and decoupled the three overlapping branches of the mammalian UPR, both at the bulk level and within single cells.

Genome-Scale CRISPRi Screens Identify Genetic Perturbations that Induce the UPR

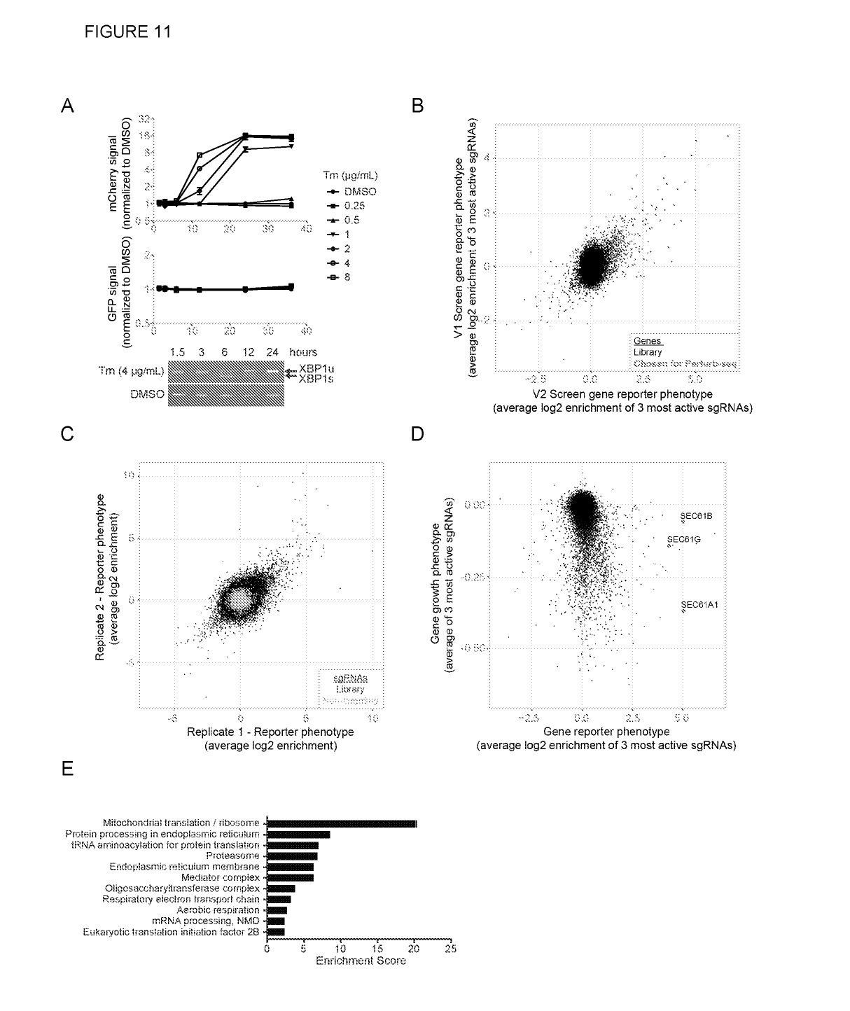

We next employed a two-tiered approach to systematically evaluate how UPR transcriptional programs respond to various perturbations. First, we performed two genome-scale CRISPRi screens that identified genes important in maintaining ER homeostasis. For this, we built a K562 cell line (cBA011) that stably carries dCas9-KRAB, an mCherry reporter of IRE1α activation, and (to control for general effects on gene expression) a constitutively expressed GFP reporter driven by the EF1a promoter (FIG. 4A). Importantly, when treated with tunicamycin, these cells demonstrated XBP1-dependent mCherry induction (maximally 16-fold), which occurred subsequent to endogenous XBP1 splicing (FIGS. 4B, 11A). As expected, we observed no similar induction of GFP.

Using our reporter cell line, we separately screened two genome-scale CRISPRi libraries, our first generation library (CRISPRi-v1), which targets 15,977 genes (20,899 transcriptional start sites, TSSs) with 10 sgRNAs per TSS, and our recently described second-generation library (CRISPRi-v2), which targets 18,905 genes (20,526 TSSs) with 5 sgRNAs per gene (FIGS. 4C, 4D, 11B, 11C) (Gilbert et al., 2014; Horlbeck et al., 2016). Briefly, reporter cells (cBA011) transduced with each library were grown for 8 days and then separated into bins according to their ratiometric reporter signal (mCherry/GFP) by FACS. Cells in the top and bottom thirds of this reporter distribution were collected and processed to measure the frequencies of sgRNAs contained within each, from which we calculated sgRNA and gene-level reporter signal phenotypes. Our CRISPRi-v2 screen identified 397 hit genes with high mCherry/GFP, indicative of UPR activation (FIG. 4D). Importantly, phenotypes were reproducible between replicates and minimal correlation was observed between hit phenotypes and previously calculated gene growth phenotypes (Spearman R=-0.2) (FIGS. 11C, 11D). Of the 141 hits from the CRISPRi-v1 screen, 103 reproduced from screening the CRISPRi-v2 library (Fisher's Exact p-value=8.97e-138) (FIG. 11B).

Among hits from the CRISPRi-v2 screen are well-characterized regulators of protein folding in the ER, most notably HSPA5, which encodes the major ER Hsp70 chaperone BiP (FIG. 4E). Consistent with results from a similar screen in yeast (Jonikas et al., 2009), our hits featured genes involved in N-linked glycosylation, including components of the oligosaccharyltransferase (OST) and the dolichol-linked oligosaccharide biosynthesis pathway, ER-associated degradation (ERAD), and protein trafficking. Additionally, genes involved in SRP-mediated protein targeting to the ER were enriched among hits (Fisher's Exact p-value=2.65e-09). Three out four of the translocon-associated protein complex (TRAP) scored; and strikingly, among the 7 hits with the strongest phenotypes were all three genes that encode the ER protein-translocation channel or translocon (SEC61A1, SEC61B, SEC61G) (FIGS. 4D, 4E, 11D). The phenotypes of SRP targeting factors and the translocon were surprising because recent reports have shown that SRP-mediated recruitment of unspliced XBP1 (XBP1u) to the ER and IRE1α binding to the translocon are required for maximal XBP1 splicing in response to exogenous stress (Kanda et al., 2016; Plumb et al., 2015). Satisfyingly, targeting of both ERN1 (IRE1α) and XBP1 decreased reporter signal in the screen (FIG. 4D).

Genes with biological functions not known to be directly related to ER function also scored among hits, some of which are distinct from functional classes seen in the analogous systematic yeast studies (Jonikas et al., 2009). Sets of genes that control general translation, transcription, and, perhaps most intriguingly, mitochondrial function were enriched among hits (FIGS. 4E, 11E). While intriguing, these phenotypes alone give us little power to infer mechanisms by which gene repression disrupted ER homeostasis. Additionally, while disruption of these gene functions may impair ER function, it is also possible that such hits represent UPR-independent effects on our reporter system. Individual testing of 257 sgRNAs targeting 152 select hit genes confirmed that a majority induced UPRE reporter signaling; however, some of these sgRNAs, notably ones targeting the mediator transcriptional complex, also reduced GFP levels compared to controls (FIG. 4F).

Perturb-Seq of UPR-Inducing CRISPRi Sub-Library Reveals Functional Relationships

Next, to characterize the role of these different gene classes, we applied Perturb-seq to a small CRISPRi library of 91 sgRNAs targeting 83 genes, including many of our strongest hits, and 2 negative controls (FIG. 11B). To test platform scalability, sgRNAs were delivered via pooled transduction using a mixture of separately prepared lentiviruses, and we collected 65,000 transcriptomes in one large pooled experiment (FIGS. 8E, 8F, Methods). Throughout, we refer to this experiment as our “UPR Perturb-seq experiment.” All expected sgRNAs (i.e. GBCs) were detected, with expected and even representation (457±108 cells per sgRNA, mean±standard deviation).

To explore these data, we first constructed synthetic bulk expression profiles by averaging normalized expression across cells containing each sgRNA (i.e. GBC). Hierarchical clustering of these profiles revealed that sgRNAs targeting the same gene clustered together (FIG. 12A). Knockdown was robust, with median 90% depletion of the guide target and similar levels of depletion between sgRNAs with the same target (FIG. 5C). Target depletion occurred as a shift in the expression distribution, rather than a bifurcation into perturbed and unperturbed subpopulations (FIG. 12B). Indeed, when we computationally split each sgRNA-perturbed subpopulation into most- and least-perturbed (Methods), we observed a median difference in knockdown of 8% (FIG. 12C). These findings confirm the ability of CRISPRi to produce uniform knockdown as well as the ability of the barcoding scheme to accurately assign sgRNAs to the appropriate cells. Given the similarity in phenotypes between sgRNAs targeting the same gene, in subsequent analyses we grouped cells by sgRNA target rather than by sgRNA.

The bulk profiles are rich phenotypic fingerprints that identify how different perturbations are related. Hierarchical clustering of profiles revealed gene clusters (boxes on the diagonal in FIG. 5A) consistent with known functional and physical interactions, including those composed of genes involved in SRP-mediated protein targeting (SRP68/SRP72 and SRPRB/SRPR), UFMylation (UFL1/UFM1/DDRGK1), the ubiquitylation reactions of ERAD (SYVN1/SEL1L) and protein trafficking (TMED2/TMED10) (FIG. 5A). Perturb-seq can also yield insights at the single-cell level. For example, decomposing the populations by cell cycle position revealed that perturbation of many aminoacyl tRNA synthetases elicited an accumulation of cells in G2 (FIG. 5B).

We next sought to analyze how individual hits effect activation of the different branches of the UPR. We adopted a data-driven strategy and trained random forest regressors to score branch activation using the cells in our UPR epistasis experiment, in which the branches are definitively separated, as training data (Methods). This scoring method performed well and had better accuracy than other metrics (Methods, FIG. 12F). Branch activation scores (FIG. 5D) showed that hits from the screen activated all three UPR branches with clear correlations in activation among functionally related groups of genes. Intriguingly, different groups elicited differential activation of the three branches. For example, repression of HSPA5, which encodes the major ER chaperone BiP, robustly activated all three branches. Repression of the aminoacyl tRNA synthetases activated both IRE1α and ATF4 transcriptional programs. Finally, repression of all three subunits of the translocon (SEC61A1/SEC61G/SEC61B) appeared to selectively activate only the IRE1α branch. Comparison with alternate scoring methods and expression of UPR-controlled genes showed good agreement with these calls (FIGS. 12D, 12E). Thus our data reveal how different genetic perturbations can selectively activate the different branches of the UPR.

Single-Cell Analysis Uncovers a Bifurcated Response in HSPA5-Perturbed Cells

The above observation raises an immediate question: can the UPR branches also operate independently at the single-cell level? To explore this issue, we examined cells depleted of BiP, where all three branches of the UPR are active.

When compared to unperturbed cells, cells transduced with HSPA5-targeting sgRNAs were distinguishable as a distinct population (FIG. 6A), and had markedly different patterns of gene expression (˜2,100 genes differentially expressed at P<0.01, Methods). Using LRICA, we decomposed these differences into 16 independent components. Two of these (IC1 and IC2) varied substantially between control and HSPA5-perturbed cells (FIG. 6B), and were strongly influenced by UPR-responsive genes. Comparing these hypothesis-free results to the branch activation scores (FIG. 6C) showed that our analysis pipeline had independently discovered a subpopulation structure with differential activation of the UPR branches within HSPA5-perturbed cells. Indeed, when we ordered the cells by the value of IC1 and examined the expression of UPR-induced genes (as defined in FIG. 3F), the trends defining these subpopulations were apparent (FIG. 6D).

Of particular note was the switch-like induction of the PERK/ATF4 regulon, revealing that these cells represented a discrete subpopulation. These differences did not reflect levels of BiP depletion, as the subpopulations with IC1 low and high (FIGS. 6B, 6D) had equal expression of HSPA5 (FIG. 6E). However, the PERK/ATF4-induced subpopulation did have an altered cell cycle, with many cells accumulating in G2 (FIG. 6F). These results reveal that the UPR can be executed in markedly different ways within an apparently homogeneous population.

Gene-Gene Covariance Analysis of Perturb-Seq Data Reveals Transcriptional Regulons

FIG. 6D underscores a key point: correlated up- or down-regulation of genes can be a signature of shared regulation. As perturbations elicit coordinated changes, we reasoned that Perturb-seq could help identify related genes (FIG. 6G) (Klein et al., 2015).

For example, we identified 200 genes broadly induced in our UPR Perturb-seq experiment (Methods). When clustered based on co-expression, functional groups appeared, including all three UPR branches (FIGS. 6H, 13A). Moreover, when we clustered UPR-induced genes (from FIG. 3F) using co-expression in either the UPR epistasis experiment or the UPR Perturb-seq experiment, we obtained similar results (cophenetic correlation 0.81, compared to 0.13 when control cells were used) (FIGS. 6I, 13B, Methods), suggesting that the organization of the UPR is similar between commonly used strong chemical perturbants and the more varied genetic perturbations used here.

We finally investigated a “fishing” strategy to further enhance weak correlations (FIGS. 6J, 6K, Methods). Our initial analysis (FIG. 6H) identified 5 cholesterol biosynthesis genes with correlated expression. When we confined our gene clustering analysis to the ˜9,000 cells most perturbed for these genes, we saw strengthened correlations and the emergence of a larger cluster of cholesterol biosynthesis genes grouping together (FIG. 6K). Though these demonstrations are not proof, they suggest that correlation information from Perturb-seq may enable automated functional clustering of genes of unknown function.

A Homeostatic Feedback Loop Between the Translocon and the IRE1α Branch of the UPR

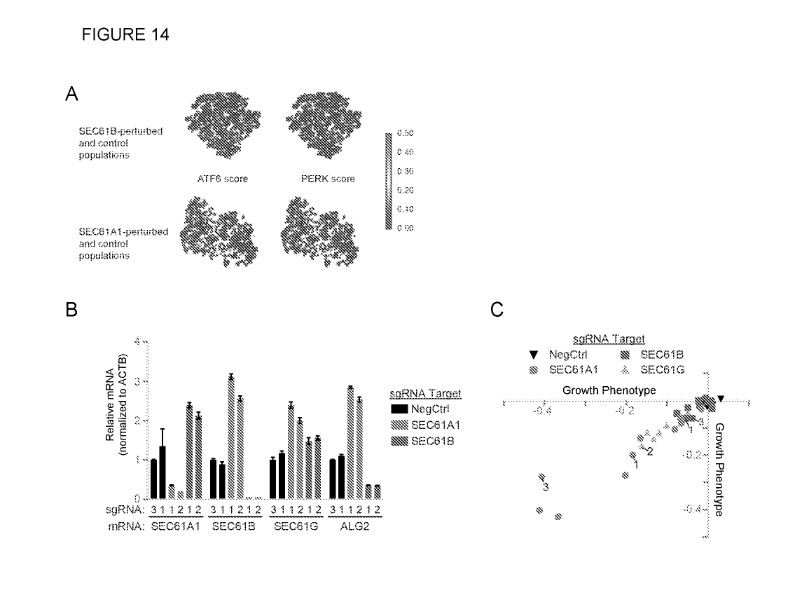

Among genes targeted in the UPR Perturb-seq experiment, SEC61A1, SEC61G, and SEC61B were perhaps the most intriguing outliers. Repression of each of these displayed a marked preference for activation of the IRE1α branch with little or no activation of the other branches (FIGS. 5D, 7A, 7B, 12E, 14A). To confirm that our single-cell data were accurately calling IRE1α activation, we directly probed for XBP1 splicing. Targeting all three translocon subunits induced XBP1 splicing at levels consistent with the single-cell data and to a degree at or above that provoked by targeting HSPA5, whose depletion induces all three branches of the UPR (FIGS. 5D, 6C, 6D, 7C, Methods). Additionally, repression of SEC61A1 and SEC61B led to sustained XBP1 splicing and upregulation of SSR2, a translocon auxiliary protein and strongly selective target of IRE1α (FIGS. 3F, 7D, 14B, Methods). These results were in contrast to transient XBP1 splicing caused by chemical stress, which diminished on the scale of hours, consistent with previous reports (FIG. 11A) (Lin et al., 2007). We note that SEC61B appears to share a co-regulated promoter region with ALG2, a gene that functions in N-linked glycosylation, and as such, we cannot formally separate the effects of repressing these genes (FIG. 14B). Nonetheless, the consistent phenotypes from targeting SEC61A1, SEC61B, and SEC61G suggest that translocon depletion elicits selective activation of the IRE1α branch.

To further investigate branch selectivity, we evaluated induction of CHOP, also called DDIT3 and a selective target of PERK/ATF4, after SEC61A1 and SEC61B repression (FIGS. 3F, 7D, 7E, Methods). Repression of SEC61B showed little to no CHOP induction. We observed a limited increase in CHOP expression in response to SEC61A1 repression but at lower levels than in cells transduced with an HSPA5-targeting sgRNA, and we reason that this could reflect general toxicity. Indeed, SEC61A1 is an essential gene, perturbation of which, unlike SEC61B, caused strong growth phenotypes in both CRISPRi and CRISPR cutting cell viability screens (FIGS. 11D, 14C) (Gilbert et al., 2014; Wang et al., 2015). An alternative explanation for apparent IRE1α branch selectivity, other than selective activation, is the possibility that general stress caused by translocon loss impairs only the other two branches of the UPR. However, we observed CHOP upregulation in response to exogenous ER stress induced by thapsigargin treatment in cells transduced with SEC61A1-, SEC61B-, or SEC61G-targeting sgRNAs (FIG. 7E, Methods).

Cumulatively, our data suggest a selective role for the IRE1α branch of the UPR in monitoring translocon availability. Many of the strongest and most selective IRE1α transcriptional targets in the UPR epistasis experiment were translocon subunits and translocon-associated genes (FIG. 3F). Conversely, SEC61A1, SEC61G, and SEC61B were among the strongest hits in our unbiased genome-wide screen for IRE1α activation (FIGS. 4D, 4E) and repression of these genes showed preferential IRE1α pathway activation at the level of single cells (FIGS. 5D, 7A, 7B, 12E, 14A). Moreover, by RT qPCR analysis, we confirmed reciprocal upregulation of these genes in response to SEC61A1 or SEC61B repression (FIG. 14B). These results suggest a model in which IRE1α actively monitors the number of translocons (and perhaps function) and increases them as needed (FIG. 7F).

We present Perturb-seq, a platform for multiplexed profiling of perturbations with single-cell resolution, and used it to systematically dissect the mammalian UPR. Though we focused on CRISPRi, the same approach can be used to encode a wide range of perturbations, such as CRISPR cutting-mediated loss of function, gene activation, or targeted mutation (Boettcher and McManus, 2015; Komor et al., 2016). We have shown that CRISPRi can give strong, homogeneous, and simultaneous depletion of up to three targets and enables the study of essential genes. As depletion can be observed in the RNA-seq data, performance and quality of GBC identification can be directly assessed. It also has advantages when scaling to high-order combinations relative to CRISPR cutting, as genetic variability during indel formation and non-specific toxicity due to DNA cutting both increase with the number of cut sites (Boettcher and McManus, 2015; Horlbeck et al., 2016; Wang et al., 2015).

Scaling Perturb-seq to genome-scale requires overcoming some obstacles, but none appear intractable. Current techniques (Zheng et al., 2016) already allow RNA to be collected from 50,000 cells in ˜10 min, and our GBCs enable higher loading through computational removal of cell doublets. Cost per cell will decline as technologies mature, and sequencing costs can be mitigated through amplification of select targets (like our guide-mapping amplicons) or depletion of uninteresting high abundance genes (Gu et al., 2016). A more subtle point is that intermolecular provirus recombination during transduction can scramble barcode identities in pooled lentivirus preparations (Sack et al., 2016). We took careful steps to avoid this problem and expect that straightforward protocol alterations will circumvent this issue.

By far the biggest barrier we anticipate is on the analytical side. Perturb-seq generates massive amounts of intrinsically noisy data. We made some progress, using single-cell data to decouple the branches of the UPR, uncover subtle subpopulations within cells of the same type, and infer programs of gene expression using correlated expression. Along with previous successes (Jaitin et al., 2014; Klein et al., 2015; Macosko et al., 2015), and other novel analytical approaches (Dixit et al., co-submitted manuscript), large-scale analyses of single cell behavior should enable systematic understanding of the complex regulatory programs at work within cells.

Our experiments also provide insights into how the mammalian UPR senses and responds to the diverse challenges faced by the ER. A central question is why metazoan cells have evolved three independent and mechanistically distinct sensors of protein misfolding. As expected from previous work (Acosta-Alvear et al., 2007; Han et al., 2013; Lee et al., 2003; Shoulders et al., 2013), epistasis analysis using combinatorial depletion of the genes encoding PERK, ATF6, and IRE1α revealed both distinct and overlapping programs of gene expression. One of our main observations is that these branches nevertheless can operate independently, both at the bulk and single-cell levels.

Our genome-wide screens identified diverse genetic perturbations that activate IRE1α signaling, including some categories not expected from analogous yeast screens (Jonikas et al., 2009). Subjecting these hits to Perturb-seq showed that the screen in fact captured all three branches of the UPR, and that genes with similar functional roles induced the UPR in similar ways. The remarkable bifurcation in behavior we observed in cells depleted of BiP illustrated the utility of single-cell data: bulk RNA-seq would in this case describe a state that no cell actually occupies. As all cells were treated identically, the cause of such marked differences remains in question.

Perhaps the most intriguing example of branch specificity was our observation that depletion of translocon subunits led to selective activation of the IRE1α branch, which is notable in light of recent studies suggesting that IRE1α, unlike ATF6 or PERK, may act in physical association with the translocon (Plumb et al., 2015). The fact that we, in agreement with others (Shoulders et al., 2013), observed regulation of translocon expression to be uniquely under IRE1α control suggests a feedback model in which IRE1α monitors the state of translocation. Isolated IRE1α induction would enable upregulation of (or repair to) the translocation machinery without broader UPR induction, potentially forestalling responses such as cell death.

Our study of the mammalian UPR serves as a blueprint for the study of complex and overlapping transcriptional networks, in which a primary genome-wide screen serves as the input to more detailed analysis via Perturb-seq. Our success here and the parallel success in understanding dendritic cell activation (Dixit et al., co-submitted manuscript) speak well to the potential of the Perturb-seq approach to become a standard strategy for understanding regulatory interactions in the cell.

### Example II

For virtually any cell or organism, it is now possible to sequence its genome rapidly and at low cost, and to monitor the expression levels of the encoded genes. Additionally, advances in functional genomics screening technologies facilitated by RNAi and more recently by CRISPR technology greatly aid the identification of genes that contribute to specific cellular phenotypes (Shalem et al., 2015). Even with a catalog of gene-phenotype associations, however, defining the molecular function of gene products, let alone how they act together to create robust functional networks, remains a major challenge. A powerful approach to objectively and systematically identify gene function is to map genetic interactions (GIs)—pairwise measurements of how the activity of one gene modulates the phenotype of another gene. Applied broadly across many functionally diverse gene pairs, GI maps provide a signature of interactions for each gene that act as a high-resolution, quantitative phenotype, which can be used to objectively identify genes with similar functions without any a priori assumptions. The pattern of GIs can also reveal the hierarchical organization of gene products into functional complexes and pathways (Battle et al., 2010; Collins et al., 2007).

By far the most mature efforts to exploit GI maps have been in the budding yeast S. cerevisiae. Pioneering work from Boone and colleagues enabled the first large-scale measurement of GIs (Tong et al., 2001, 2004). Early GI maps demonstrated the broad utility of such efforts in enabling functional discoveries including the identification of uncharacterized protein complexes, cellular quality control and regulatory strategies and unrecognized biosynthetic pathways (Collins et al., 2007; Jonikas et al., 2009; Pan et al., 2004, 2006; Schuldiner et al., 2005; Segré et al., 2005). GI maps also revealed functional rewiring in yeast response to DNA damage or autophagy stress (Bandyopadhyay et al., 2010; Guénolé et al., 2013; Kramer et al., 2017). Additionally, comparative GI maps between S. pombe and S. cerevisiae reveal that many GIs are conserved between related yeast species, but also identify genetic repurposing of specific genes and pathways such as the Unfolded Protein Response (Frost et al., 2012; Roguev et al., 2007). More recently, hallmark papers in S. cerevisiae revealed the first and only comprehensive functional genetic landscape of a cell (Costanzo et al., 2010, 2016). Additionally, GI mapping efforts in prokaryotes, as well recent work in both fruit fly and human cells, demonstrate the general utility and enormous promise of GI maps across diverse organisms (Babu et al., 2014; Bassik et al., 2013; Du et al., 2017; Fischer et al., 2015; Gray et al., 2015; Han et al., 2017; Roguev et al., 2013; Rosenbluh et al., 2016; Shen et al., 2017; Wong et al., 2016). However, the broader goal of mapping diverse cellular processes in vertebrates remains unmet.

It is clear that systematic GI maps of human cells could be transformative tools for facilitating the systematic elucidation of the function of protein coding and non-coding genes as well as revealing higher level principles of cellular organization. Additionally, large scale GI maps can aid the design of therapeutic efforts both by identifying synthetic lethal combinations, which can enable rational design of combination therapies, as well by identifying buffering or suppressive interactions, which can provide molecular targets whose inhibition will ameliorate the consequences of genetic mutations. Finally, the nature and abundance of GIs has important implications for studying organismal evolution and “missing” inheritance from genetic linkage studies (Boyle et al., 2017; Elena and Lenski, 1997; Manolio et al., 2009;).

Despite the well-documented utility of GI maps, multiple challenges have limited large-scale GI mapping efforts in human cells. There are an enormous number of possible gene pair combinations to query (˜200 million for a mammalian cell), and strong interactions between genes typically are rare. Thus, generating quantitative genetic interaction maps require a method for robustly perturbing a given gene's functions while avoiding heterogeneity and off-target effects. Additionally, for large numbers of gene pairs, one must be able to precisely measure the effect of each genetic perturbation and quantitatively evaluate the observed defect for a gene pair relative to that expected from the phenotypes of the individual perturbations. These challenges have been mitigated by pre-selecting smaller subsets (˜20-fold or fewer gene pairs than in the present study) of functionally related genes (e.g., involved in chromatin-regulation, toxin resistance, regulators of β-catenin activity, or cancer biology) (Bassik et al., 2013; Du et al., 2017; Han et al., 2017; Roguev et al., 2013; Rosenbluh et al., 2016; Shen et al., 2017; Wong et al., 2016). However, it remains unresolved whether a GI map of diverse human genes can generate a GI signature enabling one to cluster genes by function and assign function to poorly characterized genes. For example, the most expansive screen to date, which involved a subset of the “druggable genome” comprising 207 functionally diverse genes, identified rare interactions but did not yield sufficiently rich GI signatures to cluster genes into a GI map or to systematically assign function to poorly characterized human genes—correlations between sgRNAs targeting the same gene were virtually indistinct from correlations between random sgRNAs (Han et al., 2017). Thus, whether it will be possible to construct large-scale, diverse GI maps in human cells similar to yeast GI maps, let alone how to accomplish this, remains an open question.

Described herein is a new mammalian GI mapping platform, based on CRISPR interference (CRISPRi), in which the expression of targeted genes is specifically repressed using a catalytically dead version of Cas9 (dCas9) fused to a KRAB transcriptional repression domain, allowing for precise and homogenous gene knockdowns (Gilbert et al., 2013, 2014; Horlbeck et al., 2016a). We present a combined experimental and analytic framework for high-precision ultra-rich GI mapping and apply this platform to create a high-content large-scale GI map of functionally and spatially diverse human genes. Our GI map contains 1,044,484 sgRNA pairs targeting 222,784 gene pairs, which increases by a factor of four the number of genetic interactions measured in human cells. Starting with a highly functionally diverse set of genes, our GI platform reveals high-content GI signatures that enable us to group related genes and assign function to even poorly characterized genes in an unbiased manner. Our CRISPRi GI map also classifies known and new GIs in pathways and protein complexes across diverse cellular processes, revealing unexpected biological principles and demonstrating that this method is well suited for systematic functional analysis of mammalian cells. We further show that GI maps can be used to identify robust genetic suppressors and synthetic sick/lethal (SSL) gene pairs, which point to therapeutic strategies for human diseases. Our maps are both a broad resource and a demonstration that large-scale CRISPRi GI maps can systematically elucidate how sets of genes encode the biology of protein complexes, pathways and organelles in human cells, providing both the motivation and an experimental and analytic framework for constructing a GI map of the entire human cell.

### Methods

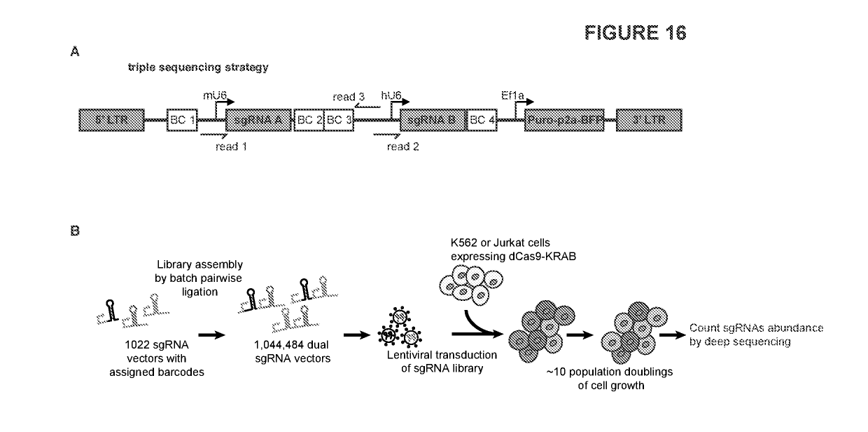

Plasmid design and construction The GI sgRNA library vector is a modified version of the sgRNA lentiviral plasmid that was previously described (FIG. 16) (Horlbeck et al., 2016a). In the final GI library sgRNA vector, the 5′ sgRNA is expressed from a modified mouse U6 promoter while the 3′ sgRNA is expressed from a modified human U6 promoter (FIG. 16). Both sgRNAs expressed from this vector employ the same optimized S. pyogenes sgRNA constant region. The GI library sgRNA vector also encodes 4 randomized 16 base pair DNA barcodes allowing us to measure vector recombination by Illumina sequencing (FIG. 16A). The GI lentiviral sgRNA construct co-expresses BFP and a puromycin resistance cassette separated by a T2A sequence from a Ef1 Alpha promoter. The lentiviral sgRNA vectors for the dual color competition assay to confirm GI phenotypes are previously described but briefly each vector encodes a modified mouse U6 promoter that drives expression of the sgRNA described above as well as either GFP or BFP and a puromycin resistance cassette separated by a T2A sequence from an Ef1Alpha promoter.

We used previously described lentiviral vectors to express the CRISPRi dCas9-KRAB protein (Gilbert et al., 2013). The CRISPRi fusion encodes mammalian codon optimized S. pyogenes dCas9 (DNA 2.0) fused at the C-terminus with two SV40 nuclear localization sequences (NLS), BFP and the Kox1 KRAB domain expressed from either the SFFV or Ef1Alpha promoter.

GI library design The gene set was obtained from all genes that had a growth phenotype (γ) less than −0.1 and greater than −0.3 in a CRISPRi v1 growth screen (Gilbert et al., 2014). Genes were further filtered to require that all genes had a “discriminant score” greater than 30 in our sgRNA activity dataset (Horlbeck et al., 2016b), to ensure that multiple sgRNAs targeting each gene were active. Two sgRNAs targeting each gene were selected using the top two sgRNAs by activity score; in arbitrary cases, the third sgRNA was also included to assess the improvement in gene GI measurement with additional sgRNAs/gene. CRISPRi v1 sgRNAs were of variable length (18-25 bp); for the GI map, all were standardized to G[N191]NGG as with our CRISPRi v2 libraries (Horlbeck et al., 2016a). sgRNAs targeting several genes in complexes of interest (e.g., EMC) were included manually.

GI library cloning Our GI CRISPRi libraries were prepared by library cloning protocols similar to those previously described for sgRNA libraries with the following differences. Our final GI sgRNA library vector is assembled in four steps.

1. We first cloned the sgRNA constant region and two 16 base pair random DNA barcodes into a modified pSICO vector PCR. The PCR product and parental vector were restriction digested with Xba1/BamH1, gel purified and the appropriate fragments were ligated together. The 5′ and 3′ barcode are upstream and downstream of the sgRNA constant region. The randomized barcodes were encoded on oligonucleotides purchased from IDT. This starting vector lacks a U6 promoter.

2. A starting pool of oligonucleotides encoding ˜1000 sgRNAs targeting ˜500 genes (2 sgRNAs/gene) was synthesized by Agilent. The library was amplified by PCR, the library and library vector were digested with either BstX1 and Blp1, and then ligated and cloned as a pooled library into the barcoded promoterless vector described above. We Sanger sequenced 4000 bacterial colonies from the pooled library of 1000 sgRNAs that we had cloned. We retained DNA and glycerol bacterial stocks from each sequenced colony to create an arrayed library of 750 unique sgRNAs. To complete our arrayed GI library we then filled in the remaining 250 sgRNAs desired for our GI map by ordering arrayed oligos and cloning sgRNAs in an arrayed fashion as previously described (Liu et al., 2017). By Sanger sequencing all 1022 sgRNA plasmids we are able to ensure that out library should have no mutations or errors and to assign each sgRNA in the library with two unique barcodes. We then pooled 1022 sgRNA plasmids targeting 472 genes (1-3 sgRNAs/gene) including 18 negative control sgRNAs at even stoichiometry. We used Illumina sequencing to ensure our pooled library was intact.

3. We next cloned either a modified human or modified mouse U6 promoter into our pooled sgRNA library of 1022 plasmids creating two libraries where each vector encodes 1 U6-sgRNA cassette and 2 unique barcodes. We restriction digested parental mouse or human U6-sgRNA vectors or the GI library library with Xho1/BstX1 and then ligated the appropriate fragments together. We used Illumina sequencing to ensure each library was intact.

4. We then restriction digested the mouse U6-sgRNA library with Avr-II and Kpn-1 and the human U6-sgRNA library with Xba1 and Kpn-1, isolated the appropriate DNA fragment and ligated these two libraries together creating our final GI sgRNA library vector that encodes 2 sgRNAs expressed from the 5′ position by the mouse U6 promoter and the 3′ position by the human U6 promoter and 4 unique DNA barcodes (FIG. 16A). By constructing an arrayed library and then pooling the library evenly each sgRNA in our intermediate library assembly steps is well represented enabling us to randomly ligate the two intermediate libraries with 1015 sgRNAs together to create a final pool of 965,000 sgRNAs but maintain even sgRNA representation within the library. We used Illumina sequencing to ensure the final library was assembled properly. We note that even with this strategy because we cloned this library at only 25-fold coverage we lost a number of sgRNAs.

Cell culture, DNA transfections, and viral production and construction of CRISPRi cell lines HEK293T cells used for packaging lentivirus were maintained in Dulbecco's modified eagle medium (DMEM) in 10% FBS, 100 units/mL streptomycin and 100 μg/mL penicillin with 2 mM glutamine. K562 and Jurkat cells were grown in RPMI-1640 with 25 mM HEPES and 2.0 g/L NaHCo3 in 10% FBS, 2 mM glutamine, 100 units/mL streptomycin and 100 μg/mL penicillin (Gibco). Lentivirus was produced by transfecting HEK293T with standard packaging vectors using TransIT®-LTI Transfection Reagent (Mirus, MIR 2306). Viral supernatant was harvested 72 hours following transfection and filtered through a 0.45 μm PVDF syringe filter.

To construct CRISPRi cell lines, K562 or Jurkat cells were lentivirally transduced to express dCas9-BFP-KRAB from the SFFV or Ef1a promoter respectively (FIG. 16B). To construct a pure polyclonal populations K562 CRISPRi cell line, we sorted by flow cytometry using a BD FACS Aria2 for stable BFP signal which marks dCas9-BFP-KRAB expression. For the CRISPRi Jurkat cell line, were isolated and analyzed single cell clones as described previously.

High-throughput pooled GI screening CRISPRi K562 or Jurkat cell lines were infected with sgRNA libraries as previously described (Gilbert et al., 2014). The lentiviral infection was scaled to achieve an effective multiplicity of infection of less than one lentiviral integration per cell as measured by BFP signal encoded on the GI sgRNA library vector. Throughout the GI screen, cells were maintained at a density of between 500,000 and 1,500,000 cells/mL continually maintaining a library coverage of at least 500 cells per sgRNA except at the initial infection where we infected 250 cells per sgRNA. Two days after lentiviral infection, cells were selected with 0.75-1 μg/mL puromycin (Sigma) for 2 days, and recovered with addition of fresh media ˜24-48 hour recovery. For the screen, populations of K562 or Jurkat cells expressing this GI library were harvested at the outset of the experiment or after ˜10 population doublings. Two biological replicates of each screen were performed. Genomic DNA was harvested from all samples; the sgRNA-encoding regions were then amplified by PCR and sequenced on an Illumina HiSeq-2500 or 4000 using custom primers with previously described protocols at high coverage. From this data, we quantified the frequencies of cells expressing different sgRNAs in each sample.

GI Map Data Analysis

Sequence alignment Triple sequencing raw data was generated in the form of 3 parallel FASTQ files corresponding to Read 1, Read 2, and Read 3 (see FIG. 16B), and were processed using custom scripts in Python 2.7. Read 1 and read 2 were stripped to yield only the 19 bp corresponding to the N19 of sgRNA A and B, respectively, and each were mapped separately to the sgRNAs included in the GI map library. Read 3 was reverse complemented and stripped to yield two 16 bp barcodes corresponding to BC2 (the downstream barcode of sgRNA A) and BC3 (the upstream barcode of sgRNA B), and each were mapped separately to the list of downstream or upstream barcodes included in the GI map library. All mappings tolerated up to one mismatch; typically 98% of sgRNAs mapped to the library and 50-70% of barcodes mapped due to degradation of sequencing quality in the reverse reads.

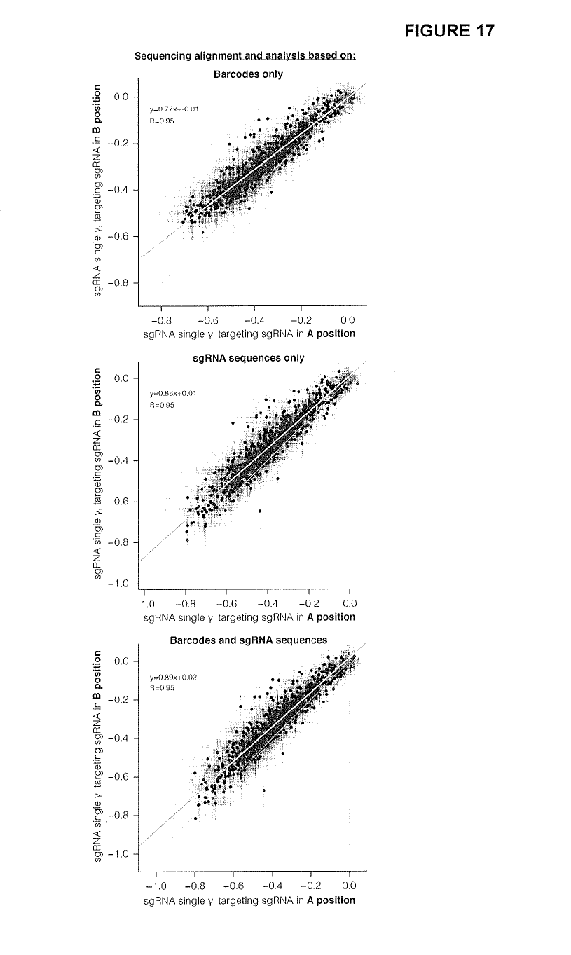

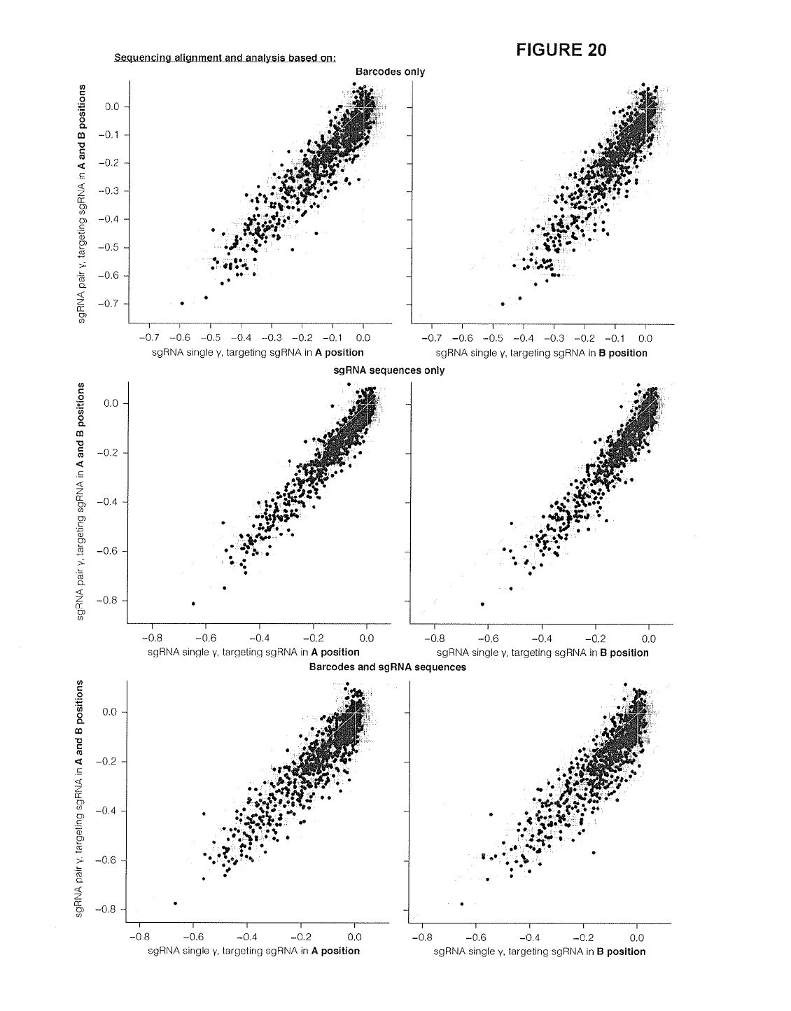

For “sgRNAs only” and “barcodes only” analysis (FIGS. 17, 18, 19, 20), sgRNA A/B pair representation was counted from the sgRNA reads or the barcode reads without further filtering. For triple sequencing-based analysis (all other data), the identity of sgRNA A was required to match BC2 and sgRNA B was required to match BC3 before including that sequence in the count of sgRNA A/B pair representation. In K562, ˜5% of sgRNA A and BC2 reads did not match while ˜16% of B/BC3 reads did not match, proportional to the distance between those elements in the lentiviral vector. In Jurkat, the mismatch rates were ˜10% and ˜30%, respectively, consistent with the hypothesis that these mismatches arise due to template switching during lentiviral reverse transcription, a process that occurs within the infected cell and is modified by host cell factors (Sack et al., 2016). Of note, K562 T0 read counts only use barcodes as we developed the triple sequencing strategy after initial experiments with barcode-only sequencing and no further sample was available for re-processing.

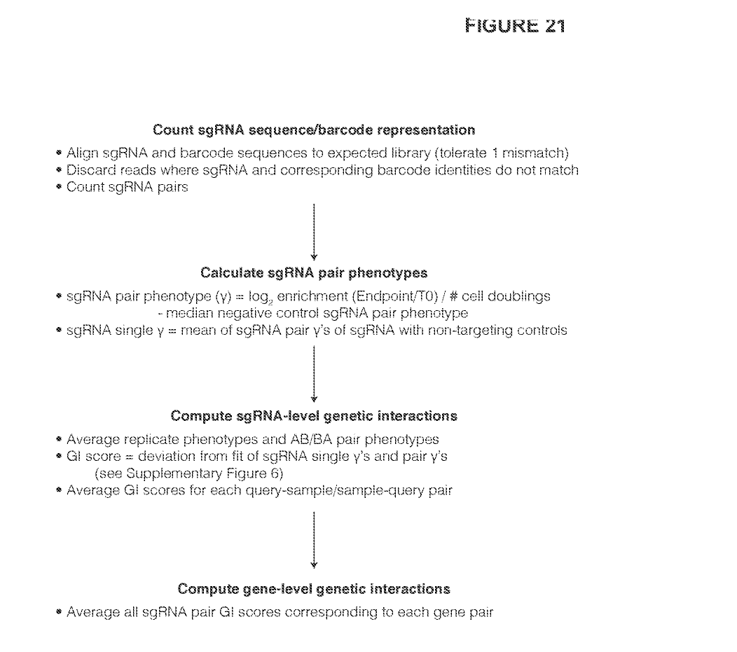

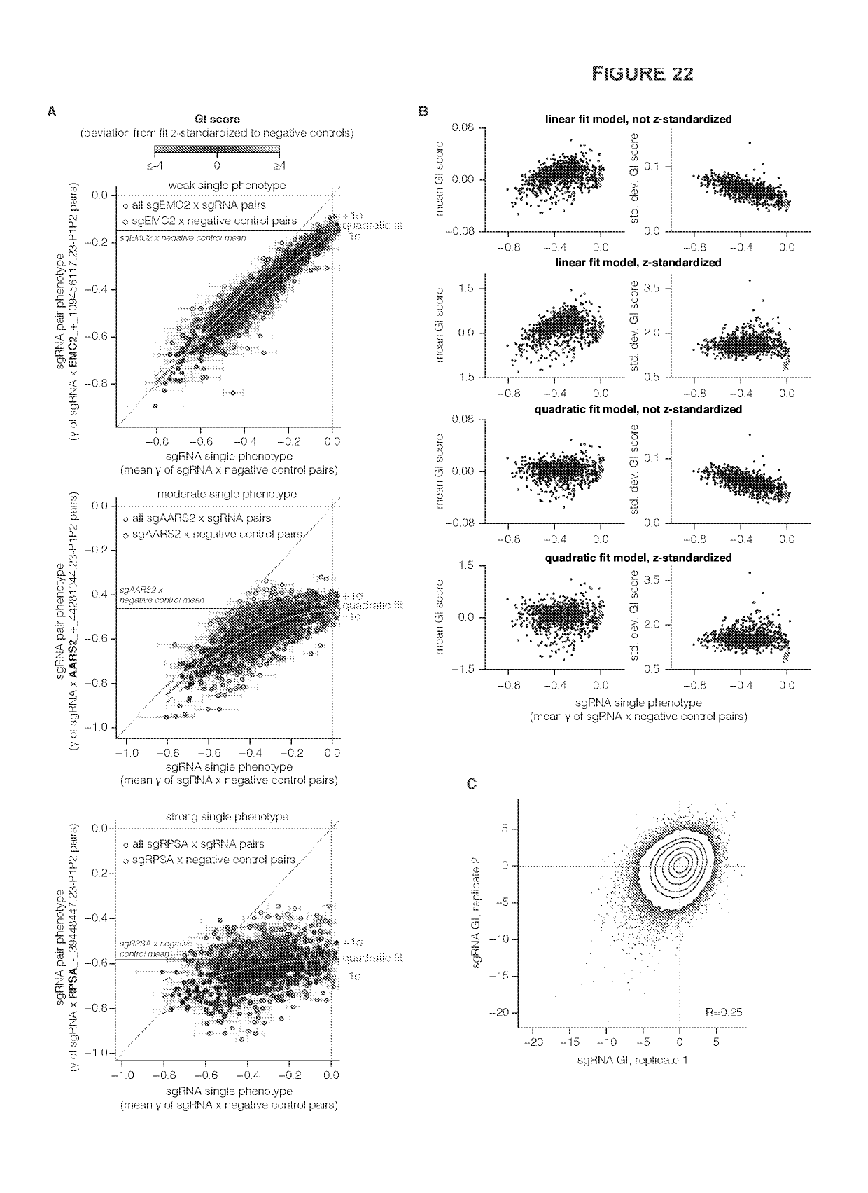

Calculating sgRNA pair phenotypes Phenotypes for sgRNA pairs were calculated similarly to previously described approaches for single sgRNA screens using custom scripts in Python 2.7 (GI analysis pipeline summarized in FIG. 21) (Horlbeck et al., 2016a). For a given screen replicate, T0 and endpoint sgRNA A/B pair counts were first filtered by requiring that all single sgRNAs (in A or B position) had a median representation of at least 35 reads in the endpoint sample across all pairs in which that sgRNA was a member. This filter was imposed because sgRNAs that depleted significantly by the end of the screen did not yield robust GI measurements (FIG. 22A), and also enabled us to maintain a square GI map for downstream analysis. Similarly, because read count differences in poorly represented sgRNA pairs could result in significantly different GI measurements, a pseudocount of 10 was applied to all sgRNA pair counts in both T0 and endpoint samples. Log₂enrichment of pair representation in endpoint relative to T0 samples was then calculated as previously described. The median enrichment for pairs in which both sgRNAs were non-targeting controls was set to zero by subtraction, and growth phenotype (γ) was computed by dividing the log₂ enrichment by the number of doublings between T0 and endpoint (K562 Rep1=6.91, K562 Rep2=7.61, Jurkat Rep1=6.15, Jurkat Rep2=6.44). sgRNA pair phenotypes was calculated.

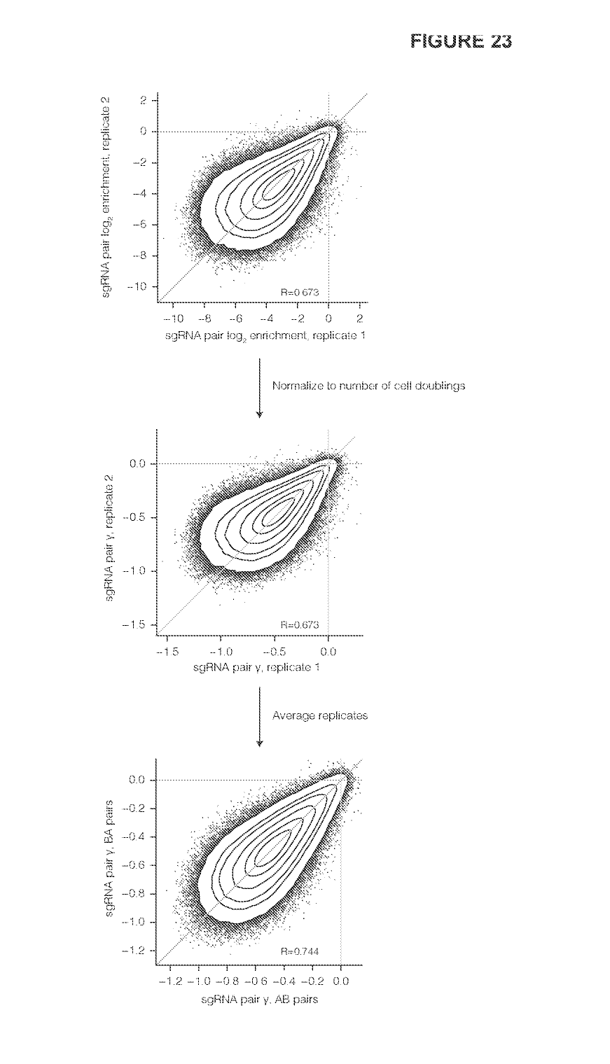

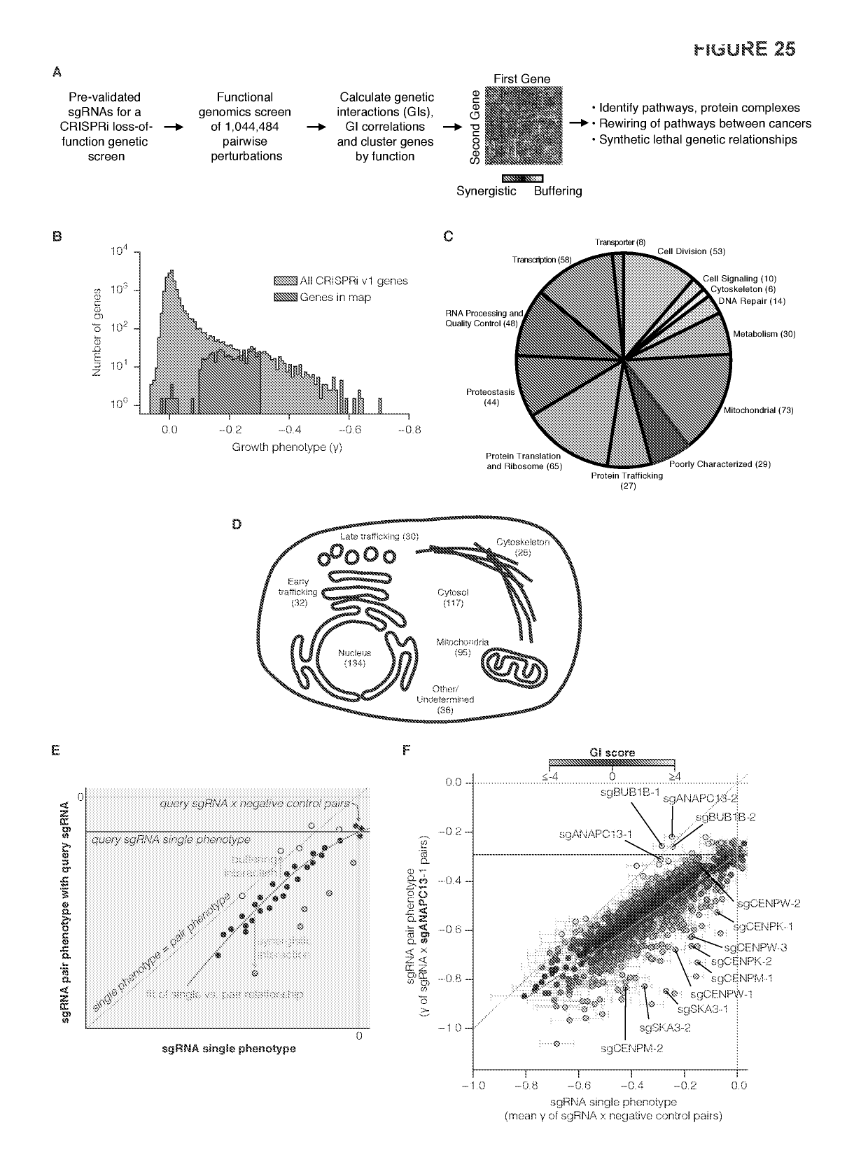

Computing genetic interaction scores Replicate pair phenotypes were averaged (except for replicate-specific analyses) and then sgRNA A/B and B/A pairs were averaged (FIG. 23) to obtain a symmetric phenotype matrix of sgRNA pair phenotypes with reduced measurement noise. sgRNA single phenotypes were calculated for each sgRNA from the mean phenotype of that sgRNA paired with non-targeting control sgRNAs. Each sgRNA was then treated as the query sgRNA for calculation of GIs (FIGS. 25D and 22A-B). A quadratic fit of sgRNA single phenotypes and sgRNA pair phenotypes with the query sgRNA, with y-intercept set to the single phenotype of the query sgRNA, was calculated using the Optimize module of SciPy 0.17.0. GIs were then calculated by subtracting the expected pair phenotype (determined by the quadratic fit function value at the given sgRNA single phenotype) from the measured sgRNA pair phenotype. For each query sgRNA these GIs were z-standardized to the standard deviation of the negative control/query pair GIs. Finally, the query/sample and sample/query GIs for each sgRNA pair were averaged to obtain a symmetric matrix of sgRNA GIs. Gene-level GIs were calculated by simply averaging all sgRNA pairs targeting a given gene pair. For analyses requiring “negative control genes,” all possible combinations of two non-targeting control sgRNAs were averaged as with sgRNAs targeting the same gene.

Analysis of GI Map

Clustering and visualization To cluster, visualize, and explore sgRNA-level and gene-level GI maps, symmetric GI matrices excluding non-targeting controls were clustered with average linkage hierarchical clustering using uncentered Pearson correlation in Cluster 3.0 (de Hoon et al., 2004) and the output files were loaded in Java TreeView 1.1.6r4 (Saldanha, 2004) as previously described (Bassik et al., 2013; Kampmann et al., 2013).

GI correlations were calculated using NumPy 1.12.1. STRING interactions were obtained from the experimentally validated set from version 10.0, and expressed using the STRING-specified confidence thresholds (low>=0.15, medium>=0.4, high>=0.7, highest>=0.9) (Szklarczyk et al., 2017). GI correlation network in FIG. 3D was generated in Cytoscape 3.5.1 (Smoot et al., 2011) using equally-weighted edges between all gene pairs with GI correlation>=0.6, with edge length set with force-directed layout. Neighbor TSS analysis was performed as in (Liu et al., 2017) based on the closest of all P1/P2 TSS annotation pairs from (Horlbeck et al., 2016a), and nearest neighbor identities and distances were determined.

Annotation of gene product function and localization For each gene in the map, we annotated gene function using the Entrez Gene Summary and UniProt databases. To generate an unbiased annotation of the localization of the protein products of the genes included in the GI map (e.g. FIG. 25C), we leveraged two recently published datasets: the mass-spectrometry-based Map of the Cell (Itzhak et al., 2016) and the immunofluorescence-based Cell Atlas (Thul et al., 2017). Together, these annotations contained localization predictions for the large majority of genes in the GI map and could be used to refine localization in cases where one dataset gave ambiguous calls. To obtain a single “high level” localization for the product of each gene, Map of the Cell predictions were used wherever available unless the prediction was “Large Protein Complex” or “No Prediction.” If no prediction was available, the first listed and best available Cell Atlas prediction (of “Approved,” “Supported,” and “Validated”) was used. If no prediction was available, the gene localization was listed as “Undetermined.” To generate a high-level annotation, these calls were collapsed to cell compartments as follows:
Early trafficking: Golgi apparatus, ER_high_curvature, Golgi, Ergic/cisGolgi, ER
Cytosol: Cytoplasmic bodies, Cytosol
Late trafficking: Cell Junctions, Peroxisome, Vesicles, Endosome, Plasma membrane
Mitochondria: Mitochondria, Mitochondrion
Other/Undetermined: Undetermined
Nucleus: Nucleoli fibrillar center, Nuclear bodies, Nuclear pore complex, Nucleus, Nuclear membrane, Nucleoli, Nucleoplasm, Nuclear speckles
Cytoskeleton: Midbody ring, Focal adhesion sites, Microtubules, Actin filaments, Midbody,
Cytokinetic bridge, Centrosome, Intermediate filaments

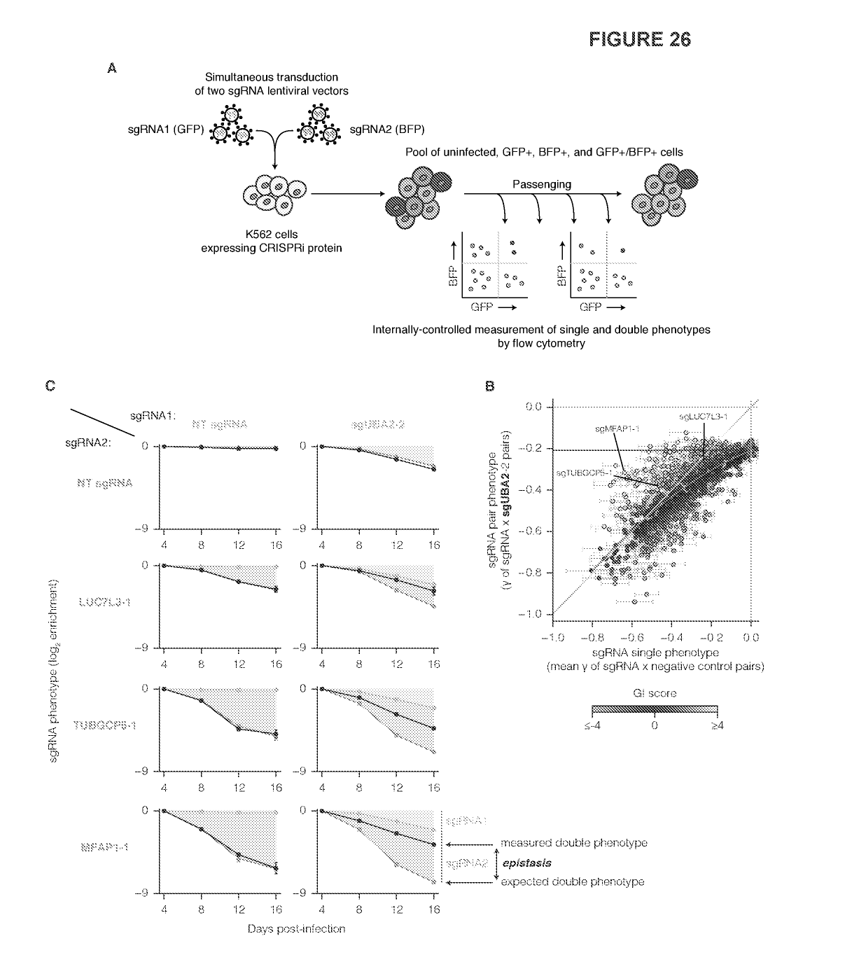

Individual re-test of GI phenotypes, chemical genetics and CRISPRi transcript repression Individual phenotype re-test experiments for sgRNA pair phenotypes from the GI screens were performed as dual color competitive growth experiments on a partially transduced population of CRISPRi K562 or Jurkat cells. Briefly, cells were co-transduced at ˜5-60% infection with two lentiviral vectors marked with either BFP or GFP each encoding a single sgRNA. This assay enables us to track uninfected cells, cells that express each single sgRNA or cells that express a pair of sgRNAs within one internally controlled sample by flow cytometry over time to quantify how each sgRNA or pair of sgRNAs influences cell proliferation (FIG. 26A). Three or four days following infection, cells were counted and seeded in 24 well plates at 0.25 million cells/mL and diluted 1:2 or 1:4 every 2 or 3 days as cells reached ˜1,000,000/mL. Duplicate or triplicate samples for each GI re-test experiment were grown under standard conditions described above. The absolute cell number and percentage of cells that express BFP or GFP (indicating sgRNA expression) was measured for each sample at the indicated time points. Cells were treated with 4 or 6 μM lovastatin (Tocris) or a DMSO control as indicated every 3 or 4 days over the time course of the experiment.

To determine the amount of gene knockdown for individual sgRNAs, we partially transduced cells at 20-40% with individual sgRNAs, and then at 2 or 3 days post infection cells were selected with 3 μg/mL puromycin. Cells were allowed to recover from selection and then were harvested for RT-qPCR.

Quantitative RT-PCR Cells were harvested and total RNA was isolated using the Direct-zol-96 RNA (Zymo Research), according to manufacturer's instructions. RNA was converted to cDNA using SuperScript III reverse transcriptase under standard conditions with oligo dT primers and RNaseOUT (ThermoFisher). Quantitative PCR reactions were prepared with a 2× SYBR Select master mix according to the manufacturer's instructions (ThermoFisher). Reactions were run on a QuantStudio7 thermal cycler (Applied Biosystems).

Western Blotting K562s were harvested by centrifugation and resuspended in lysis buffer (1% Triton-X, 0.15M NaCl, 1 mM EDTA, 50 mM Tris-HCl pH 7.5, 1× Halt Protease Inhibitor Cocktail (Thermo Fisher Scientific), 1× Phosphatase Inhibitor Cocktail A and B (Biotool Chemicals)). Cells were lysed by vortexing for 1 min, and incubating on ice for 30 min. Lysate was clarified by centrifugation at 10,000 g for 30 min. Protein concentration was measured by the Pierce BCA Protein Assay (Thermo Fisher Scientific). Cell lysates were denatured at 100° C. for 5 min in 1× NuPAGE LDS Sample Buffer (Thermo Fisher Scientific). Proteins were separated on a Bolt 4-12% Bis-Tris gel (Thermo Fisher Scientific), transferred to a TransBlot Turbo Mini-size nitrocellulose membrane (Bio-Rad) according to the manufacturer's instructions, blocked with Odyssey Blocking Buffer (LiCor), and subsequently probed. Chk1 was detected with the Chk1 mouse antibody (Cell Signaling #2360, 1:1000 dilution). Phospho-Chk1 was detected with the Phospho-Chk1 (Ser345) rabbit antibody (Cell Signaling #2348, 1:1000 dilution). Actin was detected with the anti-β-Actin mouse antibody (Sigma Aldrich #A5441, 1:5000 dilution). IRDye 680RD Goat anti-Rabbit (Odyssey) and IRDye 800CW Donkey anti-Mouse (Odyssey) secondary antibodies were used at a 1:5000 dilution. All blots were visualized using the Odyssey C1× Li-Cor systems.

### Results

A CRISPRi Platform for GI Mapping in Human Cells

We devised a strategy for creating draft loss-of-function GI maps in human cells using CRISPRi-expressing cells transduced with dual sgRNA lentiviral vectors to screen for pairwise sgRNA phenotypes (FIG. 25A). We chose to use CRISPRi for this GI platform because we have shown we can silence expression of most genes encoded by the human genome in a highly specific manner and because CRISPRi has several unique properties that facilitate GI mapping efforts (Gilbert et al., 2013, 2014; Horlbeck et al., 2016a; Liu et al., 2017; Mandegar et al., 2016; Qi et al., 2013). CRISPRi acts at the level of transcription rather than DNA editing and so, unlike CRISPR/Cas9, CRISPRi does not produce in-frame indels of unknown functional consequence. In pooled functional genomic screens, in-frame indels have been shown to generate phenotype heterogeneity that will be compounded by simultaneously targeting more than one gene (Shalem et al., 2015; Shi et al., 2015). As set forth above, in Example I and in Adamson et al., we have shown by population and single-cell RNA sequencing that CRISPRi can be used to effectively, specifically, and homogeneously silence the expression of up to 3 genes simultaneously, which should enable robust GI mapping efforts. Lastly, CRISPRi activity does not generate DNA double stranded breaks that activate a DNA damage response and can lead to non-specific toxicity phenotypes (Aguirre et al., 2016; Munoz et al., 2016; Wang et al., 2015).

To construct a GI map, we developed a dual sgRNA lentiviral vector that enabled us to robustly silence pairs of genes and then track each perturbation in a pooled CRISPR screen. Our design employed a dual-barcoded vector encoding two sgRNAs expressed from a modified mouse and human U6 promoter, enabling repression of two genes from a single lentiviral integration (FIG. 16A). Recombination due to RNA template switching by the HIV reverse transcriptase or DNA template switching by DNA polymerase during PCR has been shown to confound quantitative genetic analysis by scrambling nucleic acid information (Du et al., 2017; Han et al., 2017; Sack et al., 2016). We developed a new sequencing strategy and analysis pipeline we named “triple sequencing” that sequences both the barcodes and the sgRNAs encoded by each DNA molecule, allowing us to identify and discard in silico all recombination events (FIG. 16A).

A GI Map of Diverse Cellular Processes

We constructed a large loss-of-function GI map targeting genes identified in a CRISPRi screen as conferring a growth disadvantage when knocked down in K562 cells (Gilbert et al., 2014). This enabled us to construct the map using sgRNAs that were all experimentally validated as active from previous CRISPRi screens. With the exception of several selected genes, the vast majority of the targeted genes were chosen in an unbiased fashion based on their moderate growth phenotype (FIG. 25B). Analysis of the gene set in our map reveals that the encoded gene products represent diverse cellular processes that are localized to all major intracellular cellular compartments (FIG. 25C-D). We used a custom cloning strategy to construct a dual sgRNA library of 1,044,484 pairwise CRISPRi genetic perturbations targeting 222,784 gene pairs (472 genes×472 genes) (FIGS. 25A and 16A-B).

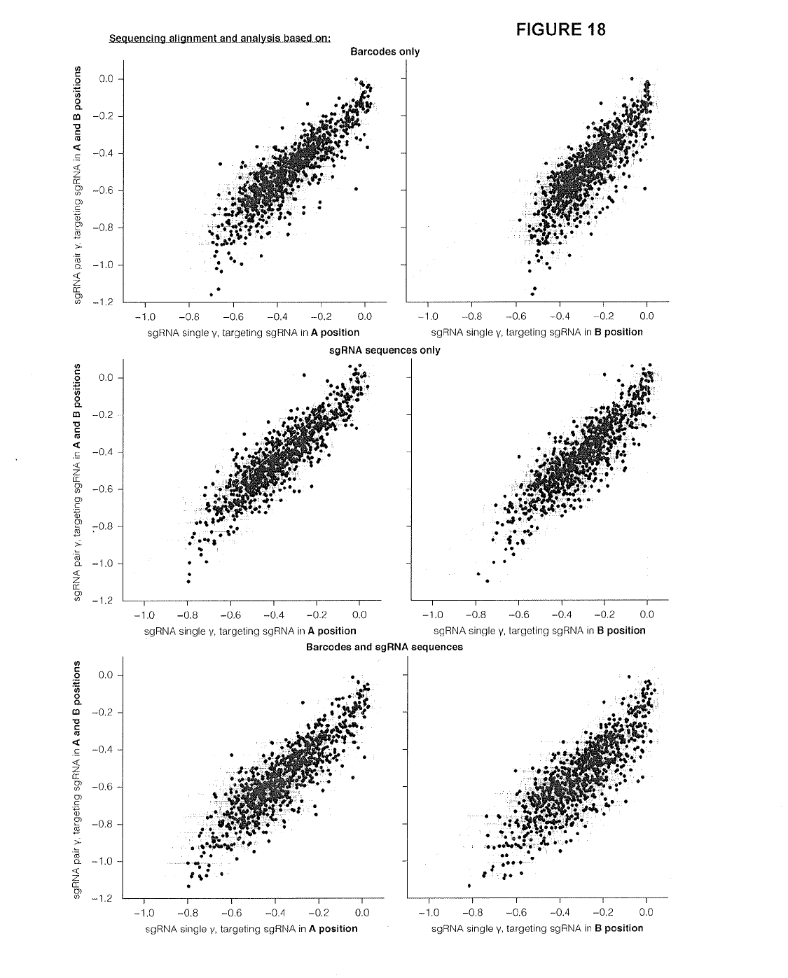

We transduced K562 cells stably expressing dCas9-KRAB with our GI library in replicate and conducted two independent cell growth screens over ˜10 cell population doublings to measure how each sgRNA pair perturbs cell proliferation (FIG. 16B). Using triple sequencing, we measured the growth phenotype (γ) of sgRNA pairs based on their relative abundances at the start and end of the screen, normalized to the number of cell doublings (FIG. 23). Our triple sequencing analysis clearly revealed recombination between the A and B sgRNA positions in our vector, with ˜5% of sgRNA A and the corresponding barcode mismatched and ˜16% of sgRNA B and barcode mismatched, proportional to the distance between those elements in the lentiviral vector. We used this strategy to remove recombination products, thus correcting this artifact that limited the dynamic range of our screen phenotypes (FIGS. 17, 18).

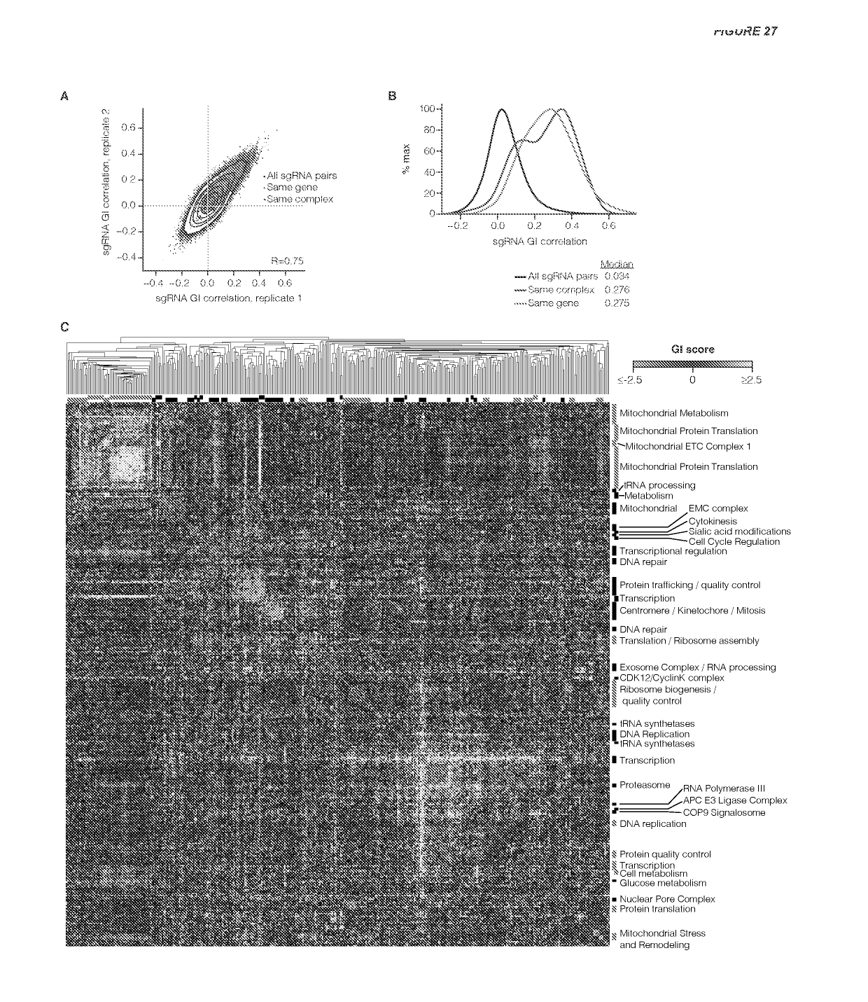

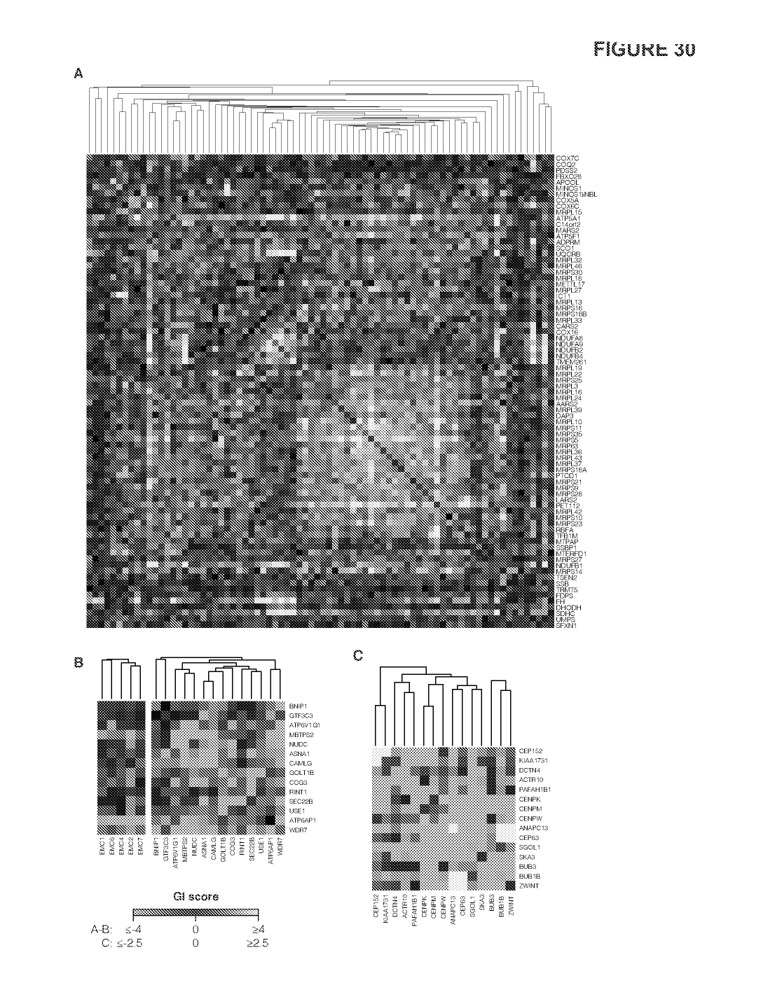

We analyzed the phenotypes from our screen through a custom pipeline to calculate sgRNA- and gene-level interactions (FIG. 21). We computed sgRNA interactions based on a GI paradigm previously established for yeast GI screens as well as a pooled shRNA GI screen (Bassik et al., 2013; Collins et al., 2006, 2007; Jonikas et al., 2009; Kampmann et al., 2013; Schuldiner et al., 2005). For a given “query” sgRNA, we plotted the relationship between each sgRNA's single phenotype and the double phenotype when paired with the query sgRNA. Interactions were then calculated based on the deviation of the observed double-sgRNA phenotype from the typical phenotype expected based on the phenotype of the two individual sgRNAs (FIGS. 25E-F and 22A-B). We classify strong deviations from the expected phenotype as buffering or synergistic (FIG. 25E). Buffering GIs most commonly reveal genes that function in a linear pathway or a protein complex while SSL GIs identify pairs of genes acting in parallel to support cell viability. In our dataset, we found that a quadratic fit best captured these relationships, as the phenotype strength of the query sgRNA greatly affected the shape and sequencing representation of the single vs. double relationship (FIG. 22A-B). Five observations argue for the validity and reproducibility of the measured GIs. First, GIs for each replicate screen are well correlated (FIG. 22C). Second, analyzing the correlations between sgRNA GI profiles, we found these show very high concordance across independent replicate screens (FIG. 27A). Third, sgRNAs targeting the same gene correlated well compared to the background of all sgRNAs pairs (i.e. the median same-gene sgRNA correlation is 8-fold stronger than background; FIG. 27B). Fourth, sgRNAs targeting genes in the same biological complex are also well correlated, with the caveat that for extremely sick sgRNA pairs, it is difficult to accurately measure a GI signature (FIGS. 27A-B, 28, 29A). Finally, we experimentally validated a number of weak, medium and strong buffering GIs as well as several synergistic and neutral interactions using a custom lentiviral two-color internally-controlled growth assay, which enables us to knockdown expression of each gene or gene pair and then track each of the knockdown phenotypes over time (FIG. 26A). We found that all gene pairs interacted as expected, confirming that the results from our GI map robustly identify sgRNA pairs that target interacting genes (FIGS. 26, 30).

GI maps Cluster Genes by Function Enabling Unbiased Characterization of Genes with Poorly Characterized Function.

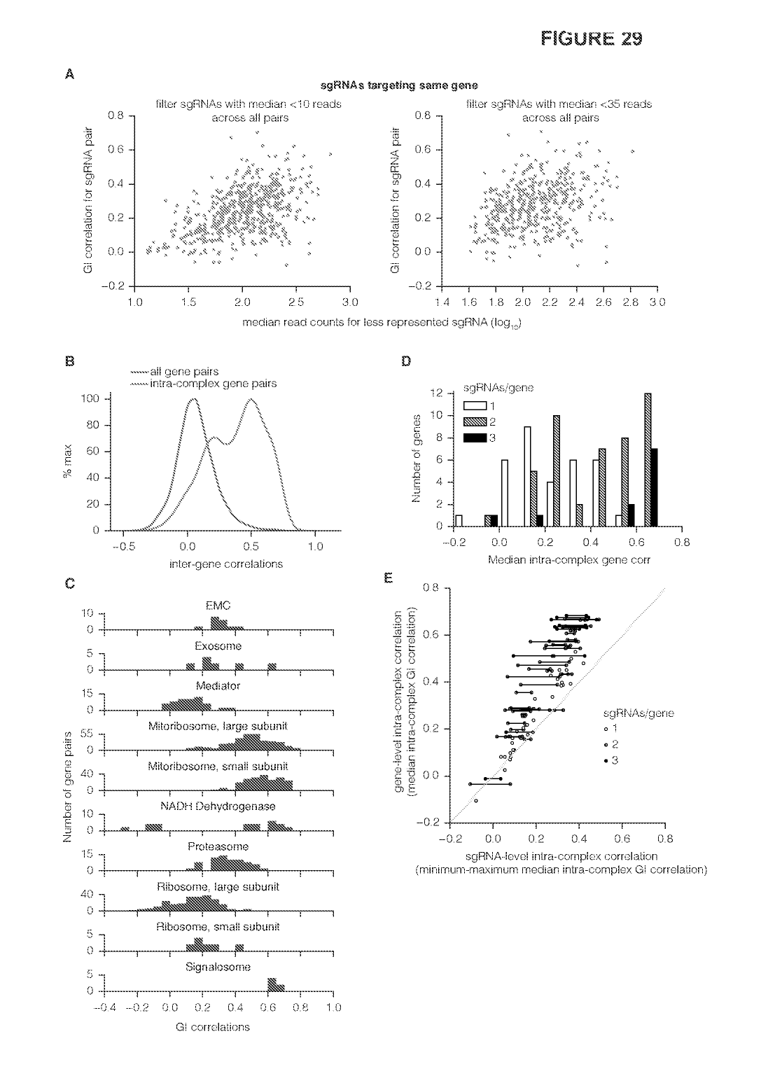

To construct a gene-level GI map, we first averaged interactions for all corresponding sgRNA pairs (1-3×1-3, depending on the genes) targeting a given gene pair to generate gene-level interactions. As with sgRNA-level interactions, intra-complex gene pairs were much more highly correlated than background (FIG. 29B-C). Intra-complex correlations vary by complex, which may be due to the challenge of GI mapping highly essential gene pairs or may point to functionally distinct complex subunits or sub-complexes (Bassik et al., 2013; Collins et al., 2007; Shi et al., 2017; Simsek et al., 2017). Our analysis shows that GI maps containing just 1 sgRNA targeting each gene could be informative (FIG. 29D-E), but inclusion of multiple sgRNAs will boost signal-to-noise at the cost of larger double-sgRNA libraries.

Strikingly, hierarchical clustering of our GI map demonstrates the power of GI mapping for the functional characterization of human genes of diverse or unknown functions (FIG. 27C). At least 37 functionally coherent clusters (containing 2-35 genes) emerge from this analysis. These recapitulated known biology, and many gene clusters also incorporated genes with poorly annotated function (FIG. 27C). Prominent highlights include a mitochondrial supercluster with clear subclusters functionally defining mitochondrial metabolism genes, mitochondrial protein translation and Complex 1 of the Electron Transport Chain, indicating that our GI mapping platform can reveal interconnected functional processes within an organelle (FIG. 30A). We also observe two large clusters of genes involved in the secretory pathway and in mitosis (FIG. 30B-C). Within the mitotic cluster, gene products required for centromere function including CENPK/CENPM/CENPW are accurately segregated from BUB1B/BUB3 that function in the mitotic checkpoint. Analysis of our GI map demonstrates that gene clustering can be driven by synergistic and/or buffering interactions. We note that repression of several of the ER Membrane Complex (EMC) genes does not have a primary growth phenotype; however all EMC genes cluster well together (FIG. 30B). This provides evidence that GI maps can assign functional significance to genes even in the absence of primary phenotypes commonly used to evaluate gene function such as cell growth.

GI Mapping Reveals a High Degree of Unannotated Gene Function in Human Cells

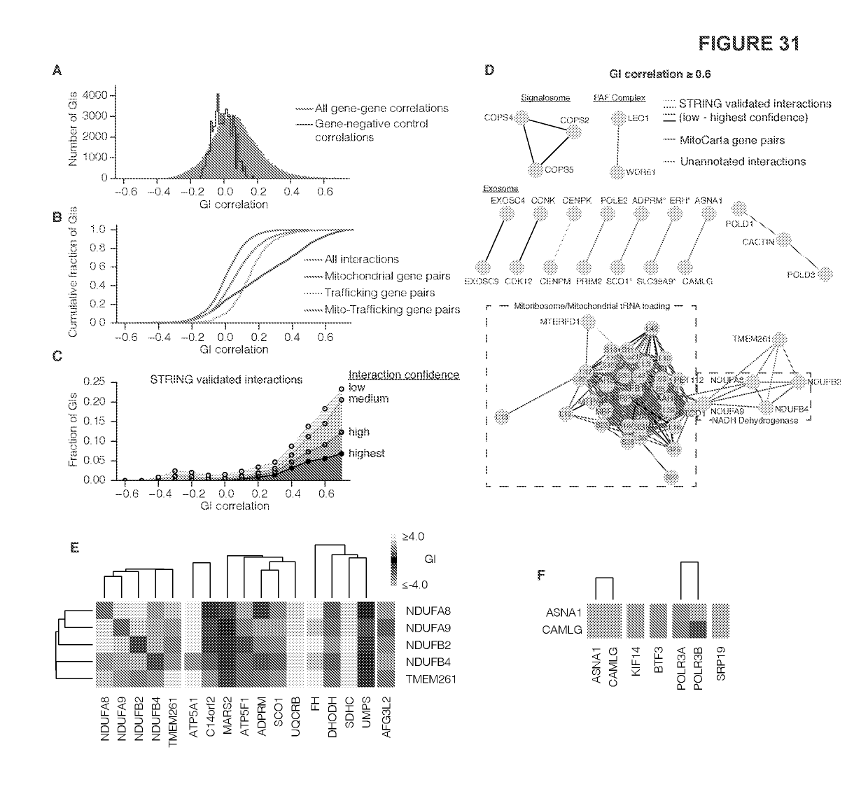

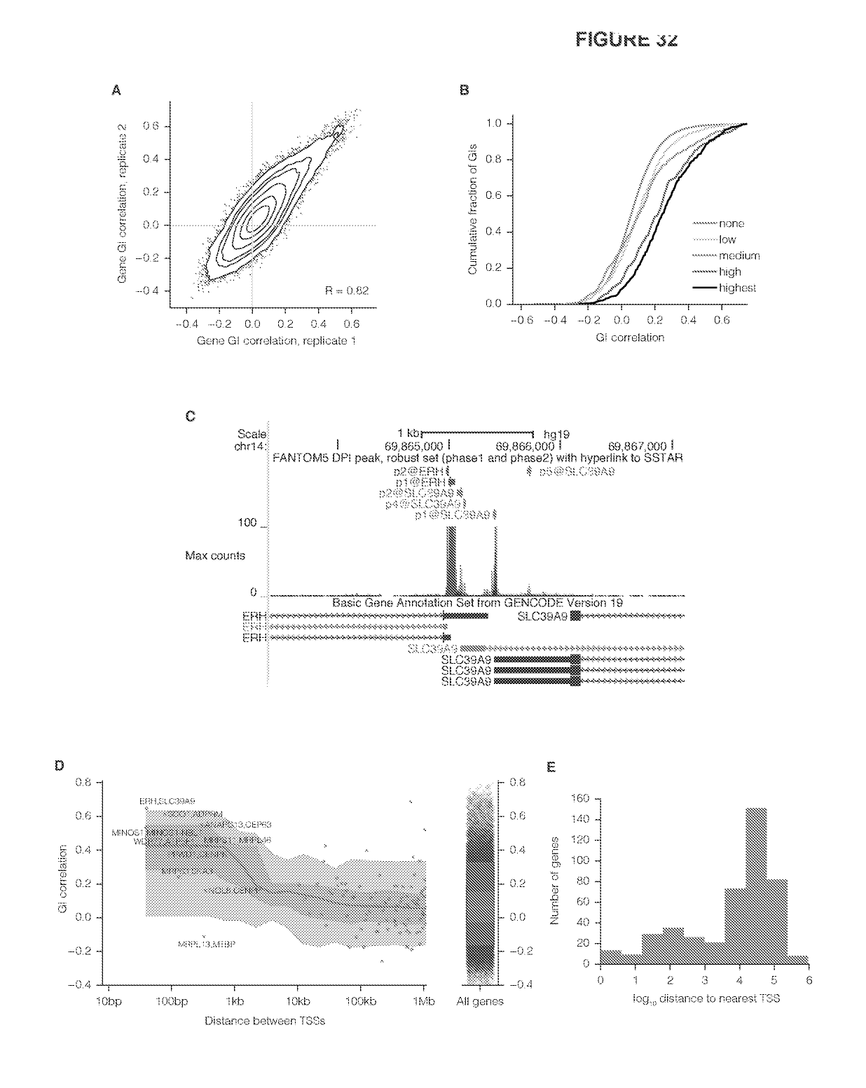

To more systematically explore the ability of correlations to uncover new functional relationships, we next analyzed the distribution of GI profile correlations (FIGS. 31A and 32A). The large majority of genes pairs showed poor correlation, as would be expected; however, many gene pair correlations were stronger than any gene-negative control correlation, suggesting enrichment for functional relationships. Gene correlations within a given cellular compartment were enriched for strong correlations compared to the total distribution or to cross-compartment relationships (FIG. 31B), and highly correlated gene pairs were enriched for known physical interactions annotated by the STRING physical interaction database (FIGS. 31C and 32B) (Szklarczyk et al., 2017). Notably, many highly correlated genes were not captured by STRING annotation, suggesting both unidentified physical interactions and functionally related genes that do not physically interact (FIG. 31C).

To further explore the ability our GI map to identify known and previously unannotated interactions, we examined all gene pairs with a correlation above 0.6. Within this set of highly correlated genes, we found a strong enrichment for genes that encode physical protein complexes annotated by STRING (grey lines, FIG. 31D). We also observed highly correlated gene pairs that were not annotated to interact but were both annotated as important for mitochondrial function in MitoCarta (purple lines, FIG. 31D) (Calvo et al., 2016). A large fraction of these interactions were between mitochondrial ribosome subunits that have been difficult to annotate by mass spectrometry and other physical interaction profiling methods but are annotated by substantial experimental evidence and therefore included in MitoCarta. Even after considering STRING and MitoCarta pairs as known interactions, we also identified a number of unannotated interactions (red lines, FIG. 31D).

Prominent examples of unannotated interactions are identified by our GI map even for well-studied biological processes such as DNA synthesis, ER protein trafficking, the electron transport chain and mitochondrial protein translation. For example, we identify a strong GI correlation between POLE2 and PRIM2 as well as between POLD and CACTIN, two gene pairs required for DNA replication (FIG. 31D). We also find strong correlations between TMEM261 and four NDUF genes present in the GI map (FIG. 31D-E). This mitochondrial cluster provides the first evidence that TMEM261 is a functionally critical subunit of Complex 1 of the electron transport chain despite not being annotated in STRING or MitoCarta. Our finding complements a very recent study showing that TMEM261 is physically associated with Complex 1 (Stroud et al., 2016). Lastly, we identify a strong correlation between the genes ASNA1/CAMLG. ASNA1/CAMLG are functionally homologous to the yeast genes GET3/2 as yeast complementation assays show that the human genes can complement their function; we provide unbiased in vivo support that these genes coordinate protein import to the ER (FIG. 31F). Two gene pairs with unannotated interactions have closely neighboring gene transcription start sites (TSS). We and others have shown CRISPRi can repress expression of TSSs within ˜1 kb of the target site, so in these cases orthogonal methods are required to separate the contribution of each gene perturbation to the GI profile (FIG. 31D starred pairs, FIG. 32C-E). We have annotated all gene pairs within the map that could have a neighbor effect that convolutes analysis (FIG. 32D-E).

Anti-Correlated GIs Reveal Orthogonal Biological Processes.

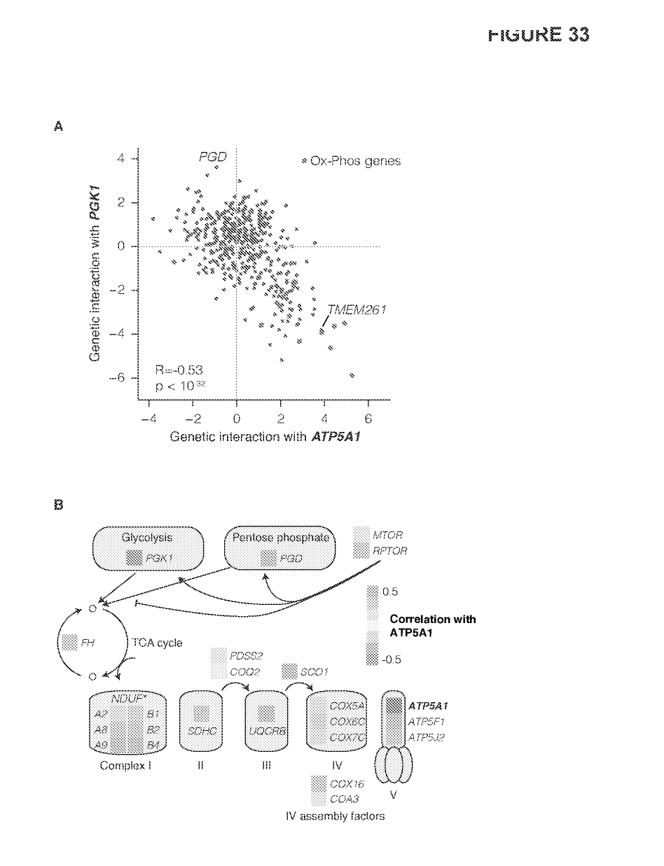

We observed that a number of genes in our map were strongly anti-correlated. Two examples in yeast and human cells, have suggested anti-correlated GIs can reveal genes with related but opposing cellular roles (Bassik et al., 2013; Breslow et al., 2010). To determine the biological basis of anti-correlated GIs in our map, we inspected highly anti-correlated gene pairs and observed a strong anti-correlation between genes required for glycolysis and genes required for oxidative phosphorylation. As one example, we found that PGK1 was strongly anti-correlated with ATP5A1. Repression of ATP5A1 is buffering with repression of genes required for oxidative phosphorylation (OX-PHOS) as well as with other mitochondrial genes, while repression of PGK1 results in a synergistic phenotype with the same genes (FIG. 33A). Broadly, genes upstream of the TCA cycle were anti-correlated with ATP5A1, while genes downstream of the TCA cycle were correlated with ATP5A1 (FIG. 33B). This observation may support the hypothesis that chronic myeloid leukemia cells retain the ability to switch between sugars, lipids and amino acids as a carbon source for ATP production using glycolysis and OX-PHOS metabolism (Hsu and Sabatini, 2008). Anti-correlated gene sets may reveal dynamic and regulated biological processes that act in an orthogonal manner in human cells.

The Structure of GIs in Human Cells

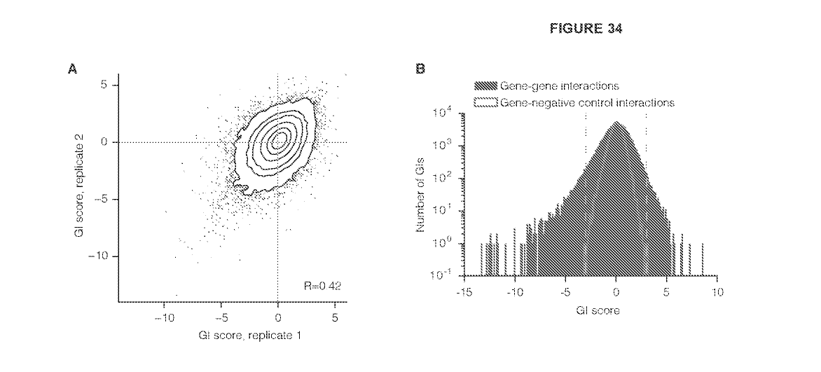

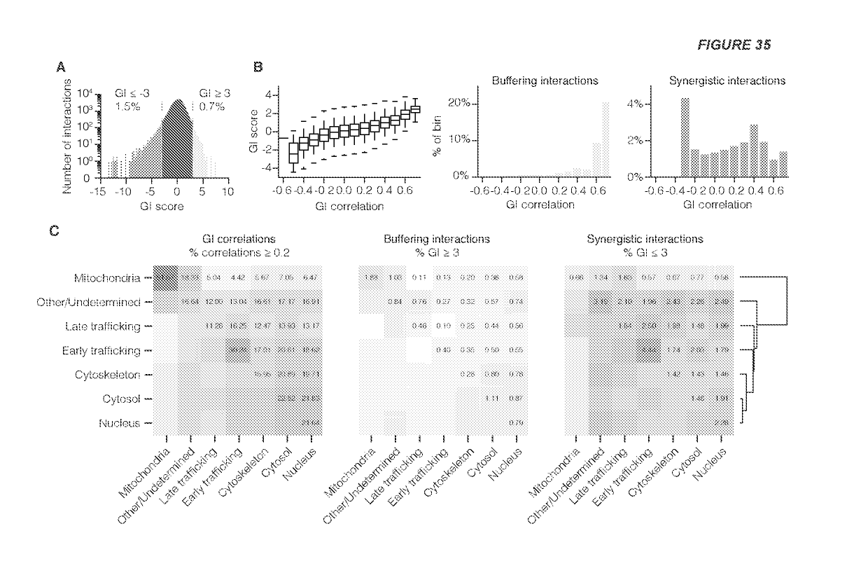

Finally, we investigated the overall structure of GIs in our map. In our map, individual gene-level interactions correlate well and particularly strong interactions are highly reproducible across independent replicates (FIG. 34A). We analyzed the distribution of gene-negative control interactions in our dataset and saw essentially no interactions stronger than +/−3 supporting our confidence in strong gene-pair interactions (FIG. 34B). By this conservative definition, strong GIs are rare, representing just 2.2% of total gene-gene interactions measured (FIG. 35A). The frequency of strong GIs in human cells is similar to the frequency observed in yeast (Costanzo et al., 2010; Schuldiner et al., 2005), although we note that our gene set is enriched for genes required for cell growth and therefore may not reflect an average frequency of GIs across all genes. We observed that strong buffering interactions are most frequent between genes with highly correlated GI profiles (FIG. 35B), as might be expected as buffering GIs are thought to generally identify highly related genes within a protein complex, pathway process or organelle. Interestingly, in this dataset the synergistic interactions were found across all levels of GI correlation as was similarly observed in yeast (FIG. 35B) (Collins et al., 2006). The relationship between gene pair correlations and the frequency of strong SSL and buffering GIs in our experiments resembles the structure of GIs in yeast maps (Collins et al., 2006). The paucity of strong interactions in our data motivates construction of very large GI maps.

Similarly, GI correlations between gene pairs are highest within specific physical compartments of a cell (FIG. 35C). Strong buffering interactions mirror this distribution and are enriched within cell compartments while for SSL interactions there is less enrichment for interactions within subcellular compartments. Instead, we see the majority of SSL interactions occur across subcellular compartments (FIG. 35C).

Comparative GI Mapping of Human Cells Reveals Conservation and Functional Rewiring of GIs.

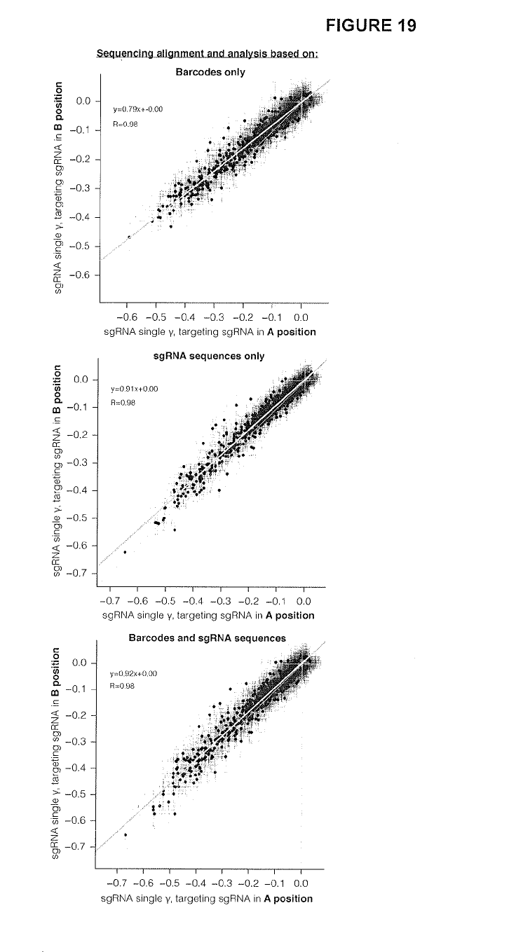

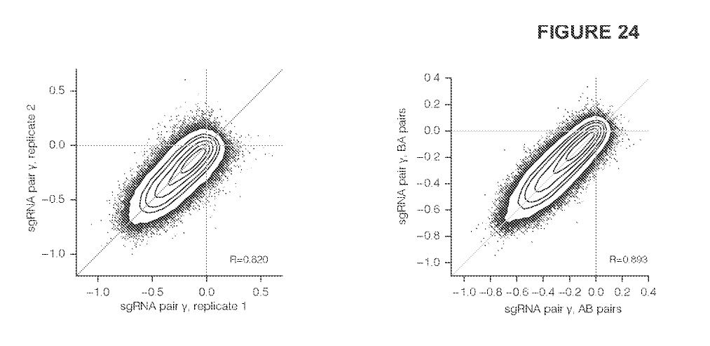

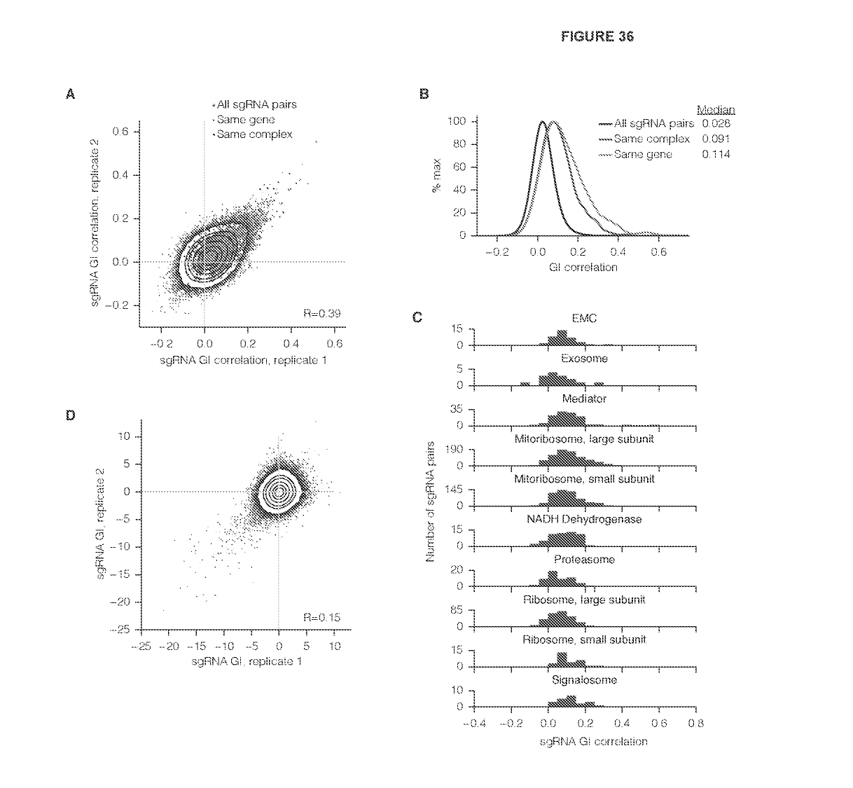

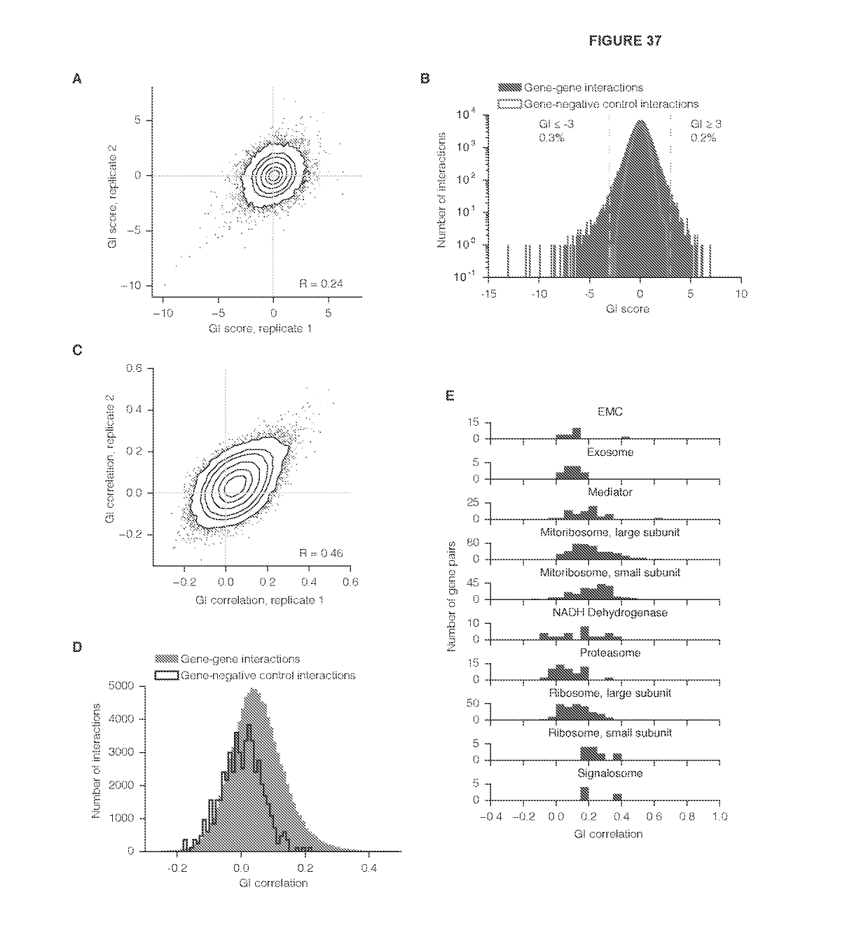

To test whether our GI platform reveals genetic rewiring and GI conservation across distinct human cell types, we screened our GI map library in a second human hematopoietic cancer cell line, namely Jurkat cells, which are a T-cell acute lymphoblastic leukemia cell line. We conducted and analyzed the GI map screens as before in K562 cells to measure GIs in Jurkat cells (FIGS. 19, 20, 24, 36 and 37). Overall, GI correlations between gene pairs are well correlated between the two maps, and intra-complex correlations are enriched for high correlation. GI scores were generally less well correlated. We validated examples of shared strong buffering and synergistic interactions. We also observed and validated functional repurposing of specific gene products between K562 and Jurkat demonstrating rewiring of GIs between human leukemias.

Here we present a combined experimental and analytic framework for high precision ultra-rich GI mapping. We apply this platform to create two high-content large-scale GI maps of functionally and spatially diverse human genes each targeting 222,784 gene pairs. Our maps serve as a broad resource, and our experimental and analytic platform will enable future GI mapping efforts. Analysis of these GI maps supports three main conclusions.

First, we establish the mammalian GI maps as a powerful tool for the unbiased functional characterization of highly diverse genes. The GI signature of a gene yields a high-resolution phenotype enabling one to robustly cluster genes of known biological function and assign function to poorly characterized genes. Specifically, our GI map revealed at least 37 distinct functional gene clusters spanning diverse biological processes such as mitochondrial protein translation, electron transport, ER/Golgi protein trafficking, kinetochore and centromere biology and DNA replication. Many of the functional inferences from GI signatures in our map are novel, establishing the ability of this approach to reveal new biology not anticipated by other methods. In support of this observation, we show that most gene pair GI correlations are conserved between two related hematopoietic cancer cell types. However, we highlight in our comparative GI map analysis that specific genes are functionally repurposed, illustrating the value of mapping diverse human cell types.

Second, we establish the ability to identify unexpected SSL and buffering gene pairs and to link diverse processes and dissect complex pathways. A striking example of an unexpected SSL link between disparate processes was the ability of systematic epistasis analysis to implicate a specific intermediate in cholesterol biosynthesis (IPP) as a DNA damaging agent leading to exquisite dependence on an intact DNA damage response. Similarly, strong buffering interactions between the PAF1 complex, which coordinates Polymerase II transcription, and multiple mitochondrial defects point to unanticipated functional connections between nuclear transcription and mitochondrial bioenergetics. In addition to their importance in illuminating normal biology, SSL and buffering interactions have important implications for the design of therapeutic strategies. For example, genetic suppressors of loss-of-function perturbations can guide development of therapeutic strategies for recessive loss-of-function diseases, and identification of SSL pairs can inform the design of combination therapies.

Third, at a broader level our data begins to shed light on the nature and frequency of GIs in human cells. Strong buffering and SSL interactions are rare and this scarcity illustrates the need for large-scale, systematic and robust methods such as GI mapping capable of identifying and characterizing interacting gene pairs. Buffering interactions occur most frequently between genes of related function and robustly classify known and new components of protein complexes and pathways in our map. Intriguingly, SSL gene interactions are often observed between biological processes that are not correlated in our map or that otherwise lack an obvious functional connection. Expanding the analysis of GI frequency to more genes and cell types will provide insight into polygenic diseases as well as the role of GIs in contributing to missing inheritance seen in association studies.

Our work provides a robust platform for future GI mapping efforts that will complement the rich insights obtained from recent large-scale efforts that use comparative genome-scale CRISPR or RNAi screens across many human cancer cell lines to define gene function (Hart et al., 2015; Tsherniak et al., 2017; Wang et al., 2017). Such approaches reveal cancer genetic dependencies resulting from gain- and loss-of-function mutations and genome copy number alterations. GI maps extend this intellectual framework as they are by definition created experimentally, enabling them to reveal interactions between genetic perturbations which may not be represented in naturally occurring cancer-associated genome variations. Beyond the cancer genome, we envision applying CRISPR cutting, CRISPRi and CRISPRa to model disease-associated genomic variants predicted by genome-wide association studies, transcriptional profiling, epigenetic profiling or DNA sequencing efforts and then using GI maps to dissect specific disease states with high resolution.

Towards a Complete GI Map of the Human Cell

Despite the enormous potential of complete GI maps of human cells, such efforts face several obstacles. A central challenge in creating complete GI maps of human cells is the enormous number of possible gene pairs encoded by the genome. In practice, a complete loss-of-function GI map only need to target genes that are expressed. Most human cell types express 8000-12,000 genes and so a complete loss-of-function GI map of all transcribed genes will require measurement of 40-70 million unique gene pair interactions. A second and related challenge is that the human body is composed of many cell types. We have shown our CRISPRi platform is readily adaptable to many other cell types including induced pluripotent stem cells, which can be differentiated to model various types of human cells including neurons and cardiomyocytes. It will be highly advantageous to minimize the experimental scale required to construct a complete GI map.

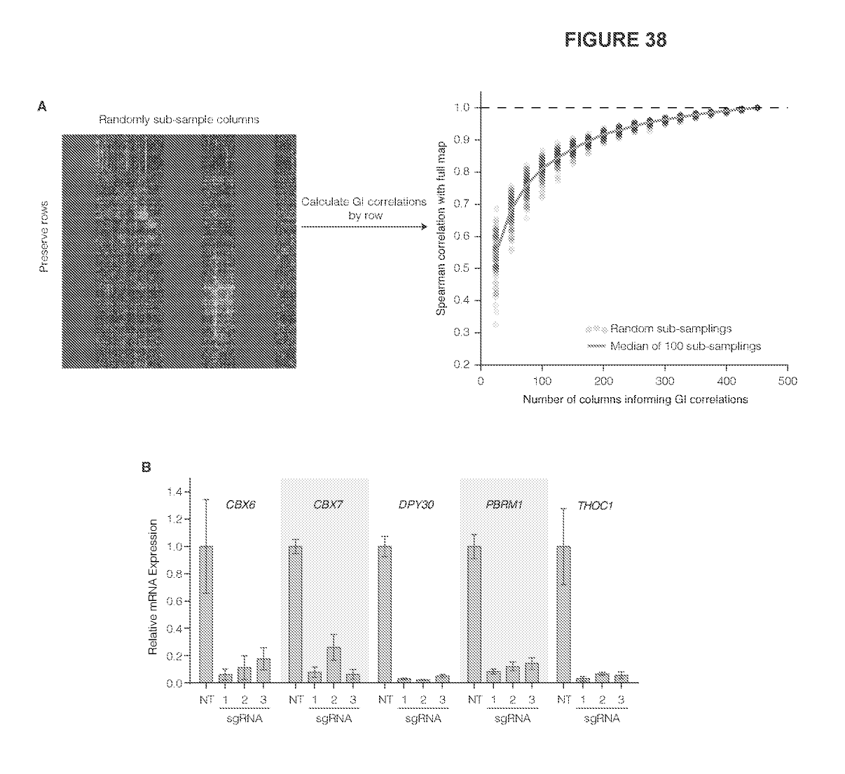

Several considerations suggest it may be possible to greatly reduce the number of measurements while still capturing much of the functional clustering information contained in a complete all-gene by all-gene GI map. One approach would be to construct GI maps with a small set of highly informative query genes that enable one to infer GIs for a large number of functionally similar genes (e.g., picking a single gene to represent a group of genes with highly correlated GIs, such as the mitochondrial ribosome). Selection of such query genes could be aided by first generating complete GI maps for several robust cell models to define an optimally informative gene set. An alternative strategy would be to simply randomly select a query gene set and thus avoid biases introduced by gene selection. We simulated this latter approach by randomly sub-sampling columns from the GI map and re-calculating GI correlations based on rows. Even with fewer than half the columns, row-row correlations have over 0.9 Spearman correlation with the full map, suggesting that the hierarchy and functional clustering can be preserved even with a random set of query genes (FIG. 38A). We anticipate that any enrichment for more informative query genes, guided by gene annotation or external datasets, will further maximize the information content/cost trade-off

A distinct challenge for generating comprehensive GI maps is the need to have a set of highly active sgRNAs against all genes. In the present GI map, we used previously validated sgRNAs, which allows us to reliably measure the GI signature for genes using just 1-3 perturbations. There has been considerable progress in design algorithms for CRISPR nuclease, CRISPRa, and CRISPRi which will greatly facilitate GI mapping efforts. Indeed, even focusing on genes that had proven difficult to knockdown using an algorithm employed in an earlier mapping effort (Du et al., 2017), we find that 2 of every 3 sgRNAs predicted to be highly active by our current algorithm give greater than 90% repression of the target gene, and all give greater than 75% repression (FIG. 38B). This is consistent with previous results measuring CRISPRi knockdown of lncRNAs selected in an unbiased manner (Liu et al., 2017).

In summary, the tools, techniques and analytical framework now exist to systematically map the genetic landscape of mammalian cells. Given the rich information from the present map, such efforts will be transformative for the study of normal biology and pathological states.

### REFERENCES

Adamson, B., Norman, T. M., Jost, M., Cho, M. Y., Nuñez, J. K., Chen, Y., Villalta, J. E., Gilbert, L. A., Horlbeck, M. A., Hein, M. Y., et al. (2016). A Multiplexed Single-Cell CRISPR Screening Platform Enables Systematic Dissection of the Unfolded Protein Response. Cell 167, 1867-1882.e21.

Acosta-Alvear, D., Zhou, Y., Blais, A., Tsikitis, M., Lents, N. H., Arias, C., Lennon, C. J., Kluger, Y., and Dynlacht, B. D. (2007). XBP1 controls diverse cell type- and condition-specific transcriptional regulatory networks. Mol. Cell 27, 53-66.

Aguirre, A. J., Meyers, R. M., Weir, B. A., Vazquez, F., Zhang, C.-Z., Ben-David, U., Cook, A., Ha, G., Harrington, W. F., Doshi, M. B., et al. (2016). Genomic Copy Number Dictates a Gene-Independent Cell Response to CRISPR/Cas9 Targeting. Cancer Discov. 6, 914-929.

Babu, M., Arnold, R., Bundalovic-Torma, C., Gagarinova, A., Wong, K. S., Kumar, A., Stewart, G., Samanfar, B., Aoki, H., Wagih, O., et al. (2014). Quantitative genome-wide genetic interaction screens reveal global epistatic relationships of protein complexes in Escherichia coli. PLoS Genet. 10, e1004120.

Boettcher, M., and McManus, M. T. (2015). Choosing the right tool for the job: RNAi, TALEN, or CRISPR. Mol. Cell 58, 575-585.

Bandyopadhyay, S., Mehta, M., Kuo, D., Sung, M.-K., Chuang, R., Jaehnig, E. J., Bodenmiller, B., Licon, K., Copeland, W., Shales, M., et al. (2010). Rewiring of genetic networks in response to DNA damage. Science 330, 1385-1389.

Bassik, M. C., Kampmann, M., Lebbink, R. J., Wang, S., Hein, M.Y., Poser, I., Weibezahn, J., Horlbeck, M. A., Chen, S., Mann, M., et al. (2013). A systematic mammalian genetic interaction map reveals pathways underlying ricin susceptibility. Cell 152, 909-922.

Battle, A., Jonikas, M. C., Walter, P., Weissman, J. S., and Koller, D. (2010). Automated identification of pathways from quantitative genetic interaction data. Mol. Syst. Biol. 6, 379.

Boyle, E. A., Li, Y. I., and Pritchard, J. K. (2017). An Expanded View of Complex Traits: From Polygenic to Omnigenic. Cell 169, 1177-1186.

Breslow, D. K., Collins, S. R., Bodenmiller, B., Aebersold, R., Simons, K., Shevchenko, A., Ejsing, C. S., and Weissman, J. S. (2010). Orm family proteins mediate sphingolipid homeostasis. Nature 463, 1048-1053.

Calvo, S. E., Clauser, K. R., and Mootha, V. K. (2016). MitoCarta2.0: an updated inventory of mammalian mitochondrial proteins. Nucleic Acids Res. 44, D1251-1257.

Candès, E. J., Li, X., Ma, Y., and Wright, J. (2011). Robust principal component analysis? Journal of the ACM (JACM) 58, 11.

Carbon, S., Ireland, A., Mungall, C. J., Shu, S., Marshall, B., and Lewis, S. (2009). AmiGO: online access to ontology and annotation data. Bioinformatics 25, 288-289.

Collins, S. R., Schuldiner, M., Krogan, N. J., and Weissman, J. S. (2006). A strategy for extracting and analyzing large-scale quantitative epistatic interaction data. Genome Biol. 7, R63.

Collins, S. R., Miller, K. M., Maas, N. L., Roguev, A., Fillingham, J., Chu, C. S., Schuldiner, M., Gebbia, M., Recht, J., Shales, M., et al. (2007). Functional dissection of protein complexes involved in yeast chromosome biology using a genetic interaction map. Nature 446, 806-810.

Costanzo, M., Baryshnikova, A., Bellay, J., Kim, Y., Spear, E. D., Sevier, C. S., Ding, H., Koh, J. L. Y., Toufighi, K., Mostafavi, S., et al. (2010). The genetic landscape of a cell. Science 327, 425-431.

Costanzo, M., VanderSluis, B., Koch, E. N., Baryshnikova, A., Pons, C., Tan, G., Wang, W., Usaj, M., Hanchard, J., Lee, S. D., et al. (2016). A global genetic interaction network maps a wiring diagram of cellular function. Science 353.

Du, D., Roguev, A., Gordon, D. E., Chen, M., Chen, S.-H., Shales, M., Shen, J. P., Ideker, T., Mali, P., Qi, L. S., et al. (2017). Genetic interaction mapping in mammalian cells using CRISPR interference. Nat. Methods 14, 577-580.

Elena, S. F., and Lenski, R. E. (1997). Test of synergistic interactions among deleterious mutations in bacteria. Nature 390, 395-398.

Fischer, B., Sandmann, T., Horn, T., Billmann, M., Chaudhary, V., Huber, W., and Boutros, M. (2015). A map of directional genetic interactions in a metazoan cell. eLife 4.

Friedman, J., Hastie, T., and Tibshirani, R. (2001). The elements of statistical learning Springer series in statistics Springer, Berlin).

Frost, A., Elgort, M.G., Brandman, O., Ives, C., Collins, S. R., Miller-Vedam, L., Weibezahn, J., Hein, M. Y., Poser, I., Mann, M., et al. (2012). Functional repurposing revealed by comparing S. pombe and S. cerevisiae genetic interactions. Cell 149, 1339-1352.

Gilbert, L. A., Horlbeck, M. A., Adamson, B., Villalta, J. E., Chen, Y., Whitehead, E. H., Guimaraes, C., Panning, B., Ploegh, H. L., and Bassik, M. C. (2014). Genome-scale CRISPR-mediated control of gene repression and activation. Cell 159, 647-661.

Gilbert, L. A., Larson, M. H., Morsut, L., Liu, Z., Brar, G. A., Torres, S. E., Stern-Ginossar, N., Brandman, O., Whitehead, E. H., Doudna, J. A., et al. (2013). CRISPR-mediated modular RNA-guided regulation of transcription in eukaryotes. Cell 154, 442-451.

Gray, A. N., Koo, B.-M., Shiver, A. L., Peters, J. M., Osadnik, H., and Gross, C. A. (2015). High-throughput bacterial functional genomics in the sequencing era. Curr. Opin. Microbiol. 27, 86-95.

Guénolé, A., Srivas, R., Vreeken, K., Wang, Z. Z., Wang, S., Krogan, N. J., Ideker, T., and van Attikum, H. (2013). Dissection of DNA damage responses using multiconditional genetic interaction maps. Mol. Cell 49, 346-358.

Gu, W., Crawford, E. D., O'Donovan, B. D., Wilson, M. R., Chow, E. D., Retallack, H., and DeRisi, J. L. (2016). Depletion of Abundant Sequences by Hybridization (DASH): using Cas9 to remove unwanted high-abundance species in sequencing libraries and molecular counting applications. Genome Biol. 17, 41.

Haldimann, A., and Wanner, B. L. (2001). Conditional-replication, integration, excision, and retrieval plasmid-host systems for gene structure-function studies of bacteria. J. Bacteriol. 183, 6384-6393.

Hamanaka, R. B., Bennett, B. S., Cullinan, S. B., and Diehl, J. A. (2005). PERK and GCN2 Contribute to eIF2α Phosphorylation and Cell Cycle Arrest after Activation of the Unfolded Protein Response Pathway. Mol. Biol. Cell 16, 5493-5501.

Han, J., Back, S. H., Hur, J., Lin, Y., Gildersleeve, R., Shan, J., Yuan, C. L., Krokowski, D., Wang, S., Hatzoglou, M., et al. (2013). ER-stress-induced transcriptional regulation increases protein synthesis leading to cell death. Nat. Cell Biol. 15, 481-490.

Han, K., Jeng, E. E., Hess, G. T., Morgens, D. W., Li, A., and Bassik, M. C. (2017). Synergistic drug combinations for cancer identified in a CRISPR screen for pairwise genetic interactions. Nat. Biotechnol. 35, 463-474.

Heimberg, G., Bhatnagar, R., El-Samad, H., and Thomson, M. (2016). Low Dimensionality in Gene Expression Data Enables the Accurate Extraction of Transcriptional Programs from Shallow Sequencing. Cell Syst 2, 239-250.

Hart, T., Chandrashekhar, M., Aregger, M., Steinhart, Z., Brown, K. R., MacLeod, G., Mis, M., Zimmermann, M., Fradet-Turcotte, A., Sun, S., et al. (2015). High-Resolution CRISPR Screens Reveal Fitness Genes and Genotype-Specific Cancer Liabilities. Cell 163, 1515-1526.

de Hoon, M. J. L., Imoto, S., Nolan, J., and Miyano, S. (2004). Open source clustering software. Bioinforma. Oxf. Engl. 20, 1453-1454.

Horlbeck, M. A., Gilbert, L. A., Villalta, J. E., Adamson, B., Pak, R. A., Chen, Y., Fields, A. P., Park, C. Y., Corn, J. E., and Kampmann, M. (2016a). Compact and highly active next-generation libraries for CRISPR-mediated gene repression and activation. eLife 5, e19760.

Horlbeck, M. A., Witkowsky, L. B., Guglielmi, B., Replogle, J. M., Gilbert, L. A., Villalta, J. E., Torigoe, S. E., Tjian, R., and Weissman, J. S. (2016b). Nucleosomes impede Cas9 access to DNA in vivo and in vitro. eLife 5.

Hsu, P. P., and Sabatini, D. M. (2008). Cancer cell metabolism: Warburg and beyond. Cell 134, 703-707.

Huang, D. W., Sherman, B. T., and Lempicki, R. A. (2009). Systematic and integrative analysis of large gene lists using DAVID bioinformatics resources. Nat Protoc 4, 44-57.

Itzhak, D. N., Tyanova, S., Cox, J., and Bomer, G. H. (2016). Global, quantitative and dynamic mapping of protein subcellular localization. eLife 5.

Jaitin, D. A., Kenigsberg, E., Keren-Shaul, H., Elefant, N., Paul, F., Zaretsky, I., Mildner, A., Cohen, N., Jung, S., Tanay, A., and Amit, I. (2014). Massively parallel single-cell RNA-seq for marker-free decomposition of tissues into cell types. Science 343, 776-779.

Jonikas, M. C., Collins, S. R., Denic, V., Oh, E., Quan, E. M., Schmid, V., Weibezahn, J., Schwappach, B., Walter, P., Weissman, J. S., and Schuldiner, M. (2009). Comprehensive characterization of genes required for protein folding in the endoplasmic reticulum. Science 323, 1693-1697.

Kabadi, A. M., Ousterout, D. G., Hilton, I. B., and Gersbach, C. A. (2014). Multiplex CRISPR/Cas9-based genome engineering from a single lentiviral vector. Nucleic Acids Res. 42, e147.

Kampmann, M., Bassik, M. C., and Weissman, J. S. (2013). Integrated platform for genome-wide screening and construction of high-density genetic interaction maps in mammalian cells. Proc. Natl. Acad. Sci. U.S.A. 110, E2317-2326.

Kampmann, M., Bassik, M. C., and Weissman, J. S. (2014). Functional genomics platform for pooled screening and generation of mammalian genetic interaction maps. Nat Protoc 9, 1825-1847.

Kanda, S., Yanagitani, K., Yokota, Y., Esaki, Y., and Kohno, K. (2016). Autonomous translational pausing is required for XBP1u mRNA recruitment to the ER via the SRP pathway. Proc. Natl. Acad. Sci. U.S.A. 113, E5895.

Klein, A. M., Mazutis, L., Akartuna, I., Tallapragada, N., Veres, A., Li, V., Peshkin, L., Weitz, D. A., and Kirschner, M. W. (2015). Droplet barcoding for single-cell transcriptomics applied to embryonic stem cells. Cell 161, 1187-1201.

Komor, A. C., Kim, Y. B., Packer, M. S., Zuris, J. A., and Liu, D. R. (2016). Programmable editing of a target base in genomic DNA without double-stranded DNA cleavage. Nature 533, 420-424.

Kramer, M. H., Farré, J.-C., Mitra, K., Yu, M.K., Ono, K., Demchak, B., Licon, K., Flagg, M., Balakrishnan, R., Cherry, J. M., et al. (2017). Active Interaction Mapping Reveals the Hierarchical Organization of Autophagy. Mol. Cell 65, 761-774.e5.

Kuleshov, M. V., Jones, M. R., Rouillard, A. D., Fernandez, N. F., Duan, Q., Wang, Z., Koplev, S., Jenkins, S. L., Jagodnik, K. M., Lachmann, A., et al. (2016). Enrichr: a comprehensive gene set enrichment analysis web server 2016 update. Nucleic Acids Res. 44, 90.

Langmead et al. (2009). Ultrafast and memory-efficient alignment of short DNA sequences to the human genome. Genome Biology. 10:R25.

Lee, A., Iwakoshi, N. N., and Glimcher, L. H. (2003). XBP-1 regulates a subset of endoplasmic reticulum resident chaperone genes in the unfolded protein response. Mol. Cell. Biol. 23, 7448-7459.

Liang, S., Zhang, W., McGrath, B. C., Zhang, P., and Cavener, D. R. (2006). PERK (eIF2alpha kinase) is required to activate the stress-activated MAPKs and induce the expression of immediate-early genes upon disruption of ER calcium homoeostasis. Biochem. J. 393, 201-209.

Lin, J. H., Li, H., Yasumura, D., Cohen, H. R., Zhang, C., Panning, B., Shokat, K. M., Lavail, M. M., and Walter, P. (2007). IRE1 signaling affects cell fate during the unfolded protein response. Science 318, 944-949.

Lin, Z., Chen, M., and Ma, Y. (2010). The augmented lagrange multiplier method for exact recovery of corrupted low-rank matrices. arXiv Preprint arXiv:1009.5055.

Liu, S. J., Horlbeck, M. A., Cho, S. W., Birk, H. S., Malatesta, M., He, D., Attenello, F. J., Villalta, J. E., Cho, M. Y., Chen, Y., et al. (2017). CRISPRi-based genome-scale identification of functional long noncoding RNA loci in human cells. Science 355.

Macosko, E. Z., Basu, A., Satija, R., Nemesh, J., Shekhar, K., Goldman, M., Tirosh, I., Bialas, A. R., Kamitaki, N., Martersteck, E. M., et al. (2015). Highly Parallel Genome-wide Expression Profiling of Individual Cells Using Nanoliter Droplets. Cell 161, 1202-1214.

Mandegar, M. A., Huebsch, N., Frolov, E. B., Shin, E., Truong, A., Olvera, M. P., Chan, A. H., Miyaoka, Y., Holmes, K., Spencer, C. I., et al. (2016). CRISPR Interference Efficiently Induces Specific and Reversible Gene Silencing in Human iPSCs. Cell Stem Cell 18, 541-553.

Manolio, T. A., Collins, F. S., Cox, N. J., Goldstein, D. B., Hindorff, L. A., Hunter, D. J., McCarthy, M. I., Ramos, E. M., Cardon, L. R., Chakravarti, A., et al. (2009). Finding the missing heritability of complex diseases. Nature 461, 747-753.

Müller-Kuller, U., Ackermann, M., Kolodziej, S., Brendel, C., Fritsch, J., Lachmann, N., Kunkel, H., Lausen, J., Schambach, A., Moritz, T., and Grez, M. (2015). A minimal ubiquitous chromatin opening element (UCOE) effectively prevents silencing of juxtaposed heterologous promoters by epigenetic remodeling in multipotent and pluripotent stem cells. Nucl. Acids Res. gkv019.

Munoz, D. M., Cassiani, P. J., Li, L., Billy, E., Korn, J. M., Jones, M. D., Golji, J., Ruddy, D. A., Yu, K., McAllister, G., et al. (2016). CRISPR Screens Provide a Comprehensive Assessment of Cancer Vulnerabilities but Generate False-Positive Hits for Highly Amplified Genomic Regions. Cancer Discov. 6, 900-913.

Nishimasu, H., Ran, F.A., Hsu, P. D., Konermann, S., Shehata, S.I., Dohmae, N., Ishitani, R., Zhang, F., and Nureki, O. (2014). Crystal structure of Cas9 in complex with guide RNA and target DNA. Cell 156, 935-949.

Nissim, L., Perli, S. D., Fridkin, A., Perez-Pinera, P., and Lu, T. K. (2014). Multiplexed and programmable regulation of gene networks with an integrated RNA and CRISPR/Cas toolkit in human cells. Mol. Cell 54, 698-710.

Oslowski, C. M., and Urano, F. (2011). Measuring ER stress and the unfolded protein response using mammalian tissue culture system. Meth. Enzymol. 490, 71.

Pan, X., Yuan, D. S., Xiang, D., Wang, X., Sookhai-Mahadeo, S., Bader, J. S., Hieter, P., Spencer, F., and Boeke, J. D. (2004). A robust toolkit for functional profiling of the yeast genome. Mol. Cell 16, 487-496.

Pan, X., Ye, P., Yuan, D. S., Wang, X., Bader, J. S., and Boeke, J. D. (2006). A DNA integrity network in the yeast Saccharomyces cerevisiae. Cell 124, 1069-1081.

Plumb, R., Zhang, Z., Appathurai, S., and Mariappan, M. (2015). A functional link between the co-translational protein translocation pathway and the UPR. Elife 4,

Qi, L. S., Larson, M. H., Gilbert, L. A., Doudna, J. A., Weissman, J. S., Arkin, A. P., and Lim, W. A. (2013). Repurposing CRISPR as an RNA-guided platform for sequence-specific control of gene expression. Cell 152, 1173-1183.

Roguev, A., Wiren, M., Weissman, J. S., and Krogan, N. J. (2007). High-throughput genetic interaction mapping in the fission yeast Schizosaccharomyces pombe. Nat. Methods 4, 861-866.

Roguev, A., Talbot, D., Negri, G. L., Shales, M., Cagney, G., Bandyopadhyay, S., Panning, B., and Krogan, N. J. (2013). Quantitative genetic-interaction mapping in mammalian cells. Nat. Methods 10, 432-437.

Rosenbluh, J., Mercer, J., Shrestha, Y., Oliver, R., Tamayo, P., Doench, J.G., Tirosh, I., Piccioni, F., Hartenian, E., Horn, H., et al. (2016). Genetic and Proteomic Interrogation of Lower Confidence Candidate Genes Reveals Signaling Networks in β-Catenin-Active Cancers. Cell Syst. 3, 302-316.e4.

Sack, L. M., Davoli, T., Xu, Q., Li, M. Z., and Elledge, S. J. (2016). Sources of Error in Mammalian Genetic Screens. G3 (Bethesda) 6,2781-2790.

Saldanha, A. J. (2004). Java Treeview—extensible visualization of microarray data. Bioinforma. Oxf. Engl. 20, 3246-3248.

Schuldiner, M., Collins, S. R., Thompson, N. J., Denic, V., Bhamidipati, A., Punna, T., Ihmels, J., Andrews, B., Boone, C., Greenblatt, J. F., et al. (2005). Exploration of the function and organization of the yeast early secretory pathway through an epistatic miniarray profile. Cell 123,507-519.

Segrè, D., Deluna, A., Church, G. M., and Kishony, R. (2005). Modular epistasis in yeast metabolism. Nat. Genet. 37,77-83.

Shalem, O., Sanjana, N. E., and Zhang, F. (2015). High-throughput functional genomics using CRISPR-Cas9. Nat. Rev. Genet. 16,299-311.

Shen, J. P., Zhao, D., Sasik, R., Luebeck, J., Birmingham, A., Bojorquez-Gomez, A., Licon, K., Klepper, K., Pekin, D., Beckett, A. N., et al. (2017). Combinatorial CRISPR-Cas9 screens for de novo mapping of genetic interactions. Nat. Methods 14, 573-576.

Shi, J., Wang, E., Milazzo, J. P., Wang, Z., Kinney, J. B., and Vakoc, C. R. (2015). Discovery of cancer drug targets by CRISPR-Cas9 screening of protein domains. Nat. Biotechnol. 33, 661-667.

Shi, Z., Fujii, K., Kovary, K. M., Genuth, N. R., Röst, H. L., Teruel, M. N., and Barna, M. (2017). Heterogeneous Ribosomes Preferentially Translate Distinct Subpools of mRNAs Genome-wide. Mol. Cell 67,71-83.e7.

Shoulders, M. D., Ryno, L. M., Genereux, J. C., Moresco, J. J., Tu, P. G., Wu, C., Yates, J. R., Su, A. I., Kelly, J. W., and Wiseman, R. L. (2013). Stress-independent activation of XBP1s and/or ATF6 reveals three functionally diverse ER proteostasis environments. Cell Rep 3, 1279-1292.

Sidrauski, C., Tsai, J. C., Kampmann, M., Hearn, B. R., Vedantham, P., Jaishankar, P., Sokabe, M., Mendez, A. S., Newton, B. W., Tang, E. L., et al. (2015). Pharmacological dimerization and activation of the exchange factor eIF2B antagonizes the integrated stress response. Elife 4, e07314.

Simsek, D., Tiu, G. C., Flynn, R. A., Byeon, G. W., Leppek, K., Xu, A.F., Chang, H. Y., and Barna, M. (2017). The Mammalian Ribo-interactome Reveals Ribosome Functional Diversity and Heterogeneity. Cell 169, 1051-1065.e18.

Smoot, M. E., Ono, K., Ruscheinski, J., Wang, P.-L., and Ideker, T. (2011). Cytoscape 2.8: new features for data integration and network visualization. Bioinforma. Oxf. Engl. 27, 431-432.

Smyth, R. P., Davenport, M. P., and Mak, J. (2012). The origin of genetic diversity in HIV-1. Virus Res. 169, 415-429.

Stroud, D. A., Surgenor, E. E., Formosa, L. E., Reljic, B., Frazier, A. E., Dibley, M. G., Osellame, L. D., Stait, T., Beilharz, T. H., Thorburn, D. R., et al. (2016). Accessory subunits are integral for assembly and function of human mitochondrial complex I. Nature 538, 123-126.

Szklarczyk, D., Morris, J. H., Cook, H., Kuhn, M., Wyder, S., Simonovic, M., Santos, A., Doncheva, N. T., Roth, A., Bork, P., et al. (2017). The STRING database in 2017: quality-controlled protein-protein association networks, made broadly accessible. Nucleic Acids Res. 45, D362-D368.

Thul, P. J., Åkesson, L., Wiking, M., Mandessian, D., Geladaki, A., Ait Blal, H., Alm, T., Asplund, A., Björk, L., Breckels, L. M., et al. (2017). A subcellular map of the human proteome. Science 356.

Tong, A. H., Evangelista, M., Parsons, A. B., Xu, H., Bader, G. D., Pagé, N., Robinson, M., Raghibizadeh, S., Hogue, C. W., Bussey, H., et al. (2001). Systematic genetic analysis with ordered arrays of yeast deletion mutants. Science 294, 2364-2368.

Tong, A. H. Y., Lesage, G., Bader, G. D., Ding, H., Xu, H., Xin, X., Young, J., Berriz, G. F., Brost, R. L., Chang, M., et al. (2004). Global mapping of the yeast genetic interaction network. Science 303, 808-813.

Tsherniak, A., Vazquez, F., Montgomery, P. G., Weir, B. A., Kryukov, G., Cowley, G. S., Gill, S., Harrington, W. F., Pantel, S., Krill-Burger, J. M., et al. (2017). Defining a Cancer Dependency Map. Cell 170, 564-576.e16.

Van Der Maaten, L. (2014). Accelerating t-SNE using tree-based algorithms. Journal of Machine Learning Research 15, 3221-3245.

Walter, P., and Ron, D. (2011). The unfolded protein response: from stress pathway to homeostatic regulation. Science 334, 1081-1086.

Wang, T., Birsoy, K., Hughes, N. W., Krupczak, K. M., Post, Y., Wei, J. J., Lander, E. S., and Sabatini, D. M. (2015). Identification and characterization of essential genes in the human genome. Science 350, 1096-1101.

Wang, T., Yu, H., Hughes, N. W., Liu, B., Kendirli, A., Klein, K., Chen, W. W., Lander, E. S., and Sabatini, D. M. (2017). Gene Essentiality Profiling Reveals Gene Networks and Synthetic Lethal Interactions with Oncogenic Ras. Cell 168, 890-903.e15.

Wang, Y., Shen, J., Arenzana, N., Tirasophon, W., Kaufman, R. J., and Prywes, R. (2000). Activation of ATF6 and an ATF6 DNA binding site by the endoplasmic reticulum stress response. J. Biol. Chem. 275, 27013-27020.

Wilkin, D. J., Kutsunai, S. Y., and Edwards, P. A. (1990). Isolation and sequence of the human farnesyl pyrophosphate synthetase cDNA. Coordinate regulation of the mRNAs for farnesyl pyrophosphate synthetase, 3-hydroxy-3-methylglutaryl coenzyme A reductase, and 3-hydroxy-3-methylglutaryl coenzyme A synthase by phorbol ester. J. Biol. Chem. 265, 4607-4614.

Wong, A. S. L., Choi, G. C. G., Cui, C. H., Pregernig, G., Milani, P., Adam, M., Perli, S. D., Kazer, S. W., Gaillard, A., Hermann, M., et al. (2016). Multiplexed barcoded CRISPR-Cas9 screening enabled by CombiGEM. Proc. Natl. Acad. Sci. U.S.A. 113, 2544-2549.

Yamamoto, K., Sato, T., Matsui, T., Sato, M., Okada, T., Yoshida, H., Harada, A., and Mori, K. (2007). Transcriptional induction of mammalian ER quality control proteins is mediated by single or combined action of ATF6α and XBP1. Developmental Cell 13, 365-376.

Zheng, G. X. Y., Terry, J. M., Belgrader, P., Ryvkin, P., Bent, Z. W., Wilson, R., Ziraldo, S. B., Wheeler, T. D., McDermott, G. P., Zhu, J., et al. (2016). Massively parallel digital transcriptional profiling of single cells. bioRxiv

1. A nucleic acid construct comprising multiple expression cassettes wherein each expression cassette comprises:
a) a polynucleotide sequence comprising an RNA polymerase III promoter operably linked to a nucleic acid encoding a small guide RNA (sgRNA) comprising a DNA targeting sequence and a constant region that interacts with a site-directed nuclease; and
b) a pair of unique barcode sequences that flank the polynucleotide sequence comprising the RNA polymerase III promoter operably linked to the nucleic acid encoding a small guide RNA (sgRNA),
wherein the RNA polymerase III promoter in each cassette of the nucleic acid construct has a different sequence.

2. The nucleic acid construct of claim 1, wherein the constant region of the sgRNA in each cassette of the nucleic acid construct has a different sequence.

3. The nucleic acid construct of claim 1, wherein the constant region of the sgRNA in each cassette of the nucleic acid construct has an identical sequence.

4. The nucleic acid construct of claim 1, wherein the construct has two expression cassettes.

5. The nucleic acid construct of claim 1, wherein the construct has three expression cassettes.

6. The nucleic acid construct of claim 1, wherein the RNA polymerase III promoters are from different mammalian species.

7. The nucleic acid construct of claim 1, wherein the sgRNA interacts with an enzymatically active site-directed nuclease.

8. The nucleic acid construct of claim 7, wherein the site-directed nuclease is a Cas9 polypeptide.

9. The nucleic acid construct of claim 1, wherein the the sgRNA interacts with a deactivated site-directed nuclease.

10. The nucleic acid construct of claim 9, wherein the site-directed nuclease is a deactivated Cas9 (dCas9) polypeptide.

11. A vector comprising the nucleic acid construct of claim 1.

12. The vector of claim 11, wherein the vector is a lentiviral vector.

13. A method for sequencing a first and a second sgRNA that target a first and a second DNA target in a genome of a cell comprising:
a) infecting a plurality of mammalian cells with a plurality of vectors to form a plurality of vector-infected cells, wherein each vector comprises:
i) a first polynucleotide sequence comprising a first RNA polymerase III promoter operably linked to a nucleic acid encoding a first sgRNA comprising a sequence that targets a first DNA target in the genome and a first constant region that interacts with a site directed nuclease; and a pair of unique barcode sequences that flank the polynucleotide sequence comprising the RNA polymerase III promoter operably linked to the nucleic acid encoding the first sgRNA; and
ii) a second polynucleotide sequence comprising a second RNA polymerase III promoter operably linked to a nucleic acid encoding a second sgRNA comprising a sequence that targets a second DNA target in the genome and a second constant region that interacts with a site directed nuclease; and a pair of unique barcode sequences that flank the polynucleotide sequence comprising the RNA polymerase III promoter operably linked to the nucleic acid encoding the second sgRNA; and
b) expressing a site-directed nuclease in the mammalian cells;
c) separating a selected pool of cells expressing a detectable phenotype from the plurality of infected cells;
d) amplifying DNA comprising the nucleic acid encoding the first sgRNA and the nucleic acid encoding the second sgRNA in each cell with a pair of primers;
wherein optionally, at least one of the primers includes a sample barcode sequence, and wherein the amplified DNA contains two adjacent barcodes flanked by the first and second sgRNAs;
e) sequencing the nucleic acid encoding the first sgRNA and the nucleic acid encoding the second sgRNA in each cell;
f) optionally sequencing the sample barcode;
g) sequencing both adjacent barcode sequences to obtain a barcode sequence combination for each cell;
h) comparing the barcode sequence combination obtained from each cell with the combination of the unique barcode sequence of the first sgRNA and the unique barcode sequence of the second sgRNA in the cell; and
i) identifying the first and the second sgRNA that target a first and a second DNA target in cells comprising a combination of barcode sequences corresponding to the combination of the unique barcode sequence of the first sgRNA and the unique barcode sequence of the second sgRNA in the cell.

14. The method of claim 13, wherein the first sgRNA and the second sgRNA are sequenced on the same strand of amplified DNA.

15. The method of 14, wherein the adjacent barcodes are sequenced on the opposite strand of amplified DNA.

16. The method of claim 12, wherein the vector is a lentiviral vector.

17. The method of claim 13, further comprising infecting the mammalian cells with a vector comprising a polynucleotide sequence encoding the site-directed nuclease prior to or subsequent to infecting the cells with the plurality of vectors.

18. The method of claim 13, wherein the first RNA polymerase III promoter and the second RNA polymerase III promoter have different sequences.

19. The method of claim 18, wherein the first RNA polymerase III promoter and the second RNA polymerase III promoter are from different species.

20. The method of claim 13, wherein the first constant region and the second constant region have different sequences.

21. The method of claim 13, wherein the first constant region and the second constant region have identical sequences.

22. The method of claim 13, wherein the site-directed nuclease is an enzymatically active site-directed nuclease.

23. The method of claim 22, wherein the site-directed nuclease is a Cas9 polypeptide.

24. The method of claim 13, wherein the site-directed nuclease is a deactivated site-directed nuclease.

25. The method of claim 24, wherein the deactivated site-directed nuclease is a deactivated Cas9 (dCas9) polypeptide.

26. The method of claim 25, wherein the dCas9 polypeptide is linked to a transcriptional activator.

27. The method of claim 25, wherein the dCas9 polypeptide is linked to a transcriptional repressor.

28. The method of claim 26, further comprising constructing a gain-of-function genetic interaction map.

29. The method of claim 27, further comprising constructing a loss-of-function genetic interaction map.


SEQUENCE LISTING (36 sequences)

SEQ ID NO: 1 - DNA, 278 bp, Artificial sequence
  Synthetic construct
tatggcgcgccccaaggtcgggcaggaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaagccagaaacatgg

SEQ ID NO: 2 - DNA, 315 bp, Artificial sequence
  Synthetic construct
gatccgacgcgccatctctaggcccgcgccggccccctcgcacggacttgtgggagaagctcggctactcccctgccccggttaatttgcatataatatttcctagtaactatagaggcttaatgtgcgataaaagacagataatctgttctttttaatactagctacattttacatgataggcttggatttctataacttcgtatagcatacattatacgaagttataaacagcacaaaaggaaactcaccctaactgtaaagtaattgtgtgttttgagactataagtatcccttggagaaccaccttgttgg

SEQ ID NO: 3 - DNA, 291 bp, Artificial sequence
  Synthetic construct
cgtgaccgagcttgtctgcccgcgcagtccactagacagacgcgcagaggtcgggcgggcctagggccaatttcccatggttcccttcatttgcatatatgatgtaataaggttatggagactattaaacttagccctaatcaaactatatgatgataatgtgtgtggtacaaaaggtcataacttattatatactttgaaacttaaaaaagggttacagtttagtcaccataactgtaaaattttttctattcttagctttatatagttcttgagaggccatgtttatgg

SEQ ID NO: 4 - DNA, 318 bp, Artificial sequence
  Synthetic construct
agcgcgcagctcgctgctcccagcatgctccacgggccgagcactcccgcaggaggacccggggccctggggccctcgacgagacccccccttacccaagatgctccgggcgctcatttgcatgtcccaccaagaggtgaactagacttggtttagttgtcgagcaccaaacgtgcgagctttgtgagtgactgcaccaaatcaaatttcccaaaatgttccgcgttcgctggggagaggcgcagcttggtcaccataactataaaggaggaaagatatctgtaccttatatattctgtggggggtccatgattatgg

SEQ ID NO: 5 - DNA, 291 bp, Artificial sequence
  Synthetic construct
ccacgtgaccgagcttgtctgcccgcgcagtccactagacgcacagaggtcggggcgggcctaggaccaatttcccatggttcccttcatttgcatatatgatgtaataaggttatgaagactattaaacttaataataatcaaacaatatgatgatagtgtgtgtggtacaaaagatcataacttattatatactttgaaacttaaagaagggttacagcttagtcaccataactgtaaaattttttctgttcttagctttatatagttcttgagaagccatgtttatgg

SEQ ID NO: 6 - DNA, 297 bp, Artificial sequence
  Synthetic construct
taggggaacgagcaccacgtgaccgagcgtgacagtaaggcgcgcggaggtcgggcgggcagagggccaatttcccatggtcccttcatttgcatataatatgtaggaaggttaaagagacaattctacttaactctcccaaaatcacaaagatatgatgacagtatgtgtgacatagaaggtcataaatcctcgaatacttttaaattgaaaaaaattactcttctatagtcaccataatcaaaaaagtttttatgttcttggctttatataccctctgagaagccacagccgtgg

SEQ ID NO: 7 - DNA, 304 bp, Artificial sequence
  Synthetic construct
tcccggcatgctccgcggggctcagggccccaaaggctccatcgtccgacttggagtcggtgacagggcccttacccaagatgctacgggcactcatttgcatgtcccagcaagaggagaactcgacaagtttcgcgatctagcagtcagaataccaaacatgcgagccttgtgcgtggatctgtgagatcaaatttccctaaaagtttcagagtcatcgaaaagatgcacggtttggtcaccgtaactgtaaagagataagaccttgtgggttttatataagcggtggacgccagctgaatgg

SEQ ID NO: 8 - DNA, 291 bp, Artificial sequence
  Synthetic construct
accacgtgaccgagcttgtctgccctcgcagactactaggcgcgcagaggtcgggcgggggcagggccaatttcccatggttcccttcatttgcatatatgatgtaataaggttatggagactattaaacttaatcataatccaacaatatgatgacagtatgtgtggtacaaaagggcagaacttattatgtactttgagtcttaaagaaggattacagtctagtcaccataagtgtaaaattttttctattcatagctttatatagttcctgagaggccacgtttgtgg

SEQ ID NO: 9 - DNA, 93 bp, Artificial sequence
  Synthetic construct
gtttaagagctaagctggaaacagcatagcaagtttaaataaggctagtccgttatcaacttgaaaaagtggcaccgagtcggtgcttttttt

SEQ ID NO: 10 - DNA, 93 bp, Artificial sequence
  Synthetic construct
gtctaagagctaagctggaaacagcatagcaagtttagataaggctagtccgttatcaacttgaaaaagtggcaccgagtcggtgcttttttt

SEQ ID NO: 11 - DNA, 93 bp, Artificial sequence
  Synthetic construct
gtttcagagctaagctggaaacagcatagcaagttgaaataaggctagtccgttatcaacttgaaaaagtggcaccgagtcggtgcttttttt

SEQ ID NO: 12 - DNA, 93 bp, Artificial sequence
  Synthetic construct
gtttgagagctaagctggaaacagcatagcaagttcaaataaggctagtccgttatcaacttgaaaaagtggcaccgagtcggtgcttttttt

SEQ ID NO: 13 - DNA, 93 bp, Artificial sequence
  Synthetic construct
gtttaagagctaagcacaagagtgcatagcaagtttaaataaggctagtccgttatcaacttgaaaaagtggcaccgagtcggtgcttttttt

SEQ ID NO: 14 - DNA, 93 bp, Artificial sequence
  Synthetic construct
gtttaagagctaagcagaaagctgcatagcaagtttaaataaggctagtccgttatcaacttgaaaaagtggcaccgagtcggtgcttttttt

SEQ ID NO: 15 - DNA, 93 bp, Artificial sequence
  Synthetic construct
gtttaagagctaagcgcgaaagcgcatagcaagtttaaataaggctagtccgttatcaacttgaaaaagtggcaccgagtcggtgcttttttt

SEQ ID NO: 16 - DNA, 94 bp, Artificial sequence
  Synthetic construct
gtttaagagctaagsctggaaacagcatagcaagtttaaataagactagttcgttatcaacttgaaaaagtggcaccgagtcggtgcttttttt

SEQ ID NO: 17 - DNA, 93 bp, Artificial sequence
  Synthetic construct
gtttaagagctaagctggaaacagcatagcaagtttaaataaggctagtccgaattcaacttgaaaaagtggcaccgagtcggtgcttttttt

SEQ ID NO: 18 - DNA, 93 bp, Artificial sequence
  Synthetic construct
gtttaagagctaagctggaaacagcatagcaagtttaaataaggctagtccgtacacaacttgaaaaagtggcaccgagtcggtgcttttttt

SEQ ID NO: 19 - DNA, 93 bp, Artificial sequence
  Synthetic construct
gtttaagagctaagctggaaacagcatagcaagtttaaataaggctagtccgtttacaacttgaaaaagtggcaccgagtcggtgcttttttt

SEQ ID NO: 20 - DNA, 93 bp, Artificial sequence
  Synthetic construct
gtttaagagctaagctggaaacagcatagcaagtttaaataaggctagtccgttatcaacttgagaaagtggcaccgagtcggtgcttttttt

SEQ ID NO: 21 - DNA, 94 bp, Artificial sequence
  Synthetic construct
gtttaagagctaagctggaaacagcatagcaagtttaaataaggctagtccgttatcaacttgaaaaagtgcgcaccgagtcggtgcttttttt

SEQ ID NO: 22 - DNA, 94 bp, Artificial sequence
  Synthetic construct
gtttaagagctaagctggaaacagcatagcaagtttaaataaggctagtccgttatcaacttgaaaaagtgagcaccgagtcggtgcttttttt

SEQ ID NO: 23 - DNA, 95 bp, Artificial sequence
  Synthetic construct
gtttaagagctaagctggaaacagcatagcaagtttaaataaggctagtccgttatcaacttgaaaaagtggcacccgagtcgggtgcttttttt

SEQ ID NO: 24 - DNA, 95 bp, Artificial sequence
  Synthetic construct
gtttaagagctaagctggaaacagcatagcaagtttaaataaggctagtccgttatcaacttgaaaaagtggcagccgagtcggctgcttttttt

SEQ ID NO: 25 - DNA, 95 bp, Artificial sequence
  Synthetic construct
gtttgagagctaagcagaaagctgcatagcaagttcaaataaggctagtccgtacacaacttgaaaaagtggcagccgagtcggctgcttttttt

SEQ ID NO: 26 - DNA, 95 bp, Artificial sequence
  Synthetic construct
gtttcagagctaagcacaagagtgcatagcaagttgaaataaggctagtccgtttacaacttgaaaaagtggcacccgagtcgggtgcttttttt

SEQ ID NO: 27 - DNA, 6 bp, Artificial sequence
  Synthetic construct
aataaa

SEQ ID NO: 28 - DNA, 8 bp, Artificial sequence
  Synthetic construct
tgacgtgg

SEQ ID NO: 29 - DNA, 20 bp, Artificial sequence
  Synthetic construct
gaccaggatgggcaccaccc

SEQ ID NO: 30 - DNA, 20 bp, Artificial sequence
  Synthetic construct
ggggatctgagaatgtacca

SEQ ID NO: 31 - DNA, 20 bp, Artificial sequence
  Synthetic construct
gcgggctgagacgtggccag

SEQ ID NO: 32 - DNA, 20 bp, Artificial sequence
  SEQ ID NO 33
gagaactgactaggcagcgg

SEQ ID NO: 33 - DNA, 20 bp, Artificial sequence
  SEQ ID NO 34
gacgactagttaggcgtgta

SEQ ID NO: 34 - DNA, 20 bp, Artificial sequence
  Synthetic construct
ggccaaacgtgccctgacgg

SEQ ID NO: 35 - DNA, 20 bp, Artificial sequence
  Synthetic construct
gccttggctaaaccgctccc

SEQ ID NO: 36 - DNA, 20 bp, Artificial sequence
  Synthetic construct
aactttcagtttagcggtct